SEQUENCE LISTING (4 sequences)

SEQ ID NO: 1 - DNA, 22 bp, Human immunodeficiency virus type 1
bgtgtggaaaatctctagcagt

SEQ ID NO: 2 - DNA, 21 bp, Human immunodeficiency virus type 1
actgctagagattttccacac

SEQ ID NO: 3 - DNA, 20 bp, Human immunodeficiency virus type 1
tgaccaagggctaattcact

SEQ ID NO: 4 - DNA, 20 bp, Human immunodeficiency virus type 1
agtgaattagcccttggtca

(19) United States
(12) Patent Application Publication
(10) Pub. No.: US 2006/0211724 A1
(43) Pub. Date: Sep. 21, 2006

(54) Hiv integrase inhibitors

(76) Inventors: Gaston Wim Verschueren, Berchem (BE); Inge Dierynck, Antwerpen (BE); Dominique Louis Nestor Ghislain Surleraux, Braine-le-Chateau (BE); Frederik Frans Desire Daeyaert, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/554,712

(22) PCT Filed: Apr. 27, 2004
(86) PCT No.: PCT/EP04/50621

§ 371 Date: Oct. 27, 2005

(30) Foreign Application Priority Data
Apr. 28, 2003 (EP) 03101164.6

(51) Int. Cl. C07D 471/02; C07D 491/02; A61K 31/4745; A61K 31/407

(52) U.S. Cl. 514/291; 514/411; 546/80; 548/430

(57) ABSTRACT

The present invention concerns the compounds having the formula (1), N-oxides, salts, stereoisomeric forms, racemic mixtures, prodrugs, esters and metabolites thereof wherein (a) or (b); A, together with the two carbons of the phenyl ring to which it is attached forms a monocyclic aryl or a monocyclic Het²; R¹ is hydrogen, halo, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl, optionally polysubstituted C₂₋₆alkenyl or optionally polysubstituted C₂₋₆alkynyl; R² is hydrogen, C₃₋₇cycloalkyl, aryl, Het¹, Het², C(═O)—R⁵, S(═)_{y}—R⁶OR⁷, NR⁸R⁹, C═NR⁸)—R⁵, or optionally polysubstituted C₁₋₆alkyl, optionally polysubstituted C₂₋₆alkenyl or optionally polysubstituted C₂₋₆alkynyl. It further relates to their use as HIV integrase inhibitors, processes for their preparation as well as pharmaceutical compositions and diagnostic kits comprising them. It also concerns combinations thereof with other anti-retroviral agents, and to their use in assays as reference compounds or as reagents.

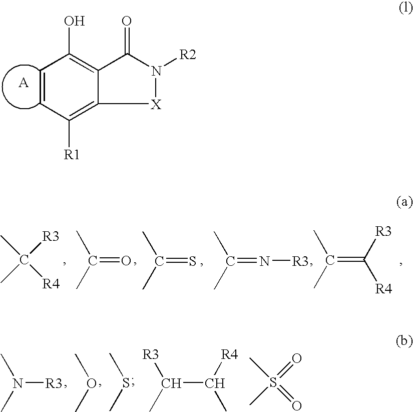

The present invention relates to novel compounds, their use as integrase inhibitors, processes for their preparation as well as pharmaceutical compositions and diagnostic kits comprising them. The present invention also concerns combinations of the present integrase inhibitors with anti-retroviral agents. It further relates to their use in assays as reference compounds or as reagents. The compounds of the present invention are useful for preventing or treating infection by HIV and for treating AIDS.

The virus causing the acquired immunodeficiency syndrome (AIDS) is known by different names, including T-lymphocyte virus III (HTLV-III) or lymphadenopathy-associated virus (LAV) or AIDS-related virus (ARV) or human immunodeficiency virus (HIV). Distinct families have been identified, such as HIV-1 and HIV-2. Hereinafter, HIV will be used to generically denote these viruses.

A common feature of retrovirus replication is the insertion by virally-encoded integrase of proviral DNA into the host cell genome, a required step in HIV replication in human T-lymphoid and monocytoid cells. The integration process takes place following reverse transcription of the viral RNA. First, the viral integrase binds to the viral DNA and removes two nucleotides from the 3′ end of the viral long-terminal repeat (LTR) sequences on each strand. This step is called 3′ end processing and occurs in the cytoplasm within a nucleoprotein complex termed the pre-integration complex (PIC). Second, in a process called strand transfer, the two strands of the cellular DNA into which the viral DNA will be inserted, i.e. the target DNA, are cleaved in a staggered fashion. The 3′ ends of the viral DNA are ligated to the 5′ ends of the cleaved target DNA. Finally, remaining gaps are repaired, probably by cellular enzymes.

It is known that some antiviral compounds which act as inhibitors of HIV replication are effective agents in the treatment of AIDS and similar diseases, including reverse transcriptase inhibitors such as azidothymidine (AZT), ddC, stavudine, didanosine, nevirapine, abacavir, lamivudine, delavirdine, tenofovir and efavirenz and protease inhibitors such as indinavir, saquinavir, anprenavir, lopinavir, ritonavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells.

The compounds of the present invention specifically inhibit HIV integrase and HIV replication and not only are they active against wild-type HIV virus, but they also show activity against various mutant HIV viruses.

Other HIV integrase inhibitors are known in the art. For instance, WO0255079, WO0230931, WO0230930 and WO0230426 (all by Merck & Co., Inc.) disclose aza- and polyaza-naphthalenyl carboxamides useful as inhibitors of HIV integrase. WO0236734 (by Merck & Co., Inc.) discloses additionally aza- and polyaza-naphthalenyl ketones useful as inhibitors of HIV integrase. In Roggo et al., Journal of antibiotics (1996), spirodihydrobenzofuranlactams are disclosed as antagonists of endothelin and as inhibitors of HIV-1 protease.

EP0459449 by Shionogi & Co., discloses furano[2,3-F]isoindoles as aldose reductase inhibitors. CS225002 (by Krepelka Jiri and Vlckova Drahuse) discloses 9-phenyl-1H-benzo[f]isoindole-1,3-dione derivatives capable of inhibiting tumors in mice and rats. Similarly, CS210880 (by Krepelka Jiri, Vancurova Iva and Roubik Jiri) discloses certain 4-arylnaphthalene-2,3-dicarboxylic acid imides as antineoplastic active compounds. The article by Krepelka et al., Collect. Czech. Chem. Commun. (1982), 47(1), pp 304-14 discloses the synthesis and neoplastic effects of some N-substituted imides of 1-substituted 4-arylnaphthalene-2,3-dicarboxylic acids.

Hartmann et al. describe the preparation of naphthoquinone imines as NIR dyes, in Tetrahedron, Vol. 51, No. 16. Kappe et al. disclose the generation and subsequent cycloaddition chemistry of alpha-amino isobenzofurans formed by cationic cyclization, in Tetrahedron Letters, Vol. 36, No. 51. Padwa et al. have published studies dealing with the cycloaddition/ring opening/elimination sequence of 2-amino-substituted isobenzofuranes, in J. Org. Chem., Vol. 62. Passannanti et al. describe the synthesis of narciclastic aldehyde and related isocarbostyrils in J. Heterocyclic Chemistry, Vol. 14, No. 1.

The present invention concerns novel compounds having the formula (I), 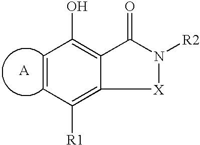
and their N-oxides, salts, stereoisomeric forms, racemic mixtures, prodrugs, esters and metabolites thereof, wherein
X is 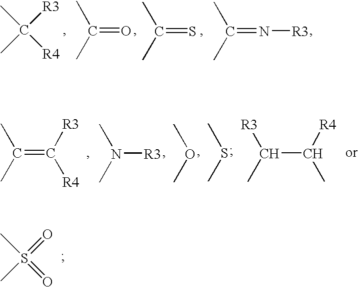
A, also mentioned as “A-ring”, together with the two carbons of the phenyl ring to which it is attached forms a monocyclic aryl or a monocyclic Het²;
R¹ is hydrogen, halogen, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R² is hydrogen, C₃₋₇cycloalkyl, aryl, Het¹, Het², C(═O)—R⁵, S(—O)_{y}—R⁶, OR⁷, NR⁸R⁹, C(═NR⁸)—R⁵, or optionally polysubstituted C₁₋₆alkyl, optionally polysubstituted C₂₋₆alkenyl or optionally polysubstituted C₂₋₆alynyl; whereby the optional substituents on C₁₋₆alkenyl, C₂₋₆alkenyl and C₂₋₆alkynyl are each independently selected from halogen, nitro, cyano, C₃₋₇cycloalkyl, aryl, Het¹, Het², C(═O)—R⁵, S(—O)_{y}—R⁶, OR⁷, and NR⁸R⁹;
R³ is hydrogen, halogen, nitro, cyano, C₃₋₇cycloalkyl, aryl, C(═O)—R⁵, S(═O)_{y}—R⁶, OR⁷, NR⁸R⁹, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, C(═O)—R⁵, OR⁷, and NR⁸R⁹;
R⁴ is hydrogen, halogen, nitro, cyano, C₃₋₇cycloalkyl or C₁₋₆alkyl;
y represents an integer being zero, one or two;
R⁵ is hydrogen, C₃₋₇cycloalkyl, aryl, Het¹, Het², C(═O)—R¹⁰, OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂-alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³; R⁶ is hydrogen, aryl, C₃₋₇cycloalkyl, Het¹, Het², OR¹², NR⁸R¹³, optionally polysubstituted C₁₋₄alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁷ is hydrogen, aryl C₃₋₇cycloalkyl, Het¹, Het², C(═O)—R¹⁰, S(═O)_{y}—R¹¹, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁸ is hydrogen, aryl, Het¹, Het², C₁₋₆alkyl C₂₋₆alkenyl, C₂₋₆alkynyl, C₃₋₇cycloalkyl or polyhaloC₁₋₆alkyl;
R⁹ is hydrogen, aryl, C₃₋₇cycloalkyl, Het¹, Het², C(═O)—R¹⁰, S(═O)_{y}—R¹¹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(—O)—R¹⁰, S(═O)_{y}—R¹¹, OR¹² and NR⁸R¹³;
R¹⁰ is hydrogen, C₃₋₇cycloalkyl, aryl, Het¹, Het², C(═O)—R⁸, C(—O)—OR⁸, C(═O)—NR⁸R⁸; OR⁸, O—C(═O)—R⁸, O—S(═O)_{y}—R⁸, S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)—R⁸, NR⁸R⁸, NR⁸—C(═O)—R⁸, and NR⁸—S(═O)_{y}—R⁸;
R¹¹ is hydrogen, C₃₋₇cycloalkyl, aryl, Het¹, Het², OR⁸, O—C(═O)—R⁸, O—S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O), —R⁸, S(═O)_{y}—OR⁸, S(═O)_{y}—NR³R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹² is hydrogen, C₃₋₇cycloalkyl, aryl, Het¹, Het², C(═O)—R⁸, C(═O)—OR⁸, C(═O)—NR⁸R⁸; S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(—O)_{y}—NR⁸R⁸, OR⁸, O—C(═O)—R⁸, O—S(—O)_{y}—R⁸, NR⁸R⁸, NR⁸—C(═O)—R⁸, and NR⁸—S(═O)_{y}—R⁸;
R¹³ is hydrogen, C₃₋₇cycloalkyl, aryl, Het¹, Het², C(═O)—R⁸, C(═O)_OR⁸, C(═O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂-alkenyl or optionally polysubstituted C₂-alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(—O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(=)—R⁸, O—S(═O)_{y}—R⁸, NR⁸R⁸, NR⁸—C(═O)—R⁸, and NR⁸—S(═O)_{y}—R⁸;
R¹⁴ is hydrogen, phenyl, C₁₋₆alkyl, C₂₋₆alkenyl, C₂₋₆alkynyl, C₃₋₇cycloalkyl;
aryl as a group or part of a group represents a monocyclic or polycyclic aromatic or a partially saturated monocyclic or polycyclic carbocycles wherein any such carbocycle within the meaning of aryl may have up to 14 carbon atoms and may be optionally substituted with one or more substituents independently selected from halogen, nitro, oxo, cyano, C₃₋₇cycloalkyl, Het¹, Het², C(═O)—R⁸, S(═O)_{y}—R¹⁴, OR¹⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het², and NR¹⁴R¹⁴; and whereby the optional substituents on phenyl are each independently selected from halogen, hydroxy, C₁₋₆alkyl, polyhaloC₁-alkyl, O—C₁₋₆alkyl, and C₁₋₆alkanediyl-NR¹⁴R¹⁴;
Het¹ as a group or part of a group represents a saturated or partially unsaturated monocyclic, bicyclic or tricyclic heterocycle having 3 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₄alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het² as a group or part of a group represents an aromatic monocyclic, bicyclic or tricyclic heterocycle having 5 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴ OR¹⁴, NR¹⁴R¹⁴, NR¹⁴—O—C(═O)—R¹⁴, optionally polysubstituted C₁-alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
   with the proviso that compounds:
9-(2,4-Dimethoxy-phenylimino)-9H-benzo[f]isoindole-1,3,4-trione,
9-(2,4-Dimethoxy-phenylimino)-2-phenyl-9H-benzo[f]isoindole-1,3,4-trione,
6,7-Dichloro-9-(2,4-dimethoxy-phenylimino)-2-phenyl-9H-benzo[f]isoindole-1,3,4-trione,
4-[6,7-Dichloro-4-(2,4-dimethoxy-phenylimino)-1,3,9-trioxo-1,3,4,9-tetrahydro-benzo[f]isoindol-2-yl]-benzonitrile,
6,7-Dichloro-9-(4-methoxy-2-methyl-phenylimino)-2-phenyl-9H-benzo[f]isoindole-1,3,4-trione,
9-(4-Dimethylamino-phenyl imino)-2-phenyl-9H-benzo[f]isoindole-1,3,4-trione,
4-Diethylamino-9-hydroxy-2-phenyl-benzo[f]isoindole-1,3-dione,
4-(But-3-enyl-ethyl-amino)-9-hydroxy-2-phenyl-benzo[f]isoindole-1,3-dione,
4-(Ethyl-pent-4-enyl-amino)-9-hydroxy-2-phenyl-benzo[f]isoindole-1,3-dione,
4,9-dihydroxy-2-methyl-benzo[f]isoindole-1,3-dione,
4,8-dihydroxy-6-methyl-2-oxa-6-aza-s-indacene-5,7-dione,
5,9-dihydroxy-7-methyl-pyrrolo[3,4-g]quinoline-6,8-dione,
4,9-dihydroxy-2-methyl-pyrrolo[3,4-g]isoquinoline-1,3-dione,
4,9-dihydroxy-2,6-dimethyl-benzo[f]isoindole-1,3-dione,
4,9-dihydroxy-6-methoxy-2-methyl-benzo[f]isoindole-1,3-dione,
5-fluoro-4,9-dihydroxy-2-methyl-benzo[f]isoindole-1,3-dione,
6,7-dichloro-4,9-dihydroxy-2-methyl-benzo[f]isoindole-1,3-dione,
6-cyclohexyl-4,8-dihydroxy-1-thia-6-aza-s-indacene-5,7-dione,
4,9-dihydroxy-6-methyl-2-phenyl-benzo[f]isoindole-1,3-dione,
7-cyclohexyl-5,9-dihydroxy-pyrrolo[3,4-g]quinoline-6,8-dione,
2-cyclohexyl-4,9-dihydroxy-6-methoxy-benzo[f]isoindole-1,3-dione,
7-(3,5-dichloro-phenyl)-5,9-dihydroxy-pyrrolo[3,4-g]quinoline-6,8-dione,
6,7-dichloro-2-(3,5-dichloro-phenyl)-4,9-dihydroxy-benzo[f]isoindole-1,3-dione,
4-hydroxy-benzo[f]isoindole-1,3-dione,
4-hydroxy-2-phenyl-benzo[f]isoindole-1,3-dione,
4-hydroxy-2-phenyl-9-phenylamino-benzo[f]isoindole-1,3-dione,
4,9-dihydroxy-2-phenyl-benzo[f]isoindole-1,3-dione,
4-hydroxy-1-methyl-2-phenyl-1,2-dihydro-benzo[f]indazol-3-one,
6,7-dichloro-4,9-dimethoxy-2-methyl-benzo[f]isoindole-1,3-dione, and
6,7-dichloro-2-(3,5-dichloro-phenyl)-4,9-dimethoxy-benzo[f]isoindole-1,3-dione, are excluded.

The present invention also concerns novel compounds having the formula (I), 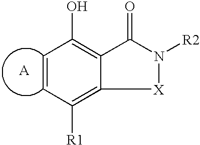
and their N-oxides, salts, stereoisomeric forms, racemic mixtures, prodrugs, esters and metabolites thereof, wherein
X, A, R¹, R², R³, R⁴, R⁵, R⁶, R⁷, R⁸, R⁹, R¹⁰, R¹¹, R¹², R¹³, R¹⁴, y, aryl, Het¹, and Het² are as defined above, provided that when the A-ring is phenyl, then R² is not hydrogen, methyl, cyclohexyl, nor phenyl; and that compounds
4,8-dihydroxy-6-methyl-2-oxa-6-aza-s-indacene-5,7-dione,
5,9-dihydroxy-7-methyl-pyrrolo[3,4-g]quinoline-6,8-dione,
4,9-dihydroxy-2-methyl-pyrrolo[3,4-g]isoquinoline-1,3-dione,
6-cyclohexyl-4,8-dihydroxy-1-thia-6-aza-s-indacene-5,7-dione,
7-cyclohexyl-5,9-dihydroxy-pyrrolo[3,4-g]quinoline-6,8-dione,
7-(3,5-dichloro-phenyl)-5,9-dihydroxy-pyrrolo[3,4-g]quinoline-6,8-dione, are excluded.

The present invention also concerns novel compounds having the formula (I), 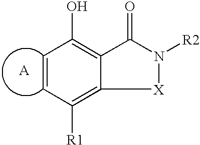
and their N-oxides, salts, stereoisomeric forms, racemic mixtures, prodrugs, esters and metabolites thereof, wherein
X is 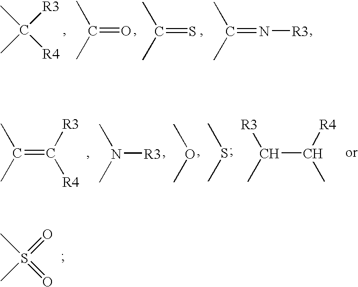
A, also mentioned as “A-ring”, together with the two carbons of the phenyl ring to which it is attached forms a monocyclic Het²;
R¹ is hydrogen, halogen, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R² is hydrogen, C₃₋₅cycloalkyl, C₃₋₇cycloalkyl, aryl, Het¹, Het², C(═O)—R⁵, S(═O)_{y}—R⁶, OR⁷, NR⁸R⁹, C(═NR⁵)—R⁵, C₂₋₆alkyl or polysubstituted C₁₋₆alkyl, optionally polysubstituted C₂₋₆alkenyl or optionally polysubstituted C₂₋₆alkynyl; whereby the substituents on C₁₋₆alkyl, and the optional substituents on C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R³ is hydrogen, halogen, nitro, cyano, C₃₋₇cycloalkyl, aryl, C(═O)—R⁵, S(═O)_{y}—R⁶, OR⁷, NR⁸R⁹, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, C(═O)—R⁵, OR⁷, and NR⁸R⁹;
R⁴ is hydrogen, halogen, nitro, cyano, C₃₋₇cycloalkyl or C₁₋₆alkyl;
y represents an integer being zero, one or two;
R⁵ is hydrogen, C₃₋₇cycloalkyl, aryl, Het¹, Het², C(═O)—R¹⁰, OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁶ is hydrogen, aryl, C₃₋₇cycloalkyl, Het¹, Het², OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁷ is hydrogen, aryl, C₃₋₇cycloalkyl, Het¹, Het², C(═O)—R¹⁰, S(═O)_{y}—R¹¹, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁸ is hydrogen, aryl, Het¹, Het², C₁₋₆alkyl, C₂₋₆alkenyl, C₂₋₆alkynyl, C₃₋₇cycloalkyl or polyhaloC₁₋₆alkyl;
R⁹ is hydrogen, aryl, C₃₋₇cycloalkyl, Het¹, Het², C(═O)—R¹⁰, S(═O)_{y}—R¹¹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹² and NR⁸R¹³;
R¹⁰ is hydrogen, C₃₋₇cycloalkyl, aryl, Het¹, Het², C(═O)—R⁸, C(═O)—OR⁸, C(—O)—NR³R⁸, OR⁸, O—C(═O)—R⁸, O—S(═O), —R⁸, S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(—O)_{y}—R⁸, NR⁸R⁸, NR⁸—C(═O)—R⁸, and NR⁸—S(═O)_{y}—R⁸;
R¹¹ is hydrogen, C₃₋₇cycloalkyl, aryl, Het¹, Het², OR⁸, O—C(═O)—R⁸, O—S(═O)_{y}—R⁸, NR⁸R⁸, NR⁸—C(═O)—R⁸, NR⁸—S(—O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹² is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(—O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O), —OR⁸, S(═O), —NR⁸R⁸, OR⁸, O—C(═O)—R⁸, O—S(═O)_{y}—R⁸, NR⁸R⁸, NR⁸—C(═O)—R⁸, and NR⁸—S(═O)Y—R⁸;
R¹³ is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁹, O—S(═O)_{y}—R⁸, NR⁸R⁸, NR⁸—C(═O)—R⁸, and NR⁸—S(═O)_{y}—R⁸;
R¹⁴ is hydrogen, phenyl, C₁₋₆alkyl, C₂₋₆alkenyl, C₂₋₆alkynyl, C₃₋₇cycloalkyl;
aryl as a group or part of a group represents a monocyclic or polycyclic aromatic or a partially saturated monocyclic or polycyclic carbocycles wherein any such carbocycle within the meaning of aryl may have up to 14 carbon atoms and may be optionally substituted with one or more substituents independently selected from halogen, nitro, oxo, cyano, C₃₋₇cycloalkyl, Het¹, Het², C(═O)—R⁸, S(═O)_{y}—R¹⁴, OR¹⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het¹ as a group or part of a group represents a saturated or partially unsaturated monocyclic, bicyclic or tricyclic heterocycle having 3 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het² as a group or part of a group represents an aromatic monocyclic, bicyclic or tricyclic heterocycle having 5 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
   with the proviso that compound 7-(3,5-dichloro-phenyl)-5,9-dihydroxy-pyrrolo-[3,4-g]quinoline-6,8-dione is excluded.

In one embodiment, the present invention concerns compounds for use in therapy, in particular for the manufacture of a medicament for treating or combating infection or disease associated with retrovirus infection in a mammal, having the formula (I), 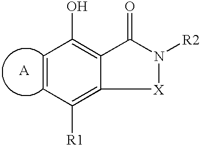
and their N-oxides, salts, stereoisomeric forms, racemic mixtures, prodrugs, esters and metabolites thereof, wherein
X is 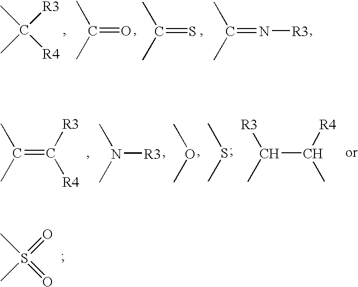
A, also mentioned as “A-ring”, together with the two carbons of the phenyl ring to which it is attached forms a monocyclic aryl or a monocyclic Het²;
R¹ is hydrogen, halogen, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl; optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R² is hydrogen, C₃₋₇cycloalkyl, aryl, Het¹, Het², C(═O)—R⁵, S(═O)_{y}—R⁶, OR⁷, NR⁸R⁹, C(═NR⁸)—R⁵, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹; R³ is hydrogen, halogen, nitro, cyano, C₃₋₇cycloalkyl, aryl, C(═O)—R⁵, S(═O)_{y}—R⁶, OR⁷, NR⁸R⁹, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, C(═O)—R⁵, OR⁷, and NR⁸R⁹; R⁴ is hydrogen, halogen, nitro, cyano, C₃₋₇cycloalkyl or C₁₋₆alkyl;
y represents an integer being zero, one or two;
R⁵ is hydrogen, C₃₋₇cycloalkyl, aryl, Het¹, Het², C(═O)—R¹⁰, OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁶ is hydrogen, aryl, C₃₋₇cycloalkyl, Het¹, Het², OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁷ is hydrogen, aryl, C₃₋₇cycloalkyl, Het¹, Het², C(═O)—R¹⁰, S(═O)_{y}—R¹¹, or optionally polysubstituted C₁₋₆ alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(—O)—R¹⁰, S(═O)_{y}—R¹¹, OR¹², and NR⁸R¹³; R⁸ is hydrogen, aryl, Het¹, Het², C₁₋₆alkyl, C₂₋₆alkenyl, C₂₋₆alkynyl, C₃₋₇cycloalkyl or polyhaloC₁₋₆alkyl;
R⁹ is hydrogen, aryl, C₃₋₇cycloalkyl, Het¹, Het², C(═O)—R¹⁰, S(═O)_{y}—R¹¹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(—O)—R¹⁰, S(═O)_{y}—R¹¹, OR¹² and NR⁸R¹³;
R¹⁰ is hydrogen, C₃₋₇cycloalkyl, aryl, Het¹, Het², C(═O)—R⁸, C(═O)—OR⁸, C(═O)—NR⁸R⁸, OR⁸, O—C(═O)—R⁸, O—S(═O)_{y}—R⁸, S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₄alkenyl and C₂₋₆alkynyl are each independently selected from halogen, nitro, cyano, C₃₋₇cycloalkyl, aryl, Het¹, Het², C(═O)—R⁸, C(O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(—O)_{y}—R⁸, NR⁸R⁸, NR⁸—C(═O)—R⁸, and NR⁸—S(═O)_{y}—R⁸;
R¹¹ is hydrogen, C₃₋₇cycloalkyl, aryl, Het¹, Het², OR⁸, O—C(═O)—R⁸, O—S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(—O)—NR⁸R⁸, S(═O)_{y}—R⁸, S(═O)_{y}—OR⁸, S(═O)_{y}—NR⁸R⁸, OR⁸, O—C(═O)—R⁸, O—S(—O)_{y}—R⁸, NR⁸R⁸, NR⁸—C(═O)—R⁸, and NR⁸—S(═O)_{y}—R⁸;
R¹² is hydrogen, C₃₋₇cycloalkyl, aryl, Het¹, Het², C(=)—R⁸, C(═O)—OR⁸, C(—O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(—O)—NR⁸R⁸, S(═O)_{y}—R⁸, S(═O)_{y}—OR⁸, S(═O)_{y}—NR⁸R⁸, OR⁸, O—C(—O)—R⁸, O—S(═O)_{y}—R⁸, NR⁸R⁸, NR⁸—C(═O)—R⁸, and NR⁸—S(═O)_{y}—R⁸, S(═O)_{y}—R⁸;
R¹³ is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O), —NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(—O)_{y}—R⁸, NR⁸R⁸, NR⁸—C(═O)—R⁸, and NR⁸—S(═O)_{y}—R⁸;
R¹⁴ is hydrogen, phenyl, C₁₋₆alkyl, C₂₋₆alkenyl, C₂₋₆alkynyl, C₃₋₇cycloalkyl;
aryl as a group or part of a group represents a monocyclic or polycyclic aromatic or a partially saturated monocyclic or polycyclic carbocycles wherein any such carbocycle within the meaning of aryl may have up to 14 carbon atoms and may be optionally substituted with one or more substituents independently selected from halogen, nitro, oxo, cyano, C₃₋₇cycloalkyl, Het¹, Het², C(═O)—R⁸, S(═O)_{y}—R¹⁴, OR¹⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴; Het¹ as a group or part of a group represents a saturated or partially unsaturated monocyclic, bicyclic or tricyclic heterocycle having 3 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(—O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het² as a group or part of a group represents an aromatic monocyclic, bicyclic or tricyclic heterocycle having 5 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(=)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₄alkanediyl-NR¹⁴R¹⁴.

In yet another embodiment, the present invention concerns pharmaceutical formulations comprising the compounds having the formula (I), 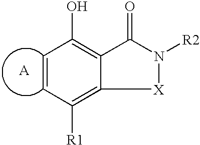
and their N-oxides, salts, stereoisomeric forms, racemic mixtures, prodrugs, esters and metabolites thereof, wherein
X, A, R¹, R², R³, R⁴, y, R⁵, R⁶, R⁷, R⁸, R⁹, R¹⁰, R¹¹, R¹², R¹³, R¹⁴, aryl, Het¹, and Het² are as defined above.

This invention also concerns the quaternization of the nitrogen atoms of the present compounds. A basic nitrogen can be quaternized with any agent known to those of ordinary skill in the art including, for instance, lower alkyl halides, dialkyl sulfates, long chain halides and arylalkyl halides.

As used herein, the term “halo” or “halogen” as a group or part of a group is generic for fluoro, chloro, bromo or iodo.

The term sultam defines a cyclic aminosulfonyl group. Examples of a sultam are 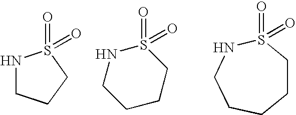
and they may be attached to the remainder of the molecule via the nitrogen atom or a carbon atom.

The term sultim defines a cyclic aminosulfoxyl group. Examples of a sultim are 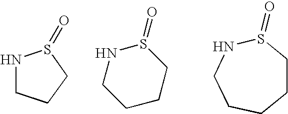
and they may be attached to the remainder of the molecule via the nitrogen atom or a carbon atom.

The term “C₁₋₂alkyl” is generic to methyl or ethyl.

The term “C₁₋₃alkyl” as a group or part of a group defines saturated hydrocarbon radicals having from 1 to 3 carbon atoms, such as the groups defined for C₁₋₂alkyl, propyl, isopropyl, and the like.

The term “C₁₋₄alkyl” as a group or part of a group defines straight and branched chained saturated hydrocarbon radicals having from 1 to 4 carbon atoms, such as the groups defined for C₁₋₃alkyl, butyl, 2-methyl-propyl, and the like.

The term “C₂₋₄alkyl” as a group or part of a group defines straight and branched chained saturated hydrocarbon radicals having from 2 to 4 carbon atoms, such as, for example, ethyl, propyl, butyl, 2-methyl-propyl, and the like.

The term “C₁₋₆alkyl” as a group or part of a group defines straight and branched chained saturated hydrocarbon radicals having from 1 to 6 carbon atoms. Examples of C₁₋₆alkyl are the groups defined for C₁₋₄alkyl, pentyl, hexyl, 2-methylbutyl, 3-methylpentyl, and the like.

The term “C₂₋₆alkyl” as a group or part of a group defines straight and branched chained saturated hydrocarbon radicals having from 2 to 6 carbon atoms, such as the groups defined for C₂₋₄alkyl, pentyl, hexyl, 2-methylbutyl, 3-methylpentyl, and the like.

The term “C₃₋₆alkyl” as a group or part of a group defines straight and branched chained saturated hydrocarbon radicals having from 3 to 6 carbon atoms, such as propyl, pentyl, hexyl, 2-methylbutyl, 3-methylpentyl, and the like.

The term “C₁₋₂alkanediyl” is generic to methanediyl, 1,2-ethanediyl, or 1,1-ethanediyl.

The term “C₁₋₃alkanediyl” as a group or part of a group defines bivalent hydrocarbons having from 1 to 3 carbon atoms, such as the groups defined for C₁₋₂alkanediyl, 1,3-propanediyl, and the like.

The term “C₁₋₄alkanediyl” as a group or part of a group defines bivalent straight and branched chained hydrocarbons having from 1 to 4 carbon atoms, such as the groups defined for C₁₋₃alkanediyl, 1,3-butanediyl, 1,4-butanediyl, and the like.

The term “C₁₋₆alkanediyl” as a group or part of a group defines bivalent straight and branched chained hydrocarbons having from 1 to 6 carbon atoms, such as the groups defined for C₁₋₄alkanediyl, 1,3-pentanediyl, 1,5-pentanediyl, 1,4-hexanediyl, 1,6-hexanediyl, and the like.

The term “C₂₋₄alkaneyl” as a group or part of a group defines bivalent straight and branched chained hydrocarbons having from 2 to 4 carbon atoms such as, for example, 1,2-ethanediyl, 1,3-propanediyl, 1,3-butanenediyl, 1,4-butanediyl, and the like.

The term “C₂₋₆alkanediyl” as a group or part of a group defines bivalent straight and branched chained hydrocarbons having from 2 to 6 carbon atoms, such as the groups defined for C₂₋₄alkanediyl, 1,3-pentanediyl, 1,5-pentanediyl, 1,4-hexanediyl, 1,6-hexanediyl, and the like.

The term “C₂₋₃alkenyl” as a group or part of a group defines hydrocarbon radicals having 2 or 3 carbon atoms containing at least one double bond such as, for example, ethenyl, propenyl, and the like.

The term “C₂₋₅alkenyl” as a group or part of a group defines hydrocarbon radicals having from 2 to 5 carbon atoms containing at least one double bond such as the groups defined for C₂₋₃alkenyl, butenyl, pentenyl and the like.

The term “C₂₋₆alkenyl” as a group or part of a group defines straight and branched chained hydrocarbon radicals having from 2 to 6 carbon atoms containing at least one double bond such as the groups defined for C₂₋₅alkenyl, hexenyl and the like.

The term “C₂₋₅alkenediyl” as a group or part of a group defines bivalent straight and branched chained hydrocarbons having from 2 to 5 carbon atoms containing at least one double bond such as, for example, 1,2-ethenediyl, 1,3-propenediyl, 1,3-butenediyl, 1,4-butenediyl, 1,2-pentenediyl, 1,5-pentenediyl and the like.

The term “C₂₋₆alkenediyl” as a group or part of a group defines bivalent straight and branched chained hydrocarbons having from 2 to 6 carbon atoms containing at least one double bond such as the groups defined for C₂₋₅alkenediyl, 1,4-hexenediyl, 1,6-hexenediyl, and the like.

The term “C₂₋₃alkynyl” as a group or part of a group defines hydrocarbon radicals having 2 or 3 carbon atoms containing at least one triple bond such as, for example, ethynyl, propynyl and the like.

The term “C₂₋₅alkynyl” as a group or part of a group defines straight and branched chained hydrocarbon radicals having from 2 to 5 carbon atoms containing at least one triple bond such as the groups defined for C₂₋₃alkynyl, butynyl, pentynyl and the like.

The term “C₂₋₆alkynyl” as a group or part of a group defines straight and branched chained hydrocarbon radicals having from 2 to 6 carbon atoms containing at least one triple bond such as the groups defined for C₂₋₅alkynyl, hexynyl and the like.

The term “C₂₋₅alkynydiyl” as a group or part of a group defines bivalent straight and branched chained hydrocarbons having from 2 to 5 carbon atoms containing at least one triple bond such as, for example, 1,2-ethynydiyl, 1,3-propynydiyl, 1,3-butynydiyl, 1,4-butynydiyl, 1,4-pentynydiyl, 1,5-pentynydiyl and the like.

The term “polyhaloC₁₋₄alkyl” as a group or part of a group, defines a C₁₋₄alkyl radical having the meaning as defined above wherein one or more hydrogen atoms are replaced with a halogen, preferably a bromo, chloro or fluoro atom. The term “polyhaloC₁₋₄alkyl” is also equivalent to the expression “C₁₋₄alkyl optionally substituted with one or more substituents independently selected from halogen”. Examples of such polyhaloC₁₋₄alkyl radicals include chloromethyl, 1-bromoethyl, fluoromethyl, difluoromethyl, trifluoromethyl, 1,1,1-trifluoroethyl and the like.

The term “polyfluoroC₁₋₄alkyl” as a group or part of a group, defines a C₁₋₄alkyl radical having the meaning as defined above wherein one or more hydrogen atoms are replaced with a fluoro atom.

The term “polyhaloC₁₋₆alkyl” as a group or part of a group, defines a C₁₋₆alkyl radical having the meaning as defined above wherein one or more hydrogen atoms are replaced with a halogen, preferably a bromo, chloro or fluoro atom. The term “polyhaloC₁₋₆alkyl” is also equivalent to the expression “C₁₋₆alkyl optionally substituted with one or more substituents independently selected from halogen”. Examples of such polyhaloC₁₋₆alkyl radicals include the groups defined for 3-fluoropentyl, 2-chloro-6-bromohexyl, and the like.

The term “polyfluoroC₁₋₆-alkyl” as a group or part of a group, defines a C₁₋₆alkyl radical having the meaning as defined above wherein one or more hydrogens are replaced with a fluoro atom.

The term “C₃₋₆cycloalkyl” as a group or part of a group is generic to cyclopropyl, cyclobutyl, cyclopentyl, cyclohexyl.

The term “C₃₋₅cycloalkyl” as a group or part of a group is generic to cyclopropyl, cyclobutyl, cyclopentyl.

The term “C₃₋₇cycloalkyl” as a group or part of a group is generic to cyclopropyl, cyclobutyl, cyclopentyl, cyclohexyl, cycloheptyl.

The term “C₇cycloalkyl” as a group or part of a group is generic to cycloheptyl.

Examples of aryl include phenyl and naphtyl, or 1,2,3,4-tetrahydro-naphthalene, 1,2-dihydro-naphthalene, naphthalene, indan, 1H-indene, bicyclo[4.2.0]octa 1,3,5-triene, 6,7,8,9-tetrahydro-5H-benzocycloheptene, 6,7-dihydro-5H-benzocycloheptene.

Whenever the terms “polysubstituted” and “one or more substituents” are used in defining the compounds of the present invention, unless otherwise stated, it is meant to indicate that one or more hydrogens on the atom indicated in the expression using ‘polysubstituted’ and “one or more substituents” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

When any variable (e.g. halogen or C₁₋₆alkyl) occurs more than one time in any constituent, each definition is independent.

The term “prodrug” as used throughout this text means the pharmacologically acceptable derivatives such as esters, amides and phosphates, such that the resulting in vivo biotransformation product of the derivative is the active drug as defined in the compounds of the present invention. The reference by Goodman and Gilman (The Pharmacological Basis of Therapeutics, 8^{th} ed, McGraw-Hill, Int. Ed. 1992, “Biotransformation of Drugs”, pp 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs include compounds of the present invention wherein a hydroxy group, or an amino group is bonded to any group that, when the prodrug is administered to a patient, cleaves to form a free hydroxyl or free amino, respectively.

Prodrugs are characterized by excellent aqueous solubility, increased bioavailability and are readily metabolized into the active inhibitors in vivo.

In particular, prodrugs of the present invention include those compounds of formula (a) wherein particular groups below form prodrug functions—i.e. the upper hydroxy group and the radical R¹, wherein such R¹ group is OR⁷ or NR⁸R⁹. The formation of prodrug functions may be accomplished by esterifying the hydroxy groups, or by making amides from the amine NR⁸R⁹ function. Examples of esters include amongst other, oxalic acid ethyl ester, cyclopropane carboxylic acid ester, acetic acid ester, 4-ethoxy-butyric acid ester, hexanoic acid ester, dodecanoic acid ester, hexadecanoic acid ester. In a particular embodiment, both the upper hydroxy group and the R¹ may be transformed into 2 prodrug moieties in the same molecule.

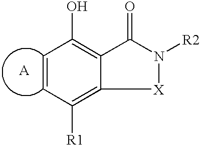

For therapeutic use, the salts of the compounds of the present invention are those wherein the counter-ion is pharmaceutically or physiologically acceptable. However, salts having a pharmaceutically unacceptable counter-ion may also find use, for example, in the preparation or purification of a pharmaceutically acceptable compound of the present invention. All salts, whether pharmaceutically acceptable or not are included within the ambit of the present invention.

The pharmaceutically acceptable or physiologically tolerable addition salt forms which the compounds of the present invention are able to form can conveniently be prepared using the appropriate acids, such as, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Conversely said acid addition salt forms can be converted by treatment with an appropriate base into the free base form.

The compounds of the present invention containing an acidic proton may also be converted into their non-toxic metal or amine addition salt form by treatment with appropriate organic and inorganic bases. Appropriate base salt forms comprise, for example, the ammonium salts, quaternary ammonium salts, the alkali and earth alkaline metal salts, e.g. the lithium, sodium, potassium, magnesium, calcium salts and the like, salts with organic bases, e.g. the benzathine, N-methyl, -D-glucamine, hydrabamine salts, and salts with amino acids such as, for example, arginine, lysine and the like.

Conversely said base addition salt forms can be converted by treatment with an appropriate acid into the free acid form.

The term “salts” also comprises the hydrates and the solvent addition forms that the compounds of the present invention are able to form. Examples of such forms are e.g. hydrates, alcoholates and the like.

The N-oxide forms of the present compounds are meant to comprise the compounds wherein one or several nitrogen atoms are oxidized to the so-called N-oxide.

The present compounds may also exist in their tautomeric forms. Such forms, although not explicitly indicated in the above formula are intended to be included within the scope of the present invention.

The term stereochemically isomeric forms of compounds of the present invention, as used hereinbefore, defines all possible compounds made up of the same atoms bonded by the same sequence of bonds but having different three-dimensional structures which are not interchangeable, which the compounds of the present invention may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of the present invention both in pure form and in admixture with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term ‘stereoisomerically pure’ concerns compounds or intermediates having a stereoisomeric excess of at least 80% (i.e. minimum 80% of one isomer and maximum 20% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms ‘enantiomerically pure’ and ‘diastereomerically pure’ should be understood in a similar way, but then having regard to the enantiomeric excess, respectively the diastereomeric excess of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of compounds of the present invention can be obtained separately by conventional methods. Appropriate physical separation methods which may advantageously be employed are, for example, selective crystallization and chromatography, e.g. column chromatography.

The compounds may contain an asymmetric center and thus may exist as different stereoisomeric forms. Stereoisomeric forms may occur when for instance R³ is different from R⁴. Examples of asymmetric centers are indicated with an asterisk (*) in the structures below.

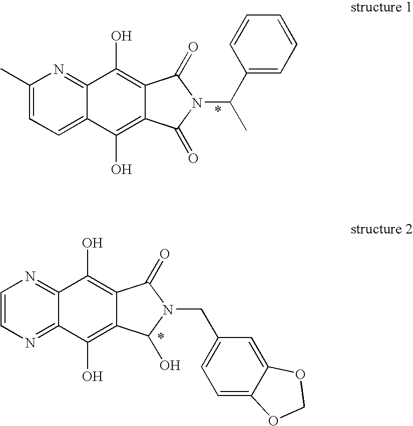

The absolute configuration of each asymmetric center that may be present in the compounds may be indicated by the stereochemical descriptors R and S, this R and S notation corresponding to the rules described in Pure Appl. Chem. 1976, 45, 11-30.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

Interesting compounds are those compounds of formula (I) or any subgroup thereof as defined herein or combination of such subgroups, wherein A together with the two carbons of the phenyl ring to which it is attached forms (i) a phenyl ring optionally substituted with one or more substituents independently selected from halogen, nitro, oxo, cyano, C₃₋₇cycloalkyl, Het¹, Het², C(═O)—R⁸, S(═O)_{y}—R¹⁴, OR⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₄alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴; or (ii) a 5 or 6-membered aromatic heterocycle consisting of at least two carbon atoms and one or two nitrogen atoms, which heterocycle may optionally be substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₄alkynyl and optionally polysubstituted phenyl; whereby the optional substituents on C₁₋₆alkyl, C₂₋₆-alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴.

Other interesting compounds are those compounds of formula (1) or any subgroup thereof as defined herein or combination of such subgroups, wherein A together with the two carbons of the phenyl ring to which it is attached forms a 5 or 6-membered aromatic heterocycle consisting of at least two carbon atoms and one or two nitrogen or sulfur atoms, which heterocycle may optionally be substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴.

Particularly interesting compounds are those compounds of formula (I) or any subgroup thereof as defined herein or combination of such subgroups, wherein A together with the two carbons of the phenyl ring to which it is attached forms (i) a phenyl ring optionally substituted with one substituent selected from halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; or (ii) a pyridinyl, a imidazolyl or a pyrazinyl each of which heterocycle may optionally be substituted on a carbon atom or where possible a nitrogen atom with one substituent selected from halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₄alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

Other particularly interesting compounds are those compounds of formula (I) or any subgroup thereof as defined herein or combination of such subgroups, wherein A together with the two carbons of the phenyl ring to which it is attached forms a pyridinyl, a pyrimidinyl, a imidazolyl, a thiazolyl, a pyrazinyl, or a pyridazinyl each of which heterocycle may optionally be substituted on a carbon atom or where possible a nitrogen atom with one substituent selected from halogen, OR¹⁴, N¹⁴R¹⁴, or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

More particularly interesting compounds are those compounds of formula (I) or any subgroup thereof as defined herein or combination of such subgroups, wherein A together with the two carbons of the phenyl ring to which it is attached forms (i) a phenyl ring optionally substituted with one substituent selected from halogen or optionally substituted C₁₋₆alkyl; whereby the optional substituent on C₁₋₆alkyl is selected from phenyl or OR¹⁴; or (ii) a pyridinyl or a pyrazinyl each of which heterocycle may optionally be substituted on a carbon atom with one substituent selected from halogen or optionally substituted C₁₋₆alkyl; whereby the optional substituent on C₁₋₆alkyl is selected from phenyl or OR¹⁴; or (iii) an imidazolyl optionally substituted on a nitrogen atom with optionally substituted C₁₋₆alkyl; whereby the optional substituent on C₁₋₆alkyl is selected from phenyl or OR¹⁴.

Other more particularly interesting compounds are those compounds of formula (I) or any subgroup thereof as defined herein or combination of such subgroups, wherein A together with the two carbons of the phenyl ring to which it is attached forms (i) a pyridinyl or a pyrazinyl each of which heterocycle may optionally be substituted on a carbon atom with one substituent selected from OR¹⁴ or optionally substituted C₁₋₆alkyl; whereby the optional substituent on C₁₋₆alkyl is selected from phenyl or OR¹⁴; or (ii) an imidazolyl optionally substituted on a nitrogen atom with optionally substituted C₁₋₆alkyl; whereby the optional substituent on C₁₋₆alkyl is selected from phenyl or OR¹⁴.

Another interesting group of compounds are those compounds of formula (I) or any subgroup thereof as defined herein or combination of such subgroups, wherein R¹ is C(═O)—R⁵, S(═O)_{y}—R⁶, OR⁷, NR⁸R⁹, optionally polysubstituted C₂₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆-alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹.

Particularly interesting compounds are those compounds of formula (I) or any subgroup thereof as defined herein or combination of such subgroups, wherein R¹ is OR⁷, optionally substituted C₁₋₆allyl, optionally substituted C₂₋₆alkenyl or optionally substituted C₂₋₆alkynyl; whereby the optional substituent on C₁₋₆alkyl, C₂₋₆alkenyl and C₂₋₆alkynyl is OR⁷.

More particularly interesting compounds are those compounds of formula (I) or any subgroup thereof as defined herein or combination of such subgroups, wherein R¹ is OR⁷.

Even more particularly interesting compounds are those compounds of formula (I) or any subgroup thereof as defined herein or combination of such subgroups, wherein R¹ is OR⁷; whereby R⁷ is hydrogen, C(—O)—R¹⁰, S(═O)_{y}—R¹¹, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from halogen, nitro, cyano, C₃₋₇cycloalkyl, aryl, Het¹, Het², C(═O)—R¹⁰, S(═O)_{y}—R¹¹, OR¹², and NR⁸R¹³.

Yet even more particularly interesting compounds are those compounds of formula (I) or any subgroup thereof as defined herein or combination of such subgroups, wherein R¹ is OR⁷; whereby R⁷ is hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from C₃₋₇cycloalkyl, aryl, Het¹, Het², and preferably is aryl.

Other particularly interesting groups are those compounds of formula (I) or any subgroup thereof as defined herein or combination of such subgroups, wherein R¹ is NR⁸R⁹, and whereby R⁸ is hydrogen or C₁₋₆alkyl, and R⁹ is selected from aryl, C₃₋₇cycloalkyl, Het¹, Het², C(═O)—R¹⁰, S(═O)_{y}—R¹¹, and C₁₋₆alkyl.

Another interesting group of compounds are those compounds of formula (I) or any subgroup thereof as defined herein or combination of such subgroups, wherein R² is hydrogen, C₃₋₇cycloalkyl, aryl, Het¹, Het², or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹.

Another interesting group of compounds are those compounds of formula (1) or any subgroup thereof as defined herein or combination of such subgroups, wherein R² is hydrogen, C₃₋₅cycloalkyl, C₇cycloalkyl, aryl, Het¹, Het², C₂₋₆alkyl or polysubstituted C₁₋₆alkyl, optionally polysubstituted C₂₋₆alkenyl or optionally polysubstituted C₂₋₆alkynyl; whereby the substituents on C₁₋₆alkyl, and the optional substituents on C₂₋₆alkenyl and C₂₋₆alkynyl are each independently selected from halogen, nitro, cyano, C₃₋₇cycloalkyl, aryl, Het¹, Het², C(═O)—R⁵, S(—O)_{y}—R⁶, OR⁷, and NR⁸R⁹.

Particularly interesting compounds are those compounds of formula (I) or any subgroup thereof as defined herein or combination of such subgroups, wherein R² is hydrogen, C₃₋₇cycloalkyl, aryl, Het¹, Het², or optionally substituted C₁₋₆alkyl; whereby the optional substituent on C₁₋₆alkyl is selected from halogen, nitro, cyano, C₃₋₇cycloalkyl, aryl, Het¹, Het², C(═O)—R⁵, S(═O)_{y}—R⁶, OR⁷, and NR⁸R⁹.

More particularly interesting compounds are those compounds of formula (I) or any subgroup thereof as defined herein or combination of such subgroups, wherein R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

Even more particularly interesting compounds are those compounds of formula (I) or any subgroup thereof as defined herein or combination of such subgroups, wherein R² is hydrogen, C₃₋₅cycloalkyl, C₇cycloalkyl, aryl, Het¹, Het², C₂₋₆alkyl, or polysubstituted C₁₋₆alkyl; whereby the substituent on C₁₋₆alkyl is selected from C₃₋₇cycloalkyl, aryl, Het¹, Het², and preferably is C₃₋₇cycloakyl, aryl, Het¹.

Preferred compounds are those compounds of formula (I) or any subgroup thereof as defined herein or combination of such subgroups, wherein R² is 7-benzo[1,3]dioxol-5-ylmethyl, 1-phenyl-ethyl, phenethyl, 3-bromo-benzyl, 3-fluoro-benzyl, 3-chloro-benzyl, 4-bromo-benzyl, 4-fluoro-benzyl, 4-methyloxy-benzyl, 3,4-dichloro-benzyl, 2-cyano-ethyl-benzyl.

Another interesting group of compounds are those compounds of formula (I) or any subgroup thereof as defined herein or combination of such subgroups, wherein X is —C(═O)—.

Another interesting group of compounds are those compounds of formula (I) or any subgroup thereof as defined herein or combination of such subgroups, wherein R⁵ or R¹⁰ is C(═O)—R⁸, C(═O)—OR⁸, C(═O)—NR⁸R⁸, OR⁸, O—C(═O)—R⁸, O—S(═O)_{y}—R⁸, NR⁸R⁸, NR⁸—C(═O)—R⁸, C₁₋₆alkyl, C₂₋₆alkenyl or C₂₋₆alkynyl; or both R⁵ and R¹⁰ are C(═O)—R⁸, C(═O)—OR⁸, C(═O)—NR⁸R⁸, OR⁸, O—C(═O)—R⁸, O—S(—O)_{y}—R⁸, NR⁸R⁸, NR⁸—C(═O)—R⁸, C₁₋₆alkyl, C₂₋₆alkenyl or C₂₋₆alkynyl.

Particularly interesting compounds are those compounds of formula (I) or any subgroup thereof as defined herein or combination of such subgroups, wherein R⁵ or R¹⁰ is C(═O)—R⁸, C(═O)—OR⁸, C(═O)—NR⁸R⁸, OR⁸, NR⁸R⁸, C₁₋₆alkyl; or both R⁵ and R¹⁰ are C(═O)—R⁸, C(═O)—OR⁸, C(—O)—NR⁸R⁸, OR⁸, NR⁸R⁸, C₁₋₆alkyl.

Another interesting group of compounds are those compounds of formula (I) or any subgroup thereof as defined herein or combination of such subgroups, wherein R⁶ or R¹¹ is aryl, OR⁸, NR⁸R⁸, C₁₋₆alkyl; or both R⁶ and R¹¹ are aryl, OR⁸, NR⁸R⁸, C₁₋₆alkyl.

Another interesting group of compounds are those compounds of formula (I) or any subgroup thereof as defined herein or combination of such subgroups, wherein R⁷ or R¹² is hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from halogen, nitro, cyano, C₃₋₇cycloalkyl, aryl, Het¹, Het², C(═O)—R¹⁰, S(═O)_{y}—R¹¹, OR¹², and NR⁸R¹³; or both R⁷ and R¹² are hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from halogen, nitro, cyano, C₃₋₇cycloalkyl, aryl, Het¹, Het², C(═O)—R¹⁰, S(═O)_{y}—R¹¹, OR¹², and NR⁸R¹³.

Particularly interesting compounds are those compounds of formula (I) or any subgroup thereof as defined herein or combination of such subgroups, wherein R⁷ or R¹² is hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from C₃₋₇cycloalkyl, aryl, Het¹, Het², and preferably is aryl; or both R⁷ and R¹² are hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from C₃₋₇cycloalkyl, aryl, Het¹, Het², and preferably is aryl.

Another interesting group of compounds are those compounds of formula (I) or any subgroup thereof as defined herein or combination of such subgroups, wherein R¹⁴ is hydrogen, phenyl, C₁₋₆alkyl, C₃₋₇cycloalkyl.

Another interesting group of compounds are those compounds of formula (I) or any subgroup thereof as defined herein or combination of such subgroups, wherein R⁸ is hydrogen or C₁₋₆alkyl.

Another interesting group of compounds are those compounds of formula (I) or any subgroup thereof as defined herein or combination of such subgroups, wherein R⁹ is hydrogen, aryl, Het¹, Het², C(═O)—R¹⁰, optionally polysubstituted C₁₋₆alkyl; whereby the optional substituents on C₁₋₆alkyl are each independently selected from halogen, nitro, cyano, C₃₋₇cycloalkyl, aryl, Het¹, Het², C(═O)—R¹⁰, S(═O)_{y}—R¹¹, OR¹² and NR⁸R¹³.

Particularly interesting compounds are those compounds of formula (I) or any subgroup thereof as defined herein or combination of such subgroups, wherein R⁹ is hydrogen, aryl, C(═O)—R¹⁰, optionally substituted C₁₋₆alkyl; whereby the optional substituent on C₁₋₆alkyl is selected from halogen, nitro, cyano, C₃₋₇cycloalkyl, aryl, Het¹, Het², C(═O)—R¹⁰, S(═O)_{y}—R¹¹, OR¹² and NR⁸R¹³.

More particularly interesting compounds are those compounds of formula (I) or any subgroup thereof as defined herein or combination of such subgroups, wherein R⁹ is hydrogen or C₁₋₆alkyl.

A special group of compounds are those compounds of formula (I) wherein
A together with the two carbons of the phenyl ring to which it is attached forms (i) a phenyl ring optionally substituted with one substituent selected from halogen or optionally substituted C₁₋₆alkyl; whereby the optional substituent on C₁₋₆alkyl is selected from phenyl or OR¹⁴; or (ii) a pyridinyl or a pyrazinyl each of which heterocycle may optionally be substituted on a carbon atom with one substituent selected from halogen or optionally substituted C₁₋₆alkyl; whereby the optional substituent on C₁₋₆alkyl is selected from phenyl or OR¹⁴; or (iii) an imidazolyl optionally substituted on a nitrogen atom with optionally substituted C₁₋₆alkyl; whereby the optional substituent on C₁₋₆alkyl is selected from phenyl or OR¹⁴;
R¹ is OR⁷; R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹;
X is —C(═O)—;
R⁷ is hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alynyl is selected from C₃₋₇cycloalkyl, aryl, Het¹, Het², and preferably is aryl;
R¹⁰ is C(═O)—R⁸, C(═O)—OR⁸, C(═O)—NR⁸R⁸, OR⁸, NR⁸R⁸, C₁alkyl;
R¹⁴ is hydrogen, phenyl, C₁₋₄alkyl, C₃₋₇cycloalkyl.

Another special group of compounds are those compounds of formula (I) wherein
A together with the two carbons of the phenyl ring to which it is attached forms (i) a pyridinyl or a pyrazinyl each of which heterocycle may optionally be substituted on a carbon atom with one substituent selected from halogen or optionally substituted C₁₋₆alkyl; whereby the optional substituent on C₁₋₆alkyl is selected from phenyl or OR¹⁴; or (ii) an imidazolyl optionally substituted on a nitrogen atom with optionally substituted C₁₋₆alkyl; whereby the optional substituent on C₁₋₆alkyl is selected from phenyl or OR¹⁴;
R¹ is OR⁷;
R² is hydrogen, C₃₋₅cycloalkyl, C₇cycloalkyl, aryl, Het¹, Het², C₂₋₆alkyl or polysubstituted C₁₋₆alkyl; whereby the substituent on C₁₋₆alkyl is selected from C₃₋₇cycloalkyl, aryl, Het¹, Het², and preferably is C₃₋₇cycloalkyl, aryl, Het¹;
X is —C(═O)—;
R⁷ is hydrogen, C(═O)—R¹⁰, optionally substituted C₁₋₆alkyl, optionally substituted C₂₋₆alkenyl or optionally substituted C₂₋₆alkynyl; whereby the optional substituent on C₁₋₆alkyl, C₂₋₆alkenyl and C₂₋₆alkynyl is selected from C₃₋₇cycloalkyl, aryl, Het¹, Het², and preferably is aryl;
R¹⁰ is C(═O)—R⁸, C(═O)—OR⁸, C(═O)—NR⁸R⁸, OR⁸, NR⁸R⁸, C₁₋₆alkyl; and
R¹⁴ is hydrogen, phenyl, C₁₋₄alkyl, C₃₋₇cycloalkyl.

Suitably and where possible, any of the subgroups defined herein may be further restricted by
X is —C(═O)—;
R¹ is —OR⁷;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

A particular subgroup of the compounds of the present invention is defined by formula (IIa): 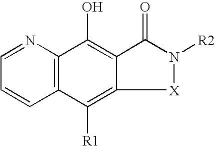
whereby
the pyridinyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

Another particular subgroup of the compounds of the present invention is defined by formula (IIa): 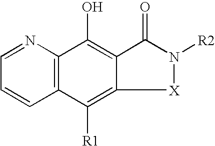
whereby
the pyridinyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴;
   and whereby
R² is not 3,5-dichlorophenyl, nor cyclohexyl, nor methyl.

Suitably, the compounds of formula (IIa) may further be limited to those compounds wherein
X is —C(═O)—;
R¹ is —OR⁷;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

Also suitably, the compounds of formula (IIa) may further be limited to those compounds wherein
X is —C(═O)—;
R¹ is —R⁷;
R² is hydrogen, C₃₋₅cycloalkyl, C₇cycloalkyl, aryl, Het¹, Het², C₂₋₆alkyl or substituted C₁₋₆alkyl; whereby the substituent on C₁₋₆alkyl is selected from C₃₋₇cycloalkyl, aryl, Het¹, Het², and preferably is C₃₋₇cycloalkyl, aryl, Het¹; and whereby R² is not 3,5-dichlorophenyl,

Another particular subgroup of the compounds of the present invention is defined by formula (IIb): 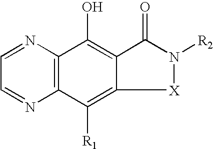
whereby the pyrazinyl ring may optionally be substituted with halogen or optionally polysubstituted C₁alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

Suitably, the compounds of formula (IIb) may further be limited to those compounds wherein
X is —C(═O)—;
R¹ is —OR⁷;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

Another more particular subgroup of the compounds of the present invention is defined by formula (IIc): 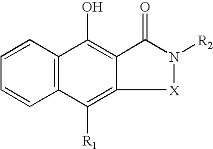
whereby the phenyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(—O)-Het², and NR¹⁴R¹⁴.

Yet another more particular subgroup of the compounds of the present invention is defined by formula (IIc): 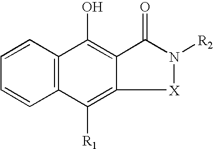
whereby the phenyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby R² is not hydrogen, methyl, cyclohexyl, nor phenyl.

Suitably, the compounds of formula (IIc) may further be limited to those compounds wherein
X is —C(═O)—;
R¹ is —OR⁷;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

Also suitably, the compounds of formula (IIc) may further be limited to those compounds wherein
X is —C(═O)—;
R¹ is —OR⁷;
R² is C₃₋₅cycloalkyl, C₇cycloalkyl, aryl, Het¹, Het², C₂₋₆alkyl or polysubstituted C₁₋₆alkyl; whereby the substituent on C₁₋₆alkyl is selected from C₃₋₇cycloalkyl, aryl, Het¹, Het², and preferably is C₃₋₇cycloalkyl, aryl, Het¹; and whereby R² is not 3,5-dichlorophenyl.

Another more particular subgroup of the compounds of the present invention is defined by formula (IId): 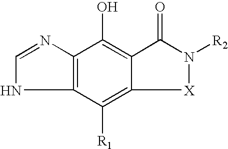
whereby the imidazolyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

Suitably, the compounds of formula (IId) may further be limited to those compounds wherein
X is —C(═O)—;
R¹ is —OR⁷;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

The compounds of formula (IIa), (IIb), (IIc) and (IId) jointly form the compounds of formula (II).

An interesting subgroup within the definition of aryl are the fused bicyclic carbocycles in which one ring is a benzene ring and the other ring is saturated or unsaturated, with attachment via any carbon atom that results in a stable compound. Representative examples of this subset include 1,2,3,4-tetrahydro-naphthalenyl, 1,2-dihydro-naphthalenyl, naphthalenyl, indanyl, 1H-indenyl, bicyclo[4.2.0]octa-1,3,5-trienyl, 6,7,8,9-tetrahydro-5H-benzocycloheptenyl, 6,7-dihydro-5H-benzocycloheptenyl.

Another interesting subgroup within the definition of aryl are the fused tricyclic carbocycles in which one or two rings are a benzene ring and the other ring or rings are saturated or unsaturated, with attachment via any carbon atom that results in a stable compound. Representative examples include, 9H-fluorenyl, anthracenyl, 9,10-dihydro-anthracenyl, 2-phenyl-naphthalenyl, 2-phenyl-1,2,3,4-tetrahydro-naphthalenyl.

An interesting subgroup within the definition of Het¹ are those heterocycles having 5 to 10 ring members, preferably 5 to 8 ring members, more preferably 5 to 6 ring members.

An interesting subgroup within the definition of Het² are those heterocycles having 5 to 10 ring members, preferably 5 to 6 ring members.

A particularly interesting subgroup within the definition of Het¹ and Het² is piperidinyl, piperazinyl, azepinyl, pyrrolyl, pyrrolidinyl, pyrazolyl, pyrazolidinyl, imidazolyl, imidazolidinyl, triazolyl, tetrazolyl, imidazolinyl, pyridyl (also named pyridinyl), pyrazinyl, pyrimidinyl, pyridazinyl, oxazolyl, oxazolidinyl, isoxazolyl, isoxazolidinyl, morpholinyl, thiomorpholinyl, thiazolyl, thiazolidinyl, isothiazolyl, quinoxazolinyl, isothiazolidinyl, quinolinyl, pyrrolyl, thiazolyl, imidazolyl, isooxazolyl, thiadiazolyl, isoquinolinyl, benzimidazolyl, thiadazolyl, benzopyranyl, benzothiazolyl, benzoazolyl, furyl (also named furanyl), tetrahydrofuryl (also named tetrahydrofuranyl), tetrahydro-puranyl, thienyl, benzothienyl, oxadiazolyl, and benzo-1,3-dioxacyclopentyl (also named 1,3-benzodioxolyl), tetrahydrothienyl, tetrahydrodioxothienyl, thiadiazinanyl, dioxothiadiazinanyl, thiazinanyl, dioxothiazinanyl, dioxothiazolidinyl, and isodioxothiazolidinyl, indolyl, benzotriazolyl, imidazo[4,5-b]pyridinyl, dihydroimidazo[4,5-b]-pyridinyl, pyrazolo[4,3-c]pyridinyl, dihydropyrazolo[4,3-c]pyridinyl, tetrahydropyrazolo[4,3-c]pyridinyl, pyrrolo[1,2-a]pyrazinyl, dihydropyrrolo[1,2-a]pyrazinyl, tetrahydropyrrolo[1,2-a]pyrazinyl, octahydropyrrolo[1,2-a]pyrazinyl, isoindolyl, indazolyl, indolinyl, isoindolinyl, quinoxalinyl, quinazolinyl, cinnolinyl, chromanyl, isochromanyl, phthalazinyl, purinyl, 1,6-naphthyridinyl, 1,8-napthyridinyl, dihydroindolyl, dihydroisoindolyl, tetrahydroquinolyl, tetrahydroisoquinolyl, imidazo[1,2-a]pyrimidinyl, 2,3-dihydroimidazo[2,1-b][1,3]thiazolyl, benzazepinyl, dihydrobenazepinyl, benzodiazepinyl, dihydrobenzodiazepinyl, and tetrahydrobenzodiazepinyl, phenothiazinyl, carbazolyl, beta-carbolinyl, tetrahydrobetacarbolinyl, acridinyl, phenazinyl, phenoxazinyl.

A particularly interesting subgroup within the definition of Het¹ and Het² is defined by a fused bicyclic Het¹ or Het² wherein one ring is a benzene ring and the other is a saturated or unsaturated heteroatom-containing ring, more in particular 3,4-dihydro-2H-benzo[1,4]oxazinyl, 2,3-dihydro-1H-benzoimidazolyl, 2,3-dihydro-1H-indolyl, 2,3-dihydro-1H-isoindolyl, 1H-indazolyl, benzooxazoyl, quinolinyl, isoquinolinyl, 4,5-dihydro-3H-benzo[b]azepinyl, 5H-benzo[e][1,4]-diazepinyl, 2,5-dihydro-1H-benzo[b][1,4]diazepinyl, 2,3,4,5-tetrahydro-1H-benzo[b][1,4]diazepinyl, 2,3,4,5-tetrahydro-1H-benzo[b]azepinyl, benzo[1,3]dioxolyl.

A particular subgroup of compounds are those compounds of formula (I) wherein one or more of the following restrictions apply:
X is 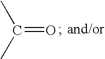
R¹ is hydroxy; O—C₁₋₆-alkanediyl-aryl; O—C₁₋₆alkanediyl-cyano; O—C₁₋₆alkyl; O—C(═O)—C(═O)—O—C₁₋₄alkyl; and/or
R² is aryl, C₃₋₇cycloalkyl, Het¹, Het², C₁₋₆alkanediyl-C₃₋₇cycloalkyl, C₁₋₆alkanediyl-aryl, C₁₋₆alkanediyl-Het¹, C₁₋₆alkanediyl-Het², wherein the C₃₋₇cycloalkyl, aryl, Het¹, or Het² may be optionally substituted on one or more carbons or heteroatoms with halogen, C₁₋₄alkyl, O—C₁₋₄alkyl, S(═O)₂—C₁₋₄alkyl, O-aryl; and/or
the A-ring may be unsubstituted or substituted on one or more carbons or heteroatoms with halogen, C₁₋₄alkyl, C₁₋₄alkanediyl-phenyl.

A more particular subgroup of the compounds of the present invention is defined by formula (III):

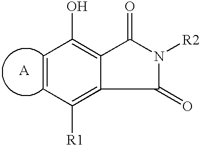

Suitably, the compounds of formula (III) may further be limited to those compounds wherein
R¹ is OR⁷; more in particular hydroxy or O—C₁₋₄alkyl;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

Suitably, the compounds of formula (III) may further be limited to those compounds wherein
A, together with the two carbons of the phenyl ring to which it is attached forms a monocyclic Het²;
R¹ is —OR⁷; more in particular hydroxy or O—C₁₋₄alkyl;
R² is hydrogen, C₃₋₅cycloalkyl, C₇cycloalkyl, aryl, Het¹, Het², C₂₋₆alkyl or substituted C₁₋₆alkyl; whereby the substituent on C₁₋₆alkyl is selected from C₃₋₇cycloalkyl, aryl, Het¹, Het², and preferably is C₃₋₇cycloalkyl, aryl, Het¹; and whereby R² is not 3,5-dichlorophenyl.

The compounds of the present invention can generally be prepared using procedures analogous to those procedures described in the examples.

Particular reaction procedures to make the present compounds are described below. In the preparations described below, the reaction products may be isolated from the medium and, if necessary, further purified according to methodologies generally known in the art such as, for example, extraction, crystallization, trituration and chromatography.

A strategy for a synthesis path of the compounds of the present invention is preparing on one hand a dialkyl-dicarboxylated A-ring, substituted on positions x and y, where y=x+1; preparing on the other hand, a N—R² substituted succinimide; and reacting by means of a double Claisen condensation the methyldicarboxylated A-ring with the N—R² substituted succinimide. The derived products may be optionally reduced, further substituted or experiment other reactions as required.

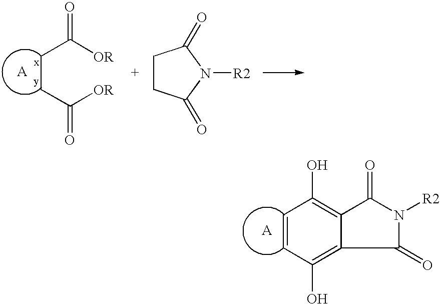

The x,y-dialkyl-dicarboxylated A-ring may be the esterification result of dissolving a x,y-aryldicarboxylic acid with an alcohol, catalyzed with mineral acids and heated. Sulfuric acid, hydrogen chloride, or other known catalysts may be employed as mineral acid catalysts. Alternatively, reacting a salt of x,y-aryldicarboxylate, e.g. sodium x,y-aryldicarboxylate with an haloalkane, in the presence of x,y-aryldicarboxylic acid and heating.

On a parallel scheme, the N—R² substituted succinimide may be obtained by reacting a N—R² substituted amine with succinic anhydride. Said reaction may be enhanced with the addition of suitable solvents, such as acetic acid, in the presence of catalysts like 4-dimethylaminopyridine (MAP). Alternatively, products with solvent and nucleophilic catalyst functions could as well be employed, such as the pyridine-type solvents. N—R² substituted succinimide are as well obtained by combining succinimides with haloalkyls, or haloalkanediyl-aryls in the presence of strong base and solvents.

The amino equivalent, may be obtained by reacting an A-ring substituted with a carboxylate and a cyano group, with a N—R² substituted succinimide.

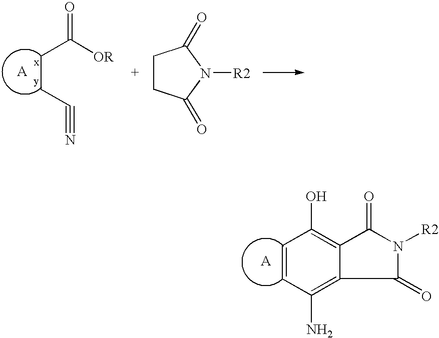

A different strategy for a synthesis may for instance start from a A-ring fused with a cyclic anhydride, followed by a reduction to obtain a lactone, opening the lactone with a sodium thiolate, formation of an amide and oxidation of the sulfide into a sulfoxide with oxidizing agents such as sodium periodate, applying a Pummerer rearrangement, with subsequent Diels-Alder and elimination cascade in a one-pot-procedure to yield the compounds of this invention.

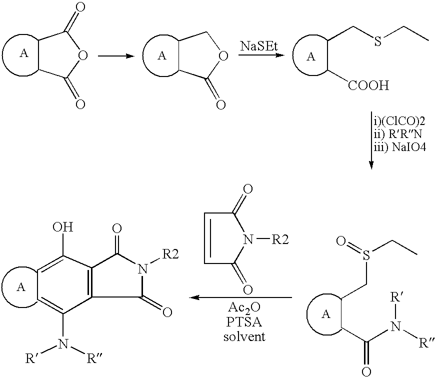

Reduction of the cyclic anhydride to obtain a lactone is achieved by treating with a reducing agent, optionally in the presence of an acid. Examples are available in the literature and include for instance reducing a quinolinic anhydride with NaBH₄ in the presence of AcOH to obtain a furopyridinone.

In an alternative route of synthesis of compounds of the present invention, an A-ring, wherein x—alkyl-carboxylate, and y=2-methyl-[1,3]dioxolan-2-yl, is reacted by means of a Claisen condensation with a N—R² substituted succinimide catalyzed by mineral acids such as sodium hydride. Subsequent contact with an acid, preferably a strong acid, releases the cyclic group leaving an acyl group, which in the presence of a mineral acid, yields compounds of the formula (III), wherein R¹ is an alkyl group, as indicated in the scheme below:

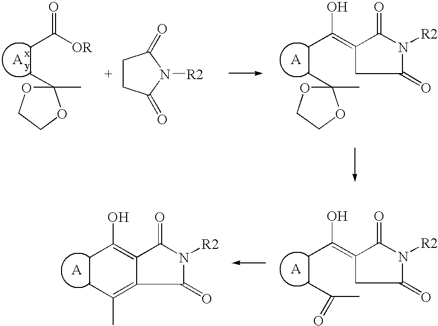

The derived products may be optionally reduced, further substituted or experiment other reactions. For instance, when X is an oxo group, such may be converted into dimethyl by following the synthesis encompassed in reference Tetrahedron, 57(13), 2581-2588; 2001. Optionally, the X=oxo group may be converted into 2 radicals: R and hydroxyl by means of a Grignard reaction, as here under illustrated:

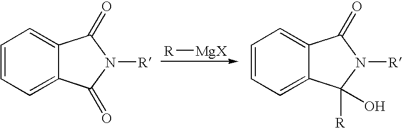

In addition, X as oxo group may be converted into a diphenyl moiety by reacting as here under illustrated:

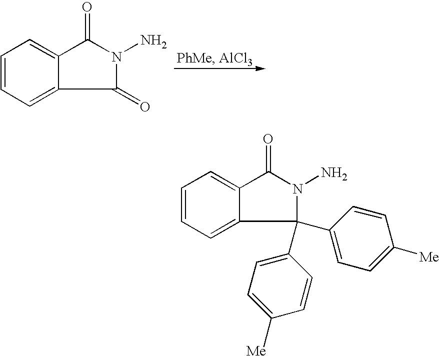

Reference: Heterocycles, 46, 225-233; 1997;

Eventually the X=oxo moiety may be transformed into a thiooxo group through thionation with Lawesson's reagent (e.g. Synthesis 1996, 1485-1488).

Conversion of the X=oxo group into an amino, may be performed as follows:

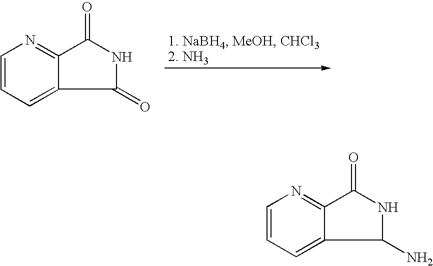

Reference: Bulletin of the Chemical Society of Japan, 60(11), 4178-80; 1987.

Further, by means of a Wittig reaction, an X=alkenediyl moiety is obtained from the oxo group.

Alternatively, one may obtain a ring closure through a double Claisen condensation on:

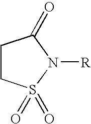

By reduction of the monothionated compound with Raney Nickel, one can obtain a X=—CH₂-moiety.

Variants of the R¹ group may be obtained as indicated below in the table:

| target R¹ | |
|---|---|
| moiety is | synthesis starting from the hydroxyl or amino group |
| hydrogen | by hydrogenating the triflate with Pd/C in a |
| | suitable solvent |
| halogen | by chlorinating the phenol with SOCl2 or POCl3 |
| nitro | through nitration of the parent phenol. |
| sultam | by reacting the triflate or bromoderivative with |
| | 2H-1,2-Thiazine, tetrahydro-, 1,1-dioxide in the |
| | presence of a suitable copper catalyst. |
| C₃₋₇cycloalkyl | By a Heck reaction with a cycloalkene on the |
| | triflate followed by a hydrogenation |
| C(═O)—R | by a Diels Alder on the parent isobenzofurane system |
| S(═O)ₓ—R | by a Diels Alder on the parent isobenzofurane system |
| OR⁷ | by alkylation of the parent phenol. Already 1 example |
| | described |
| C(═NR⁸)—R | by a Diels Alder on the parent isobenzofurane system |
| C₁₋₆alkyl | through a Stille coupling |
| C₂₋₆alkenyl | through a Stille coupling |
| C₂₋₆alkynyl | by a Sonogashira reaction |

Alternatively when R¹ is a hydroxyl group, introduction of a toluenesulfonyl group may be accomplished with for instance TsCl, and the use of a base such as triethylamine in the presence of an appropriate solvent such as dichloromethane.

In another embodiment, introduction of a R′-carboxylic acid ester at the R¹ hydroxy group may be accomplished by reacting the hydroxy moiety with the R′-carboxylic acid, a coupling reagent such as TBTU (2-(1H-benzotriazol-1-yl)-1,1,3,3-tetramethyluronium tetrafluoroborate), a base such as triethylaine in the presence of the appropriate solvents and reactions conditions.

In another embodiment, introduction of a Het¹ group into R¹, wherein the Het¹ is for instance is a pyrrolidona, and the nitrogen is the point of attachment to the benzene group of the core-structure, may be accomplished by reacting the 5-Amino-7-(3-bromo-benzyl)-9-hydroxy-pyrrolo[3,4-g]quinoline-6,8-dione with dihydro-furan-2,5-dione dissolved in the appropriate solvents and in the presence of a catalytic amount of reagents as employed in acylation reactions, e.g. DMAP. Alternatively, when the Het¹ group introduced as R¹ is a pyrrol, said moiety may be obtained by reacting 5-Amino-7-(3-bromo-benzyl)-9-hydroxy-pyrrolo[3,4-g]quinoline-6,8-dione with and 2,5-dimethoxy-tetrahydro-furan dissolved in the appropriate solvents.

To introduce a m-halobenzyl as a R² moiety, N-benzylmaleimide can be prepared by treating maleic anhydride with m-halobenzylamine to give N-halobenzylmaleamic acid, which is treated with anhydride NaOAc and anhydride HOAc at around 80° C.

To introduce C₁₋₄alkanediyl-aryl-C₁₋₄alkyl as a R² moiety, one may follow the next reaction:

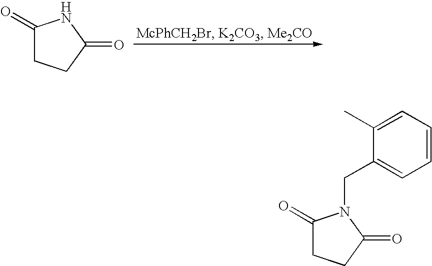

Alternatively, in the reaction above, reagent MePhCH₂Br may include a Het¹ or Het² groups instead of the phenyl group, by which C₁₋₄alkanediyl-Het¹-C₁₋₄alkyl, and C₁₋₄alkanediyl-Het²-C₁₋₄alkyl could be inserted as R² moieties.

To introduce C₃₋₇cycloalkyl, or C₁₋₄alkanediyl-C₃₋₇cycloalkyl, as R² moieties, as example, cyclohexylamine and maleic anhydride would be reacted at 100° C. in O-xylene to give a slurry of N-cyclohexyl maleamic acid to which a slurry of dicyclohexylamine salt of H₂SO₄ would be added and the mixture heated at 147° C. for 2 h with azeotropic H₂O removal to give N-cyclohexylmaleimide of high purity. Alternatively, N-cyclohexylmethylamine could be employed to obtain the corresponding N-cyclohexylmethyl)maleimide.

For the introduction of C₁₋₄alkanediyl-aryl-O-aryls as R² moieties, the artisan may obtain those from commercially available compounds such as,

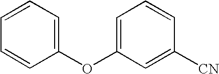

CAS 50789-45-2, available at Apin Chemical Ltd., and then convert them by a Raney-Nichel reaction into their amino equivalents, followed by a reaction with succinic anhydride to form:

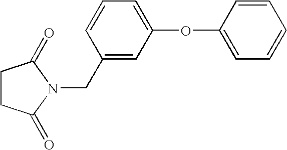

Het¹ as R² moiety, may be for instance obtained from commercial sources, such as Interchim Intermediates, CAS 170805-72-8:

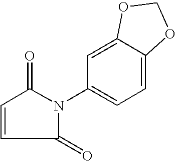

Het² as R² moiety, may be for instance obtained from commercial sources, such as Interbioscreen Compound Library, CAS 69971-90-0:

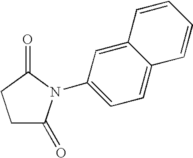

Pyrazole, as A-ring, may be transformed into its corresponding maleic anhydride by placing 2-diazoketones in reaction with maleic anhydride and MeCOCHN2.

For preparation of 2,3-quinolinedicarboxylic acid, the artisan may follow the syntheis as disclosed in reference Bull. Soc. Chel. Belg., 89, nr. 3, 1980, pg 205, or alternatively in reference by OPPI, 14, 396, (82).

Indole with 2 carboxylic groups is commercially available from SALOR.

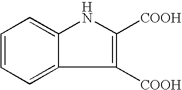

Synthesis of pyridazine-3,4-dicarboxylic acid may be achieved by a hetero Diels-Alder reaction as disclosed in Journal of Heterocylic Chemistry (1990), 27(3), 579-82.

Reactive pyrroles may be obtained as follows:

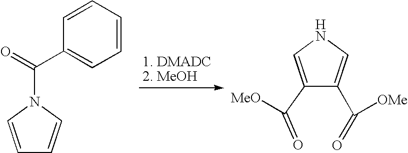

Similarly, reactive furane is obtained by:

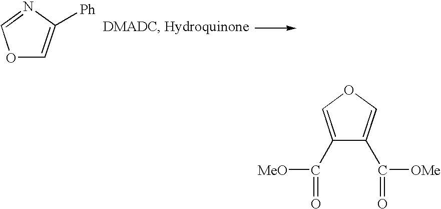

1H-1,2,3-Triazole-4,5-dicarboxylic acid, dimethyl is commercially available from ChemDiv, Inc. Product Library.

Reactive benzofurane is obtained for instance:

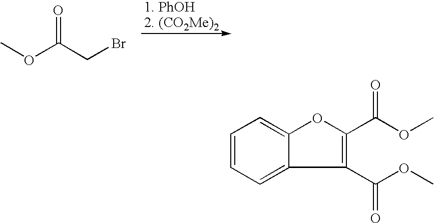

HCNO, prepared in situ by hydrolysis of Me₂SiCNO in aqueous THF, may undergo cycloaddition reactions with alkenes and alkynes to give isoxazoles.

Isothiazole may become reactive with the introduction of the 2 carboxylate moieties as follows:

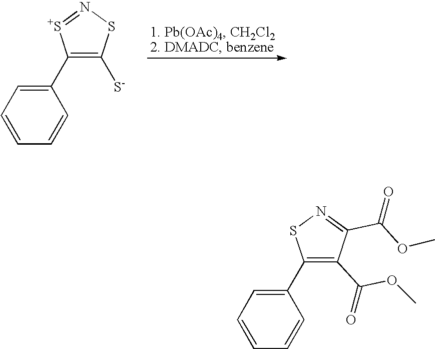

A thiophene with 2 carboxylate moieties may be obtained from:

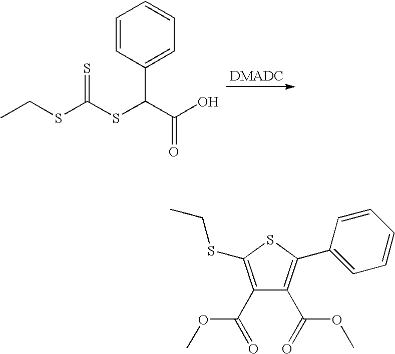

A carbazole with 2 carboxylate groups is obtained from:

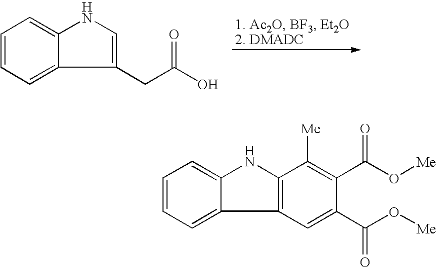

Reactive quinoxalines may be prepared as follows:

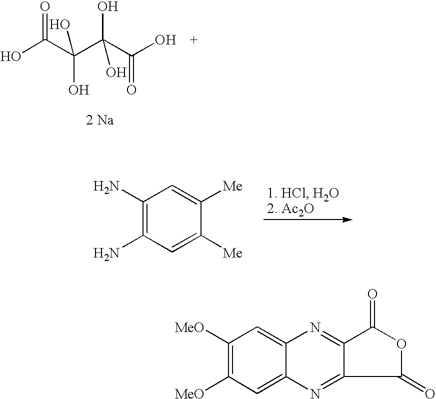

The compounds of the present invention may also be converted to the corresponding N-oxide forms following art-known procedures for converting a trivalent nitrogen into its N-oxide form. Said N-oxidation reaction may generally be carried out by reacting the starting material of compounds with appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chloro-benzenecarboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

The present compounds can thus be used in animals, preferably in mammals, and in particular in humans as pharmaceuticals per se, in mixtures with one another or in the form of pharmaceutical preparations.

Furthermore, the present invention relates to pharmaceutical preparations which as active constituents contain an effective dose of at least one of the compounds of this invention in addition to customary pharmaceutically innocuous excipients and auxiliaries. The pharmaceutical preparations normally contain 0.1 to 90% by weight of the compound. The pharmaceutical preparations can be prepared in a manner known per se to one of skill in the art. For this purpose, at least one of a compound of this invention, together with one or more solid or liquid pharmaceutical excipients and/or auxiliaries and, if desired, in combination with other pharmaceutical active compounds, are brought into a suitable administration form or dosage form which can then be used as a pharmaceutical in human medicine or veterinary medicine.

Pharmaceuticals which contain a compound according to the invention can be administered orally, parenterally, e.g., intravenously, rectally, by inhalation, or topically, the preferred administration being dependent on the individual case, e.g., the particular course of the disorder to be treated. Oral administration is preferred.

The person skilled in the art is familiar on the basis of his expert knowledge with the auxiliaries which are suitable for the desired pharmaceutical formulation. Beside solvents, gel-forming agents, suppository bases, tablet auxiliaries and other active compound carriers, antioxidants, dispersants, emulsifiers, antifoams, flavor corrigents, preservatives, solubilizers, agents for achieving a depot effect, buffer substances or colorants are also useful.

The compounds of the present invention are useful in the treatment of individuals infected by HIV and for the prophylaxis of these individuals. In general, the compounds of the present invention may be useful in the treatment of warm-blooded animals infected with viruses whose existence is mediated by, or depends upon, the integrase enzyme. Conditions which may be prevented or treated with the compounds of the present invention, especially conditions associated with HIV and other pathogenic retroviruses, include AIDS, AIDS-related complex (ARC), progressive generalized lymphadenopathy (PGL), as well as chronic CNS diseases caused by retroviruses, such as, for example HIV mediated dementia and multiple sclerosis.

The compounds of the present invention or any subgroup thereof may therefore be used as medicines against above-mentioned conditions. Said use as a medicine or method of treatment comprises the systemic administration to HIV-infected subjects of an amount effective to combat the conditions associated with HIV and other pathogenic retroviruses, such as HIV-1. Consequently, the compounds of the present invention can be used in the manufacture of a medicament useful for treating conditions associated with HIV and other pathogenic retroviruses.

In a preferred embodiment, the invention relates to the use of a compound of formula (I), (II), i.e. (IIa), (IIb), (IIc), and (IId), and (III) or any subgroup thereof in the manufacture of a medicament for treating or combating infection or disease associated with retrovirus infection in a mammal, such as HIV-1 infection. Thus, the invention also relates to a method of treating a retroviral infection, or a disease associated with retrovirus infection comprising administering to a mammal in need thereof an effective amount of a compound of formula (I), (II) and (II) or a subgroup thereof.

In another preferred embodiment, the present invention relates to the use of compound of this invention in the manufacture of a medicament for inhibiting a integrase of a retrovirus in a mammal infected with said retrovirus, in particular HIV-1 retrovirus.

In another preferred embodiment, the present invention relates to the use of compounds of this invention in the manufacture of a medicament for inhibiting retroviral integration, in particular HIV-1 integration.

The compounds of the present invention may also find use in inhibiting ex vivo samples containing HIV or expected to be exposed to HIV. Hence, the present compounds may be used to inhibit HIV present in a body fluid sample which contains or is suspected to contain or be exposed to HIV.

Also, the combination of an antiretroviral compound and a compound of the present invention can be used as a medicine. Thus, the present invention also relates to a product containing (a) a compound of the present invention, and (b) another antiretroviral compound, as a combined preparation for simultaneous, separate or sequential use in treatment of retroviral infections. Thus, to combat or treat HIV infections, or the infection and disease associated with HIV infections, such as Acquired Immunodeficiency Syndrome (AIDS) or AIDS Related Complex (ARC), the compounds of this invention may be co-administered in combination with for instance, binding inhibitors, such as, for example, dextran sulfate, suramine, polyanions, soluble CD4; fusion inhibitors, such as, for example, T20, T1249, SHC-C; co-receptor binding inhibitors, such as, for example, AMD 3100 (Bicyclams), TAK 779; RT inhibitors, such as, for example, foscarnet and prodrugs; nucleoside RTIs, such as, for example, AZT, 3TC, ddC, ddI, d4T, abacavir, FTC, DAPD, dOTC; nucleotide RTIs, such as, for example, PMEA, PMPA, tenofovir; NNRTIs, such as, for example, nevirapine, delavirdine, efavirenz, 8 and 9-Cl TIBO (tivirapine), loviride, TMC-125, TMC-120, MKC-442, UC 781, capravirine, DPC 961, DPC963, DPC082, DPC083, calanolide A, SJ-3366, TSAO, 4″-deaminated TSAO; RNAse H inhibitors, such as, for example, SP1093V, PD126338; TAT inhibitors, such as, for example, RO-5-3335, K12, K37; integrase inhibitors, such as, for example, L 708906, L 731988; protease inhibitors, such as, for example, amprenavir, ritonavir, nelfinavir, saquinavir, indinavir, lopinavir, lasinavir, BMS 232632, BMS 186316, DPC 681, DPC 684, tipranavir, AG1776, DMP 450, L 756425, PD178390, PNU 140135; glycosylation inhibitors, such as, for example, castanospermine, deoxynojirimycine.

The combination may provide a synergistic effect, whereby viral infectivity and its associated symptoms may be prevented, substantially reduced, or eliminated completely.

The compounds of the present invention may also be administered in combination with immunomodulators (e.g., bropirimine, anti-human alpha interferon antibody, IL-2, methionine enkephalin, interferon alpha, and naltrexone) or with antibiotics (e.g., pentamidine isothiorate) to ameliorate, combat, or eliminate HIV infection and its symptoms.

For an oral administration form, compounds of the present invention are mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms.

For subcutaneous or intravenous administration, the active compounds, if desired with the substances customary therefor such as solubilizers, emulsifiers or further auxiliaries, are brought into solution, suspension, or emulsion. The compounds can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned.

Suitable pharmaceutical formulations for administration in the form of aerosols or sprays are, for example, solutions, suspensions or emulsions of the compounds of the present invention, or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Such a preparation customarily contains the active compound in a concentration from approximately 0.1 to 50%, in particular from approximately 0.3 to 3% by weight.

In order to enhance the solubility and/or the stability of the compounds in pharmaceutical compositions, it can be advantageous to employ α-, β- or γ-cyclo-dextrins or their derivatives. Also co-solvents such as alcohols may improve the solubility and/or the stability of the compounds in pharmaceutical compositions. In the preparation of aqueous compositions, addition salts of the subject compounds are obviously more suitable due to their increased water solubility.

Appropriate cyclodextrins are α-, β- or γ-cyclodextrins (CDs) or ethers and mixed ethers thereof wherein one or more of the hydroxy groups of the anhydroglucose units of the cyclodextrin are substituted with C₁₋₆alkyl-, particularly methyl, ethyl or isopropyl, e.g. randomly methylated β-CD; hydroxyC₁₋₆alkyl-, particularly hydroxyethyl, hydroxypropyl or hydroxybutyl; carboxyC₁₋₆alkyl-, particularly carboxymethyl or carboxyethyl; C₁₋₆alkylcarbonyl-, particularly acetyl; C₁₋₆alkyloxycarbonylC₁₋₆alkyl- or carboxyC₁₋₆alkyloxyC₁₋₆alkyl-, particularly carboxymethoxypropyl or carboxyethoxypropyl; C₁₋₆alkylcarbonyloxyC₁₋₆alkyl-, particularly 2-acetyloxypropyl. Especially noteworthy as complexants and/or solubilizers are β-CD, randomly methylated β-CD, 2,6-dimethyl-β-CD, 2-hydroxy-ethyl-β-CD, 2-hydroxyethyl-γ-CD, 2-hydroxypropyl-γ-CD and (2-carboxymethoxy)-propyl-β-CD, and in particular 2-hydroxypropyl-β-CD (2-HP-β-CD).

The term mixed ether denotes cyclodextrin derivatives wherein at least two cyclodextrin hydroxy groups are etherified with different groups such as, for example, hydroxy-propyl and hydroxyethyl.

An interesting way of formulating the present compounds in combination with a cyclodextrin or a derivative thereof has been described in EP-A-721,331. Although the formulations described therein are with antifungal active ingredients, they are equally interesting for formulating the compounds of the present invention. The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition. Said formulations may also be rendered more palatable by adding pharmaceutically acceptable sweeteners and/or flavors.

Other convenient ways to enhance the solubility of the compounds of the present invention in pharmaceutical compositions are described in WO-94/05263, PCT application No. PCT/EP98/01773, EP-A-499299 and WO 97/44014, all incorporated herein by reference.

More in particular, the present compounds may be formulated in a pharmaceutical composition comprising a therapeutically effective amount of particles consisting of a solid dispersion comprising (a) a compound of the present invention, and (b) one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

The term “a solid dispersion” also comprises dispersions which are less homogenous throughout than solid solutions. Such dispersions are not chemically and physically uniform throughout or comprise more than one phase.

The water-soluble polymer in the particles is conveniently a polymer that has an apparent viscosity of 1 to 100 mPa·s when dissolved in a 2% aqueous solution at 20° C. solution.

Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule.

The particles as defined hereinabove can be prepared by first preparing a solid dispersion of the components, and then optionally grinding or milling that dispersion. Various techniques exist for preparing solid dispersions including melt-extrusion, spray-drying and solution-evaporation.

It may further be convenient to formulate the present compounds in the form of nanoparticles which have a surface modifier adsorbed on the surface thereof in an amount sufficient to maintain an effective average particle size of less than 1000 nm. Useful surface modifiers are believed to include those which physically adhere to the surface of the antiretroviral agent but do not chemically bond to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the present compounds involves a pharmaceutical composition whereby the present compounds are incorporated in hydrophilic polymers and applying this mixture as a coat film over many small beads, thus yielding a composition with good bioavailability which can conveniently be manufactured and which is suitable for preparing pharmaceutical dosage forms for oral administration.

Said beads comprise (a) a central, rounded or spherical core, (b) a coating film of a hydrophilic polymer and an antiretroviral agent and (c) a seal-coating polymer layer.

Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

Another aspect of the present invention concerns a kit or container comprising a compound of the present invention, in an amount effective for use as a standard or reagent in a test or assay for determining the ability of a potential pharmaceutical to inhibit HIV integrase, HIV growth, or both. This aspect of the invention may find its use in pharmaceutical research programs.

The compounds of the present invention can be used in phenotypic resistance monitoring assays, such as known recombinant assays, in the clinical management of resistance developing diseases such as HIV. A particularly useful resistance monitoring system is a recombinant assay known as the Antivirogram™. The Antivirogram™ is a highly automated, high throughput, second generation, recombinant assay that can measure susceptibility, especially viral susceptibility, to the compounds of the present invention. (Fertogs K, de Bethune M P, Miller V et al. Antimicrob Agents Chemother, 1998; 42(2): 269-276, incorporated by reference).

The dose of the present compounds or of the physiologically tolerable salt(s) thereof to be administered depends on the individual case and, as customary, is to be adapted to the conditions of the individual case for an optimum effect. Thus it depends, of course, on the frequency of administration and on the potency and duration of action of the compounds employed in each case for therapy or prophylaxis, but also on the nature and severity of the infection and symptoms, and on the sex, age, weight and individual responsiveness of the human or animal to be treated and on whether the therapy is acute or prophylactic. Customarily, the daily dose of a compound of the present invention, in the case of administration to a patient approximately 75 kg in weight is 1 mg to 1 g, preferably 3 mg to 0.5 g. The dose can be administered in the form of an individual dose, or divided into several, e.g. two, three, or four, individual doses.

The following table lists compounds of this invention, which were prepared following one of the above reaction schemes.

TABLE 1

| | |
|---|---|
| Compound 1 | 7-Benzo[1,3]dioxol-5-ylmethyl-5,9-dihydroxy-pyrrolo[3,4-g]quinoxaline-6,8-dione |
| Compound 2 | 7-(4-Fluoro-benzyl)-5,9-dihydroxy-pyrrolo[3,4-g]quinoxaline-6,8-dione |
| Compound 3 | 5,9-Dihydroxy-7-(4-methyl-benzyl)-pyrrolo[3,4-g]quinoxaline-6,8-dione |
| Compound 4 | 7-(4-Bromo-benzyl)-5,9-dihydroxy-pyrrolo[3,4-g]quinoxaline-6,8-dione |
| Compound 5 | 7-Benzo[1,3]dioxol-5-ylmethyl-5,9-dihydroxy-pyrrolo[3,4-g]quinoline-6,8-dione |
| Compound 6 | Oxalic acid 7-benzo[1,3]dioxol-5-ylmethyl-9-hydroxy-6,8-dihydro-6H- |
| | pyrrolo[3,4-g]quinoxalin-5-yl ester ethyl ester |
| Compound 7 | 5,9-Dihydroxy-7-(4-phenoxy-benzyl)-pyrrolo[3,4-g]quinoxaline-6,8-dione |
| Compound 8 | 5,9-Dihydroxy-2-methyl-7-(1-phenyl-ethyl)-pyrrolo[3,4-g]quinoline-6,8-dione |
| Compound 9 | 2-Benzo[1,3]dioxol-5-yl-4,9-dihydroxy-benzo[f]isoindole-1,3-dione |
| Compound 10 | 7-Benzo[1,3]dioxol-5-yl-5,9-dihydroxy-pyrrolo[3,4-g]quinoxaline-6,8-dione |
| Compound 11 | 2-(4-Fluoro-benzyl)-4,9-dihydroxy-benzo[f]isoindole-1,3-dione |
| Compound 12 | 4,9-Dihydroxy-2-phenethyl-benzo[f]isoindole-1,3-dione |
| Compound 13 | 5-Fluoro-2-(4-fluoro-benzyl)-4,9-dihydroxy-benzo[f]isoindole-1,3-dione |
| Compound 14 | 5,9-Dihydroxy-7-phenethyl-pyrrolo[3,4-g]quinoxaline-6,8-dione |
| Compound 15 | 5,9-Dihydroxy-7-(1-(R)-phenyl-ethyl)-pyrrolo[3,4-g]quinoxaline-6,8-dione |
| Compound 16 | 7-(3-Fluoro-benzyl)-5,9-dihydroxy-pyrrolo[3,4-g]quinoxaline-6,8-dione |
| Compound 17 | 5,9-Dihydroxy-7-(4-methoxy-benzyl)-pyrrolo[3,4-g]quinoxaline-6,8-dione |
| Compound 18 | 7-[3-(5,9-Dihydroxy-6,8-dioxo-6,8-dihydro-pyrrolo[3,4-g]quinoxalin-7-ylmethyl)- |
| | benzyl]-5,9-dihydroxy-pyrrolo[3,4-g]quinoxaline-6,8-dione |
| Compound 19 | 5,9-Dihydroxy-2-methyl-7-phenethyl-pyrrolo[3,4-g]quinoline-6,8-dione |
| Compound 20 | 7-Benzo[1,3]dioxol-5-ylmethyl-5,9-dihydroxy-2-methyl-pyrrolo[3,4-g]quinoline-6,8- |
| | dione |
| Compound 21 | 7-(3-Fluoro-benzyl)-5,9-dihydroxy-2-methyl-pyrrolo[3,4-g]quinoline-6,8-dione |
| Compound 22 | 7-(4-Fluoro-benzyl)-5,9-dihydroxy-2-methyl-pyrrolo[3,4-g]quinoline-6,8-dione |
| Compound 23 | 5,9-Dihydroxy-7-(4-methoxy-benzyl)-2-methyl-pyrrolo[3,4-g]quinoline-6,8-dione |
| Compound 24 | 5,9-Dihydroxy-7-phenethyl-pyrrolo[3,4-g]quinoline-6,8-dione |
| Compound 25 | 5,9-Dihydroxy-7-(1-phenyl-ethyl)-pyrrolo[3,4-g]quinoxaline-6,8-dione |
| Compound 26 | 7-Cyclohexylmethyl-5,9-Dihydroxy-pyrrolo[3,4-g]quinoxaline-6,8-dione |
| Compound 27 | 5,9-Dihydroxy-7-naphthalen-1-ylmethyl-pyrrolo[3,4-g]quinoxaline-6,8-dione |
| Compound 28 | 5,9-Dihydroxy-7-(2-morpholine-4-yl-ethyl)-pyrrolo[3,4-g]quinoxaline-6,8-dione |
| Compound 29 | 5,9-Dihydroxy-7-pyridin-4-ylmethyl-pyrrolo[3,4-g]quinoxaline-6,8-dione |
| Compound 30 | 5,9-Dihydroxy-2-methyl-7-phenyl-pyrrolo[3,4-g]quinoline-6,8-dione |
| Compound 31 | 7-Benzo[1,3]dioxol-5-ylmethyl-5-benzyloxy-9-hydroxy-pyrrolo[3,4-g]quinoxaline-6,8- |
| | dione |
| Compound 32 | 7-Cyclopentyl-5,9-dihydroxy-pyrrolo[3,4-g]quinoxaline-6,8-dione |
| Compound 33 | 7-(4-Fluoro-phenyl)-5,9-dihydroxy-pyrrolo[3,4-g]quinoxaline-6,8-dione |
| Compound 34 | 5,9-Dihydroxy-7-(4-methanesulfonyl-benzyl)-pyrrolo[3,4-g]quinoxaline-6,8-dione |
| Compound 35 | 7-(2-Bromo-phenyl)-5,9-dihydroxy-pyrrolo[3,4-g]quinoxaline-6,8-dione |
| Compound 36 | 7-Benzo[1,3]dioxol-5-ylmethyl-5-hydroxy-9-prop-2-ynyloxy-pyrrolo[3,4- |
| | g]quinoxaline-6,8-dione |
| Compound 37 | 7-Benzo[1,3]dioxol-5-ylmethyl-5-hydroxy-9-(3-methyl-butoxy)-pyrrolo[3,4- |
| | g]quinoxaline-6,8-dione |
| Compound 38 | 6-Benzo[1,3]dioxol-5-ylmethyl-1-benzyl-4,8-dihydroxy-1H-1,3,6-triaza-s-indacene- |
| | 5,7-dione |
| Compound 39 | 6-Benzo[1,3]dioxol-5-ylmethyl-4,8-dihydroxy-1H-1,3,6-triaza-s-indacene-5,7-dione |
| Compound 40 | 1-Benzyl-4,8-dihydroxy-6-(1-phenyl-ethyl)-1H-1,3,6-triaza-s-indacene-5,7-dione |
| Compound 41 | 7-Benzo[1,3]dioxol-5-ylmethyl-5-hydroxy-9-(3-phenyl-propoxy)-pyrrolo[3,4- |
| | g]quinoxaline-6,8-dione |
| Compound 42 | 4,9-Dihydroxy-2-(1-phenyl-ethyl)-benzo[f]isoindole-1,3-dione |
| Compound 43 | 7-Benzo[1,3]dioxol-5-ylmethyl-5-hydroxy-pyrrolo[3,4-g]quinoxaline-6,8-dione |
| Compound 44 | 7-Benzhydryl-5,9-dihydroxy-pyrrolo[3,4-g]quinoxaline-6,8-dione |
| Compound 45 | 5,9-Dihydroxy-7-(5-phenyl-1H-pyrazol-3-yl)-pyrrolo[3,4-g]quinoxaline-6,8-dione |
| Compound 46 | 2-(5,9-Dihydroxy-6,8-dioxo-6,8-dihydro-pyrrolo[3,4-g]quinoxalin-7-ylmethyl)- |
| | benzonitrile |
| Compound 47 | 5,9-Dihydroxy-7-(4′-phenoxy-biphenyl-4-ylmethyl)-pyrrolo[3,4-g]quinoxaline-6,8- |
| | dione |
| Compound 48 | 5-(Benzyl-methyl-amino)-7-(3-bromo-benzyl)-9-hydroxy-pyrrolo[3,4-g]quinoline-6,8- |
| | dione |
| Compound 49 | 7-(2′-Chloro-biphenyl-3-ylmethyl)-5,9-dihydroxy-pyrrolo[3,4-g]quinoxaline-6,8-dione |
| Compound 50 | 6-(3-Bromo-benzyl)-4,8-dihydroxy-2-methyl-thiazolo[4,5-e]isoindole-5,7-dione |
| Compound 51 | 1-(3,5-Dichloro-phenyl)-3-[3-(5,9-dihydroxy-6,8-dioxo-6,8-dihydro-pyrrolo[3,4- |
| | g]quinoxalin-7-ylmethyl)-phenyl]-urea |
| Compound 52 | 3′-(5,9-Dihydroxy-6,8-dioxo-6,8-dihydro-pyrrolo[3,4-g]quinoxalin-7-ylmethyl)- |
| | biphenyl-4-carboxylic acid amide |
| Compound 53 | 3′-(5,9-Dihydroxy-6,8-dioxo-6,8-dihydro-pyrrolo[3,4-g]quinoxalin-7-ylmethyl)- |
| | biphenyl-4-carbonitrile |
| Compound 54 | 5-Amino-7-(3-bromo-benzyl)-9-hydroxy-pyrrolo[3,4-g]quinoline-6,8-dione |
| Compound 55 | 5,9-Dihydroxy-7-(3-pyridin-3-yl-benzyl)-pyrrolo[3,4-g]quinoxaline-6,8-dione |
| Compound 56 | 3-[3-(5,9-Dihydroxy-6,8-dioxo-6,8-dihydro-pyrrolo[3,4-g]quinoxalin-7-ylmethyl)- |
| | phenyl]-acrylonitrile |
| Compound 57 | 3-[3-(5,9-Dihydroxy-6,8-dioxo-6,8-dihydro-pyrrolo[3,4-g]quinoxalin-7-ylmethyl)- |
| | phenyl]-N,N-dimethyl-propionamide |
| Compound 58 | N-(7-Benzo[1,3]dioxol-5-ylmethyl-9-hydroxy-6,8-dioxo-7,8-dihydro-6H-pyrrolo[3,4- |
| | g]quinolin-5-yl)-methanesulfonamide |
| Compound 59 | 5,9-Dihydroxy-7-(3-methylamino-benzyl)-pyrrolo[3,4-g]quinoxaline-6,8-dione |
| Compound 60 | [4-(5,9-Dihydroxy-6,8-dioxo-6,8-dihydro-pyrrolo[3,4-g]quinoxalin-7-ylmethyl)- |
| | phenyl]-carbamic acid methyl ester |
| Compound 61 | 7-[2-(2,5-Dioxo-pyrrolidin-1-yl)-1,2-diphenyl-ethyl]-5,9-dihydroxy-pyrrolo[3,4- |
| | g]quinoxaline-6,8-dione |
| Compound 62 | Acetic acid 9-acetoxy-7-benzo[1,3]dioxol-5-ylmethyl-6,8-dioxo-7,8-dihydro-6H- |
| | pyrrolo[3,4-g]quinoxalin-5-yl ester |
| Compound 63 | 7-(2-Benzo[1,3]dioxol-5-yl-ethyl)-5,9-dihydroxy-pyrrolo[3,4-g]quinoxaline-6,8-dione |
| Compound 64 | 5,9-Dihydroxy-7-(4-iodo-benzyl)-pyrrolo[3,4-g]quinoxaline-6,8-dione |
| Compound 65 | 4,8-Dihydroxy-6-(1-phenyl-ethyl)-1H-1,3,6-triaza-s-indacene-5,7-dione |
| Compound 66 | 6-Benzo[1,3]dioxol-5-ylmethyl-4,8-dihydroxy-1-(2-hydroxy-ethyl)-1H-1,3,6-triaza-s- |
| | indacene-5,7-dione |
| Compound 67 | 7-(4-Chloro-benzyl)-5,9-dihydroxy-pyrrolo[3,4-g]quinoxaline-6,8-dione |
| Compound 68 | 7-(3-Bromo-benzyl)-5,9-dihydroxy-pyrrolo[3,4-g]quinoxaline-6,8-dione |
| Compound 69 | 7-(3-Bromo-4-fluoro-benzyl)-5,9-dihydroxy-pyrrolo[3,4-g]quinoxaline-6,8-dione |
| Compound 70 | 7-(4-Bromo-2-fluoro-benzyl)-5,9-dihydroxy-pyrrolo[3,4-g]quinoxaline-6,8-dione |
| Compound 71 | 7-(5-Bromo-2-fluoro-benzyl)-5,9-dihydroxy-pyrrolo[3,4-g]quinoxaline-6,8-dione |
| Compound 72 | 5,9-Dihydroxy-7-(3-trifluoromethoxy-benzyl)-pyrrolo[3,4-g]quinoxaline-6,8-dione |
| Compound 73 | 5,9-Dihydroxy-7-(4-trifluoromethoxy-benzyl)-pyrrolo[3,4-g]quinoxaline-6,8-dione |
| Compound 74 | 7-Benzyl-5-(benzyl-methyl-amino)-9-hydroxy-pyrrolo[3,4-g]quinoline-6,8-dione |
| Compound 75 | Toluene-4-sulfonic acid 7-benzo[1,3]dioxol-5-ylmethyl-9-hydroxy-6,8-dioxo-7,8- |
| | dihydro-6H-pyrrolo[3,4-g]quinoxalin-5-yl ester |
| Compound 76 | 7-Benzo[1,3]dioxol-5-ylmethyl-5-(benzyl-methyl-amino)-9-hydroxy-pyrrolo[3,4- |
| | g]quinoline-6,8-dione |
| Compound 77 | 7-(3-Chloro-benzyl)-5,9-dihydroxy-pyrrolo[3,4-g]quinoxaline-6,8-dione |
| Compound 78 | 7-(3,4-Dichloro-benzyl)-5,9-dihydroxy-pyrrolo[3,4-g]quinoxaline-6,8-dione |
| Compound 79 | 7-(3,5-Dichloro-benzyl)-5,9-dihydroxy-pyrrolo[3,4-g]quinoxaline-6,8-dione |
| Compound 80 | 4-(5,9-Dihydroxy-6,8-dioxo-6,8-dihydro-pyrrolo[3,4-g]quinoxalin-7-ylmethyl)- |
| | benzonitrile |
| Compound 81 | 4,9-Dihydroxy-2-phenethyl-2,5,6-triaza-cyclopenta[b]naphthalene-1,3-dione |
| Compound 82 | 2-Benzo[1,3]dioxol-5-ylmethyl-4,9-dihydroxy-2,5,6-triaza-cyclopenta[b]naphthalene- |
| | 1,3-dione |
| Compound 83 | 7-(4-Bromo-benzyl)-5,9-dihydroxy-pyrrolo[3,4-g]quinoline-6,8-dione |
| Compound 84 | 2-(4-Bromo-benzyl)-4,9-dihydroxy-2,5,6-triaza-cyclopenta[b]naphthalene-1,3-dione |
| Compound 85 | 7-(4-Bromo-benzyl)-5,9-dihydroxy-2-methyl-pyrrolo[3,4-g]quinoline-6,8-dione |
| Compound 86 | 5,9-Dihydroxy-7-(2-hydroxy-2-phenyl-ethyl)-pyrrolo[3,4-g]quinoxaline-6,8-dione |
| Compound 87 | 7-(2-Bromo-benzyl)-5,9-dihydroxy-pyrrolo[3,4-g]quinoxaline-6,8-dione |
| Compound 88 | 7-(2-Chloro-benzyl)-5,9-dihydroxy-pyrrolo[3,4-g]quinoxaline-6,8-dione |
| Compound 89 | 1-Benzyl-6-(3-bromo-benzyl)-4,8-dihydroxy-1H-1,3,6-triaza-s-indacene-5,7-dione |
| Compound 90 | 7-(1H-Benzoimidazol-2-ylmethyl)-5,9-dihydroxy-pyrrolo[3,4-g]quinoxaline-6,8-dione |
| Compound 91 | 2-(3-Bromo-benzyl)-4,9-dihydroxy-2,5,6-triaza-cyclopenta[b]naphthalene-1,3-dione |
| Compound 92 | 7-(3-Bromo-benzyl)-5,9-dihydroxy-2-methyl-pyrrolo[3,4-g]quinoline-6,8-dione |
| Compound 93 | 7-(3-Bromo-benzyl)-5,9-dihydroxy-pyrrolo[3,4-g]quinoline-6,8-dione |
| Compound 94 | 5,9-Dihydroxy-7-(2′-methoxy-bipheneyl-3-ylmethyl)-pyrrolo[3,4-g]quinoxaline-6,8- |
| | dione |
| Compound 95 | 7-(3-Bromo-benzyl)-5-hydroxy-9-(3-phenyl-propoxy)-pyrrolo[3,4-g]quinoxaline-6,8- |
| | dione |
| Compound 96 | 7-(3-Bromo-benzyl)-5-hydroxy-9-isopropoxy-pyrrolo[3,4-g]quinoxaline-6,8-dione |
| Compound 97 | 7-(3-Bromo-benzyl)-5-ethoxy-9-hydroxy-pyrrolo[3,4-g]quinoxaline-6,8-dione |
| Compound 98 | 7-(3-Bromo-benzyl)-5,9-diisopropoxy-pyrrolo[3,4-g]quinoxaline-6,8-dione |
| Compound 99 | 7-(3-Benzo[1,3]dioxol-5-yl-benzyl)-5,9-dihydroxy-pyrrolo[3,4-g]quinoxaline-6,8- |
| | dione |
| Compound 100 | 5,9-Dihydroxy-7-(3-thiophen-2-yl-benzyl)-pyrrolo[3,4-g]quinoxaline-6,8-dione |
| Compound 101 | 7-(3-Bromo-benzyl)-5-hydroxy-9-isobutoxy-pyrrolo[3,4-g]quinoxaline-6,8-dione |
| Compound 102 | 7-(3-Bromo-benzyl)-5-(4-fluoro-benzyloxy)-9-hydroxy-pyrrolo[3,4-g]quinoxaline-6,8- |
| | dione |
| Compound 103 | 7-(3-Bromo-benzyl)-5-cyclopentyloxy-9-hydroxy-pyrrolo[3,4-g]quinoxaline-6,8-dione |
| Compound 104 | 7-(3-Bromo-benzyl)-5-hydroxy-9-(3-methyl-butoxy)-pyrrolo[3,4-g]quinoxaline-6,8- |
| | dione |
| Compound 105 | [7-(3-Bromo-benzyl)-9-hydroxy-6,8-dioxo-7,8-dihydro-6H-pyrrolo[3,4-g]quinoxalin- |
| | 5-yloxy]-acetonitrile |
| Compound 106 | 7-(2′-Fluoro-biphenyl-3-ylmethyl)-5,9-dihydroxy-pyrrolo[3,4-g]quinoxaline-6,8-dione |
| Compound 107 | 5,9-Dihydroxy-7-(4′-methoxy-biphenyl-3-ylmethyl)-pyrrolo[3,4-g]quinoxaline-6,8- |
| | dione |
| Compound 108 | 6-(3-Bromo-benzyl)-4-hydroxy-2,8-dimethyl-thizolo[4,5-e]isoindole-5,7-dione |
| Compound 109 | 7-(3′-Amino-biphenyl-3-ylmethyl)-5,9-dihydroxy-pyrrolo[3,4-g]quinoxaline-6,8-dione |
| Compound 110 | 7-(3-Benzofuran-2-yl-benzyl)-5,9-dihydroxy-pyrrolo[3,4-g]quinoxaline-6,8-dione |
| Compound 111 | 5,9-Dihydroxy-7-methyl-pyrrolo[3,4-g]quinoxaline-6,8-dione |
| Compound 112 | 5,9-Dihydroxy-7-(2′-methoxy-biphenyl-4-ylmethyl)-pyrrolo[3,4-g]quinoxaline-6,8- |
| | dione |
| Compound 113 | 7-(4-Benzo[b]thiophen-2-yl-benzyl)-5,9-dihydroxy-pyrrolo[3,4-g]quinoxaline-6,8- |
| | dione |
| Compound 114 | 7-(2′-Fluoro-biphenyl-4-ylmethyl)5,9-dihydroxy-pyrrolo[3,4-g]quinoxaline-6,8-dione |
| Compound 115 | 7-(4-Benzofuran-2-yl-benzyl)-5,9-dihydroxy-pyrrolo[3,4-g]quinoxaline-6,8-dione |
| Compound 116 | 5,9-Dihydroxy-7-(4-thiophen-2-yl-benzyl)-pyrrolo[3,4-g]quinoxaline-6,8-dione |
| Compound 117 | 8-Amino-6-(3-bromo-benzyl)-4-hydroxy-2-methyl-thiazolo[4,5-e]isoindole-5,7-dione |
| Compound 118 | N-[6-(3-Bromo-benzyl)-4-hydroxy-2-methyl-5,7-dioxo-6,7-dihydro-5H-thiazolo[4,5- |
| | e]isoindol-8-yl]-methanesulfonamide |
| Compound 119 | 5-Amino-7-(3-bromo-benzyl)-9-hydroxy-pyrrolo[3,4-g]quinoline-6,8-dione |
| Compound 120 | N-[7-(3-Bromo-benzyl)-9-hydroxy-6,8-dioxo-7,8-dihydro-6H-pyrrolo[3,4-g]quinolin- |
| | 5-yl]-methanesulfonamide |
| Compound 121 | 7-(1-Benzyl-piperidin-4-yl)-5,9-dihydroxy-pyrrolo[3,4-g]quinoxaline-6,8-dione |
| Compound 122 | 5,9-Dihydroxy-7-pyridin-3-ylmethyl-pyrrolo[3,4-g]quinoxaline-6,8-dione |
| Compound 123 | 3-[3-(5,9-Dihydroxy-6,8-dioxo-6,8-dihydro-pyrrolo[3,4-g]quinoxalin-7-ylmethyl)- |
| | phenyl]-N,N-dimethyl-acrylamide |
| Compound 124 | 7-(1-Benzoyl-piperidin-4-yl)-5,9-dihydroxy-pyrrolo[3,4-g]quinoxaline-6,8-dione |
| Compound 125 | 5,9-Dihydroxy-7-(1-methanesulfonyl-piperidin-4-yl)-pyrrolo[3,4-g]quinoxaline-6,8- |
| | dione |
| Compound 126 | 3-[3-(5,9-Dihydroxy-6,8-dioxo-6,8-dihydro-pyrrolo[3,4-g]quinoxalin-7-ylmethyl)- |
| | phenyl]-acrylonitrile |
| Compound 127 | 7-(2,3-Dimethoxy-benzyl)-5,9-dihydroxy-pyrrolo[3,4-g]quinoxaline-6,8-dione |
| Compound 128 | 7-(2,4-Dimethoxy-benzyl)-5,9-dihydroxy-pyrrolo[3,4-g]quinoxaline-6,8-dione |
| Compound 129 | 4-(5,9-Dihydroxy-6,8-dioxo-6,8-dihydro-pyrrolo[3,4-g]quinoxaline-7-yl)-piperidine-1- |
| | carboxylic acid tert-butyl ester |
| Compound 130 | 5,9-Dihydroxy-7-(2-methoxy-benzyl)-pyrrolo[3,4-g]quinoxaline-6,8-dione |
| Compound 131 | 7-(2,6-Dimethoxy-benzyl)-5,9-dihydroxy-pyrrolo[3,4-g]quinoxaline-6,8-dione |
| Compound 132 | 7-(2,5-Dimethoxy-benzyl)-5,9-dihydroxy-pyrrolo[3,4-g]quinoxaline-6,8-dione |
| Compound 133 | 5,9-Dihydroxy-7-piperdin-4-yl-pyrrolo[3,4-g]quinoxaline-6,8-dione |
| Compound 134 | 5,9-Dihydroxy-7-(3-trifluoromethyl-benzyl)-pyrrolo[3,4-g]quinoxaline-6,8-dione |
| Compound 135 | 7-(3-Amino-benzyl)-5,9-dihydroxy-pyrrolo[3,4-g]quinoxaline-6,8-dione |
| Compound 136 | [4-(5,9-Dihydroxy-6,8-dioxo-6,8-dihydro-pyrrolo[3,4-g]quinoxalin-7-ylmethyl)- |
| | phenyl]-carbamic acid tert-butyl ester |
| Compound 137 | 7-(3-Chloro-4-fluoro-benzyl)-5,9-dihydroxy-pyrrolo[3,4-g]quinoxaline-6,8-dione |
| Compound 138 | 7-(3,5-Difluoro-benzyl)-5,9-dihydroxy-pyrrolo[3,4-g]quinoxaline-6,8-dione |
| Compound 139 | 5,9-Dihydroxy-7-(3-iodo-benzyl)-pyrrolo[3,4-g]quinoxaline-6,8-dione |
| Compound 140 | 7-(3,4-Difluoro-benzyl)-5,9-dihydroxy-pyrrolo[3,4-g]quinoxaline-6,8-dione |
| Compound 141 | 7-(3-Bromo-benzyl)-2-dimethylamino-5,9-dihydroxy-pyrrolo[3,4-g]quinazoline-6,8- |
| | dione |
| Compound 142 | 5,9-Dihydroxy-7-(3,4,5-trifluoro-benzyl)-pyrrolo[3,4-g]quinoxaline-6,8-dione |
| Compound 143 | 7-Benzo[1,3]dioxol-5-ylmethyl-5,9-dihydroxy-2,3-dimethyl-pyrrolo[3,4-g]quinoxaline- |
| | 6,8-dione |
| Compound 144 | 7-(3-Bromo-benzyl)-5,9-dihydroxy-2-methyl-pyrrolo[3,4-g]quinoxaline-6,8-dione |
| Compound 145 | 7-Benzo[1,3]dioxol-5-ylmethyl-5,9-dihydroxy-2-methyl-pyrrolo[3,4-g]quinoxaline- |
| | 6,8-dione |
| Compound 146 | N-(7-Benzo[1,3]dioxol-5-ylmethyl-9-hydroxy-6,8-dioxo-7,8-dihydro-6H-pyrrolo[3,4- |
| | g]quinolin-5-yl)-4-cyano-benzenesulfonamide |
| Compound 147 | 5-Bromo-thiophene-2-sulfonic acid (7-benzo[1,3]dioxol-5-ylmethyl-9-hydroxy-6,8- |
| | dioxo-7,8-dihydro-6H-pyrrolo[3,4-g]quinolin-5-yl)-amide |
| Compound 148 | 2-Benzylamino-7-(3-bromo-benzyl)-5,9-dihydroxy-pyrrolo[3,4-g]quinazoline-6,8- |
| | dione |
| Compound 149 | 7-(3-Bromo-benzyl)-5,9-dihydroxy-2-methoxy-pyrrolo[3,4-g]quinoxaline-6,8-dione |
| Compound 150 | 7-(6-Bromo-2,3-dimethoxy-benzyl)-5,9-dihydroxy-pyrrolo[3,4-g]quinoxaline-6,8- |
| | dione |
| Compound 151 | 7-(2,3-Dibromo-5,6-dimethoxy-benzyl)-5,9-dihydroxy-pyrrolo[3,4-g]quinoxaline-6,8- |
| | dione |
| Compound 152 | Hexanoic acid 7-benzo[1,3]dioxol-5-ylmethyl-9-hydroxy-6,8-dioxo-7,8-dihydro-6H- |
| | pyrrolo[3,4-g]quinoxalin-5-yl ester |
| Compound 153 | 5,9-Dihydroxy-7-[1-(thiophene-2-sulfonyl)-piperidin-3-ylmethyl]-pyrrolo[3,4- |
| | g]quinoxaline-6,8-dione |
| Compound 154 | 7-(1-Benzenesulfonyl-piperidin-3-ylmethyl)-5,9-dihydroxy-pyrrolo[3,4-g]quinoxaline- |
| | 6,8-dione |
| Compound 155 | Hexadecanoic acid 7-(3,4-dichloro-benzyl)-9-hexadecanoyloxy-6,8-dioxo-7,8-dihydro- |
| | 6H-pyrrolo[3,4-g]quinoxalin-5-yl ester |
| Compound 156 | 7-(5-Bromo-2,3-dimethoxy-benzyl)-5,9-dihydroxy-pyrrolo[3,4-g]quinoxaline-6,8- |
| | dione |
| Compound 157 | Hexanoic acid 7-benzo[1,3]dioxol-5-ylmethyl-9-hexanoyloxy-6,8-dioxo-7,8-dihydro- |
| | 6H-pyrrolo[3,4-g]quinoxalin-5-yl ester |
| Compound 158 | Hexanoic acid 7-(3,4-dichloro-benzyl)-9-hexanoyloxy-6,8-dioxo-7,8-dihydro-6H- |
| | pyrrolo[3,4-g]quinoxalin-5-yl ester |
| Compound 159 | Acetic acid 9-acetoxy-7-(3,4-dichloro-benzyl)-6,8-dioxo-7,8-dihydro-6H-pyrrolo[3,4- |
| | g]quinoxalin-5-yl ester |
| Compound 160 | Hexadecanoic acid 7-benzo[1,3]dioxol-5-ylmethyl-9-hexadecanoyloxy-6,8-dioxo-7,8- |
| | dihydro-6H-pyrrolo[3,4-g]quinoxalin-5-yl ester |
| Compound 161 | Dodecanoic acid 7-benzo[1,3]dioxol-5-ylmethyl-9-hydroxy-6,8-dioxo-7,8-dihydro-6H- |
| | pyrrolo[3,4-g]quinoxalin-5-yl ester |
| Compound 162 | Dicyclopropanecarboxylic acid 7-(3,4-dichloro-benzyl)-6,8-dioxo-7,8-dihydro-6H- |
| | pyrrolo[3,4-g]quinoxalin-5,9-diyl ester |
| Compound 163 | 7-(3,4-Dichloro-benzyl)-5,9-dihydroxy-2-methyl-pyrrolo[3,4-g]quinoxaline-6,8-dione |

### EXAMPLE 1

### Preparation of 7-Benzo[1,3]dioxol-5-ylmethyl-5,9-dihydroxy-pyrrolo[3,4-g]-quinoxaline-6,8-dione

### Step 1: Preparation of 2,3-pyrazinemethyldicarboxylate

2,3-pyrazinedicarboxylicacid (21.9 g, 0.13 mol) was dissolved in MeOH (400 ml) and the pH was adjusted to 2 with HCl. This mixture was heated at reflux for 16 hrs. After evaporating MeOH, the residue was co-evaporated two times with toluene and dried in a vacuum oven to furnish 26.03 g 2,3-pyrazinemethyldicarboxylate. MS: [M+H]⁺=197

### Step 2: Preparation of N-benzodioxol-5-ylsuccinimide

Piperonylamine (10.0 g, 0.07 mol), succinic anhydride (6.6 g, 0.07 mol) and a catalytic amount of DMAP were dissolved in HOΛc (150 ml). The mixture was heated at reflux for 64 hrs. After evaporation of HOAc, the solid was dissolved in CH₂Cl₂ and washed with NaHCO₃ and diluted HCl. When the CH₂Cl₂ was removed, the residue was co-evaporated two times with toluene and dried in a vacuum oven to afford a light yellow raw material, which was used as such (13.35 g, 87%).

NMR: 1H (CDCl₃): 6.9 (s, 1H), 6.87 (d, J=7.8 Hz, 1H), 6.71 (d, J=7.78 Hz, 1H), 5.92 (s, 2H), 4.55 (s, 2H), 2.68 (s, 4H).

### Step 3: Preparation of 7-Benzo[1,3]dioxol-5-ylmethyl-5,9-dihdroxy-pyrrolo[3,4-g]-quinoxaline-6,8-dione

2,3-pyrazinemethyldicarboxylate (398 mg, 2.03 mmol) and N-benzodioxol-5-ylsuccinimide (487 mg, 2.09 mmol) were dissolved in THF (40 ml). After NaH (4.92 mmol) and MeOH (3 drops) were carefully added, the suspension was heated at reflux for 16 hrs. When THF was evaporated, the residue was suspended in water and ether. The dark red heterogeneous mixture turns yellow after addition of HCl. This mixture was then vigorously stirred for several hrs. The yellow precipitate was then filtered, washed with some ether and dried in a vacuum oven (406 mg, 55%). MS: [M+H]⁺=366, [M−H]⁻=364

NMR: 1H (DMSO): 11.2 (br.s, 2H), 9.10 (s, 2H), 6.89 (d, J=1.51 Hz, 1H), 6.87 (d, J=8 Hz, 1H), 6.80 (dd, J=8 Hz, J=1.5 Hz, 1H), 5.97 (s, 2H), 4.64 (s, 2H).

### EXAMPLE 2

### Preparation of 5,9-Dihydroxy-2-methyl-7-phenethyl-pyrrolo[3,4-g]quinoline-6,8-dione

### Step 1: Preparation of 6-methyl-2,3-pyridinemethyldicarboxylate

6-methyl-2,3-pyridinedicarboxylic acid (5.1 g, 0.03 mol) was dissolved in MeOH (80 ml) and the pH was adjusted to 2 with HCl/isopropanol 6N. This mixture was heated at reflux for 16 hrs. After evaporating MeOH, the residue was co-evaporated two times with toluene and dried in a vacuum oven to get a yellow solid (5.81 g, 98.7%). MS: [M+H]⁺=210

### Step 2: Preparation of N-phenethylsuccinimide

Phenethylamine (7.0 g, 0.06 mol), succinic anhydride (5.8 g, 0.06 mol) and a catalytic amount of DMAP were dissolved in HOAc (100 ml). Then it was heated at reflux for 64 hrs. After evaporation of HOAc, the solid was dissolved in CH₂Cl₂ and washed with NaHCO₃ and diluted HCl. The solution was dried with MgSO₄, evaporated and the residue was dried in a vacuum oven to afford 11.6 g (98.7%), which was used as such.

### Step 3: Preparation of 5,9-Dihydroxy-2-methyl-7-phenethyl-pyrrolo[3,4-g]quinoline-6,8-dione

6-methyl-2,3-pyridinemethyldicarboxylate (410 mg, 1.96 mmol) and N-phenethyl-succinimide (379 mg, 1.87 mmol) were dissolved in THF (40 ml). After NaH (4.60 mmol) and MeOH (4 drops) were carefully added, the suspension was heated at reflux for 16 hrs. When THF was evaporated, the residue was suspended in water and ether. The dark red heterogeneous mixture turns yellow after addition of HCl. This mixture was then vigorously stirred for several hours. The yellow precipitate was then filtered, washed some ether and dried in a vacuum oven (265 mg, 41%). MS: [M+H]+=349, [M−H]−=347

NMR: 1H (DMSO): 10.60 (s, 1H), 8.57 (d, J=8.55 Hz, 1H), 7.66 (d, J=8.55 Hz, 1H), 7.35-7.15 (m, 5H), 3.79 (t, J=7.35 Hz, 2H), 2.93 (t, J=7.35 Hz, 2H), 2.75 (s, 3H).

### EXAMPLE 3

### Preparation of 6-Benzo[1,3]dioxol-5ylmethyl-4,8-dihydroxy-1H-1,3,6-triaza-s-indacene-5,7-dione

### Step 1: Preparation of 1-Benzyl-1H-imidazole-4,5-dicaroxylic acid dimethyl ester

Imidazole-dicarboxylic acid dimethylester (5.1 g, 0.03 mol) was stirred in MeOH (150 ml). To this suspension, Na (680 mg, 0.03 mol) was added. When all solids were dissolved benzylbromide (4.8 g, 0.02 mol) was added, the mixture was heated at reflux for 16 hours. After evaporating MeOH, the residue was dissolved in CH₂Cl₂ and stirred with silica-gel. When the silica was removed by filtration and the solvent was evaporated, the solid was purified over a short silica column to afford the title compound (8.84 g, 75.9%). MS: [M+H]+=275

### Step 2: Preparation of N-benzodioxol-5-ylsuccinimide

The same synthesis route was used as in step 2 of example 1.

### Step 3: Preparation of 6-Benzo[1,3]dioxol-5ylmethyl-1-benzyl-4,8-dihydroxy-1H-1,3,6-triaza-s-indacene-5,7-dione

The compound from step 1 (725 mg, 2.65 mmol) and N-benzodioxol-5-ylmethyl-succinimide (616 mg, 2.64 mmol) were dissolved in THF. After NaH (8.00 mmol) and MeOH (4 drops) were carefully added, the suspension was heated at reflux for 16 hours. When THF was evaporated, the residue was suspended in water and ether. The dark red heterogeneous mixture turns yellow after addition of HCl pH=±5 to 6). This mixture was then vigorously stirred for several hours. The yellow precipitate was then filtered, washed with some ether and dried in a vacuum oven (1.15 g, 98.1%). MS: [M+H]+=444

### Step 4: Preparation of 6-Benzo[1,3]dioxol-5ylmethyl-4,8-dihydroxy-1H-1,3,6-triaza-s-indacene-5,7-dione

A mixture of product obtained in Step 3 (0.88 g, 2.60 mmol), 0.3 g Pd/C (10%), 100 ml MeOH and 3 ml Et3N was hydrogenated for 48 hours. After filtration and evaporation, the title compound was obtained (237 mg, 33%). MS: [M+H]+=354, [M-H]-=352

### EXAMPLE 4

### Preparation of 7-biphenyl-4-ylmethyl-5,9-dihydroxy-pyrrolo[3,4-g]quinoxaline-6,8-dione

### Step 1: Preparation of 2,3-pyrazinemethyldicarboxylate

2,3-pyrazinedicarboxylicacid (21.9 g, 0.13 mol) was dissolved in MeOH (400 ml) and the pH was adjusted to 2 with HCl. This mixture was heated at reflux for 16 hours. After evaporating MeOH, the residue was co-evaporated two times with toluene and dried in a vacuum oven to furnish 26.03 g and used as such (yield quantitative). MS: [M+H]+=197 (low current)

### Step 2: Synthesis of 4-phenyl-N-benzylsuccinimide

268 mg 4-Bromobenzylsuccinimide (1 mmol), 122 mg benzene boronic acid (1 mmol), 202 mg triethylamine (2 mmol) and 78.6 mg trans-dichlorobis(tri-o-tolylphosphine)-palladium (II) were added to 50 ml acetonitrile. After 3 nights refluxing, 60-65% conversion was obtained, and was followed by evaporation and recrystallisation from ethylacetate/hexane, which was continued by filtration, dissolving in methylene chloride, and purification on silicagel. After evaporation an oil with 60-65% purity (LCMS) was isolated. A 58.1% yield was reached. The product was used in the next reaction step as such.

### Step 3: Synthesis of 7-biphenyl-4-ylmethyl-5,9-dihydroxy-pyrrolo[3,4-g]quinoxaline-6,8-dione

642 mg 4-Phenylbenzyl-N-succinimide (2.43 mmol), 500 mg 2,3-pyrazinemethyl-dicarboxylate (2.55 mmol), 225 mg NaH 60% in parafine oil (5.63 mmol) and 6 drops methanol were added to 20 ml THF. Refluxing was applied till all reagents had reacted (TLC Ethylacetate/Hexane: 70/30). Excess NaH was neutralised with water, 100 ml in total, and was followed with the evaporation of THF. The aqueous solution was acidified with HCl 37% till pH=1.50 ml diethylether was added and the mixture was shaken vigorously. The precipitate was filtered off, rinsed with diethylether and dried in a vacuum oven. The precipitate had a purity of 97.9% by LCMS. A 18.8% yield was reached. MS: [M+H]⁺=398, [M−H]⁻=396

### EXAMPLE 5

### Preparation of 7-Benzo[1,3]dioxol-5-ylmethyl-5-hydroxy-9-(3-methyl-butoxy)-pyrrolo[3,4-g]quinoxaline-6,8-dione

A solution of 7-Benzo[1,3]dioxol-5-ylmethyl-5,9-dihydroxy-pyrrolo[3,4-g]quinoxaline-6,8-dione (365 mg, 1.0 mmol), 1-Bromo-3-methylbutane (151 mg, 1.0 mmole), potassium carbonate (138 mg, 1.0 mmole) in DMF was warmed at 100° C. overnight. This solution was evaporated to dry, washed with water and the organic phase extracted with ethyl acetate. After drying on magnesium sulfate, filtration, evaporation of solvent, purification, 10 mg were isolated and analysed through LC/MS. MS: [M+H]⁺=436, [M−H]⁻=434. ES−: 434, 263, 227. ES+: 436, 366, 314, 244, 135.

### EXAMPLE 6

Antiviral Analyses

The compounds of the present invention were examined for anti-viral activity in a cellular assay. The assay demonstrated that these compounds exhibited potent anti-HIV activity against a wild type laboratory HIV strain (HIV-1 strain LAI, named as “IIIB”) and a panel of mutant viruses with multi-drug resistance. The cellular assay was performed according to the following procedure:

HIV- or mock-infected MT4 cells equipped with a LTR-GFP reporter were incubated for three days in the presence of various concentrations of the inhibitor. At the end of the incubation period, the replicating virus in the control cultures had killed all HIV-infected cells in the absence of any inhibitor. The anti-viral replication assay is based on a GFP readout, and directly measures the ongoing replication of virus in MT4 cells via the specific interaction of HIV-tat with LTR-sequences coupled to GFP. The inhibitory activity of the compound was monitored on the virus-infected cells and was expressed as IC₅₀. This value represents the amount of the compound required to protect 50% of the cells from the cytopathogenic effect of the virus. The toxicity (Tox) of the compound was measured on the mock-infected cells and was expressed as CC₅₀, which represents the concentration of compound required to inhibit the growth of the cells by 50%. The toxicity assay is also based on GFP-readout, where a reduced expression of the GFP reporter protein serves as a marker for cellular toxicity of a compound. The selectivity index (SI) (ratio CC₅₀/IC₅₀) is an indication of the selectivity of the anti-HIV activity of the inhibitor.

Because of the increasing emergence of drug resistant HIV strains, the compounds were tested for their potency against different drug-resistant HIV-1 strains. Strains SM026, SM052, and T13299 are strains containing mutations that cause resistance against reverse transcriptase inhibitors. T13275 is a HIV-strain containing multi-drug (reverse transcriptase and protease) resistance mutations. SM026 (V003I, K103N, Y181C, E224D/E, P313P/S), SM052 (V003I, K101E, K103N), T13299 (V003I, L1100I, K103N, E138G, V179I, Y181C, L214F, V276V/I, A327A/V), T13275 (V003I, L010F, I013V, V032T, S037N, M046I, I047V, I050V, L063P, A071V, I084V, L089V, T091A, Q092R, K020R, E028K, M041L, K043E, E044A, D067N, L0741, K103N, V118I, D123N, S162C, Y181C, G196K, Q207E, L210W, R211K, L214F, T215Y, K219N, P225H, D250E, P272A, R277K, I293V, P294T, E297K, K311R, R358K, T376A, E399D, T400L).

Enzymatic Integrase Assay

The activity of HIV-integrase was determined using an oligonucleotide-based assay in which the DNA strand transfer by preformed complexes of integrase and processed DNA was measured by means of an enzyme-linked immunosorbent assay (ELISA) in microtiter plate format. Recombinant His-tagged HIV-1 integrase was produced in the E. coli strain BL21(DE3) from the plasmid pINSD.His.sol (available from NTH) after induction with isopropyl-β-D-thiogalactopyranoside (IPTG) according to described procedures (cfr. ref. Jenkins et al., “A soluble active mutant of HIV-1 integrase”, J. Biol. Chem. 271 (1196), 7712-7718). HPLC-purified oligodeoxynucleotides were obtained from Proligo, and used for preparation of the viral DNA substrate and target DNA.

| | | |
|---|---|---|
| INb-1C: | 5′ -bGTGTGGAAAATCTCTAGCAGT- 3′ | SEQ. ID. 1 |
| IN-1NC: | 5′ -ACTGCTAGAGATTTTCCACAC- 3′ | SEQ. ID. 2 |
| INT5: | 5′ -TGACCAAGGGCTAATTCACTf- 3′ | SEQ. ID. 3 |
| INT6: | 5′ -AGTGAATTAGCCCTTGGTCAf- 3′ | SEQ. ID. 4 |

INb-1C is 5′-biotinylated, INT5 and INT6 are at the 3′-end labeled with FITC. INb-1C and IN-1NC correspond to the U5 end of the HIV-1 LTR. The DNA substrate for the integrase reactions was made by annealing INb-1C and IN-1NC. An equimolar mixture of INb-1C and IN-1NC was heated shortly at 95° C. in the presence of 100 mM NaCl and allowed to cool slowly to room temperature. In the same way, INT5 and INT6 were annealed to produce a target DNA molecule.

The integration strand transfer reactions were performed in the following way:

20 nM biotinylated DNA substrate INb-1C/IN-1NC was pre-incubated with 300 nM HIV-integrase at 37° C. for 5 min, to allow the cleavage reaction to occur. The candidate compounds and 50 nM target DNA INT5/INT6 were added to the reaction mix containing 20 mM Hepes pH 7.5, 25 mM NaCl, 5 mM MnCl₂, 2 mM DTT, and 50 μg/ml BSA, and incubated for 2 h at 37° C. The reaction mix was transferred to streptavidin-coated plates (Exiqon), which were prewashed (three times) with 5×SSCT, and incubated for 1 h at room temperature to allow capture of the biotinylated viral DNA/target DNA complex. Plates were washed three times with 2×SSCT buffer, and anti-FITC POD-coupled antibody (Roche) was added and incubated for 1 h at room temperature to detect integrated FITC-labeled target DNA. After a final washing step with PBST (5 times), BM chemiluminescent POD-substrate (Roche) was added, and luminescence was read out.

The table below list the pIC₅₀ values for those compounds tested in the enzymatic integrase assay. The pIC₅₀ is expressed in Molar units and is the value according to the following formula:
pIC₅₀=−log IC₅₀

wherein the IC₅₀ value is the drug concentration at which 50% of the enzyme or viral activity is inhibited.

| Compound | pIC₅₀ |
|---|---|
| 1 | 6.21 |
| 3 | 5.8 |
| 2 | 6.38 |
| 4 | 6.66 |
| 21 | 6.06 |
| 22 | 6.40 |
| 18 | 5.12 |
| 19 | 5.38 |
| 20 | 5.67 |
| 8 | 4.97 |
| 26 | 5.66 |
| 17 | 6.25 |
| 6 | 5.50 |
| 24 | 5.60 |
| 27 | 5.85 |
| 14 | 5.69 |
| 16 | 6.25 |
| 7 | 5.31 |
| 30 | 4.62 |
| 23 | 5.83 |
| 15 | 5.98 |
| 31 | <4.00 |
| 37 | 5.83 |
| 35 | 5.74 |
| 5 | 5.25 |
| 25 | 5.69 |
| 12 | <4.00 |
| 42 | <4.00 |
| 11 | <4.00 |
| 13 | <4.00 |
| 38 | 6.32 |
| 40 | 5.95 |
| 39 | 5.69 |
| 29 | 5.17 |
| 34 | 5.06 |
| 10 | 5.00 |
| 36 | 4.74 |
| 33 | 4.66 |
| 28 | 4.18 |
| 32 | 4.98 |

Time of Addition Assay

In a time-of-addition experiment, the step in the HIV replication cycle in which a compound is active was determined and compared with reference compounds including inhibitors for binding/fusion, reverse transcriptase, integrase and protease. When a potent antiviral compound was added at the time of infection, no viral replication took place. But, if addition of compound was delayed, protection was observed up to the moment that the virus had passed the stage at which the inhibitor interacted. The use of reference compounds with a known mode of action was essential for the correct interpretation of the results.

MT4-LTR-EGFP cells were infected at a high multiplicity of infection (MOI) by centrifugation for 10 min at 1200 g. Unadsorbed virus was removed by two washing steps at 4° C. in order to synchronize the infection. From 30 min post infection on, the compounds 1-43 were added to parallel cultures in microtiter plates at different times. The cultures were scored microscopically for fluorescence 24 hours after infection and supernatant was collected. HIV replication in the supernatant samples was quantified by measuring the concentration of the p24 viral antigen using a commercial kit, according to the manufacturer protocol (NEN). Because of the high MOT used in this type of experiments, concentrations of inhibitors were at least 100 fold higher than their EC₅₀ value in the cellular antiviral assay. The score of the compounds 1-41 was integrase.

Analysis of HIV Integration Using Real-Time PCR

For confirmation that compounds inhibited the viral replication cycle at the integration step, DNA extracts of cells infected with HIV in the absence or presence of compounds were analysed by quantitative PCR. Next to the detection of integrated DNA (proviral DNA), the production of 2LTR-circles formed by circularisation of unintegrated DNA was monitored. Specific inhibition of the integration of viral DNA into the genome was typically associated with accumulation of 2LTR-circles in the nucleus. MT4 cells were infected with HIV at high MOI by centrifugation for 60 min at 1200 g. After infection cells were incubated in the presence of compound in 24-well plates (10⁶ c/well) for 16 h, and DNA was extracted using the QiaAmp DNA mini kit (Qiagen).

After normalization, 2LTR-circles and integrated DNA were quantified by real-time PCR using the appropriate primers and probe (cfr. reference Butler et al.). Reactions were analysed using the ABI Prism 5700 sequence detection system (Applied Biosystems).

DNA was analyzed for integration (Alu-PCR) and 2LTR-circles, 16 h after infection. DNA was also analyzed for viral cDNA synthesis, 4 h after infection. Results are shown in Table 2.

TABLE 2

| | Integration | | |
|---|---|---|---|
| Compound | Alu-PCR | 2LTR-circles | Conclusion |
| Control | + | + | |
| Reference | − | +++ | Integrase |
| compound | | | |
| NNRTI | − | − | RT |
| Compound 1 | − | +++ | Integrase |
| Compound 2 | − | +++ | Integrase |

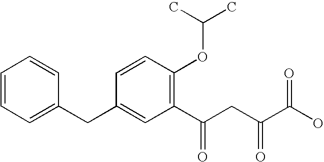

Reference compound with known integrase inhibitory activity
In Table 2, symbol “+” indicates the amount of DNA copies in the absence of a compound, that is in the control reaction, for both integrated DNA and 2LTR-circles:
“++” symbol indicates a 2-to-5 fold increase in the amount of DNA copies in comparison with the control level
“+++” symbol indicates an increase in the amount of DNA copies > 5 in comparison with the control level
“−” symbol indicates an amount of DNA copies near or below the detection limit.

### EXAMPLE 7

### Preparation of toluene-4-sulfonic acid 7-benzo[1,3]dioxol-5-ylmethyl-9-hydroxy-6,8-dioxo-7,8-dihydro-6H-pyrrolo[3,4-g]quinoxalin-5-yl ester

After adding 221 μl Et₃N (1.57 mmol, d=0.72) to a suspention of 7-benzo[1,3]dioxol-5-ylmethyl-5,9-dihydroxy-pyrrolo[3,4-g]quinoxaline-6,8-dione (501 mg, 1.37 mmol) in 30 ml CH₂Cl₂, the RM turns from yellow to dark red and becomes clear. A solution of TsCl (262 mg, 1.40 mmol) in 10 ml CH₂Cl₂ was added drop wise and the RM was stirred for 3 days at RT. To the RM was added H₂O and Et₃N and extracted. The organic layer was dried with Na₂SO₄, filtered and CH₂Cl₂ was evaporated. The desired product was purified by chromatography (SiO₂, CH₂Cl₂/MeOH: gradient 0% to 10% MeOH) (8.7 mg, 1.2% yield, 90% pure). MS: [M+H]⁺=520; [M−H]⁻=518.

### EXAMPLE 8

### Preparation of 7-benzo[1,3]dioxol-5-ylmethyl-9-hydroxy-5-methyl-pyrrolo[3,4-g]quinoline-6,8-dione

### Step 1: Preparation of 3-(2-methyl-[1,3]dioxolan-2-yl)-pyridine-2-carboxylic acid isopropyl ester

A mixture of isopropyl 3-acetyl-pyridine-2-carboxylic acid isopropyl ester (6.00 g, 0.029 mol), p-toluenesulfonic acid monohydrate (5.80 g, 0.030 mol) and ethylene glycol (2.70 g, 0.043 mol) in 100 ml toluene was refluxed for 17 hours with a Dean-Stark apparatus. After cooling, the RM was basified with solid Na₂CO₃. After filtration and evaporation, the residue was purified by chromatography (SiO₂, CH₂Cl₂/MeOH: gradient 0% to 10% MeOH) to obtain a yellow oil (4.21 g, 58% yield, 87% pure). MS: [M+H]⁺=252.

### Step 2: Preparation of 1-benzo[1,3]dioxol-5-ylmethyl-3-{hydroxy-[3-(2-methyl-[1,3]dioxolan-2yl)pyridin-2yl]-methylene}-pyrrolidine-2,5-dione

3-(2-methyl-[1,3]dioxolan-2-yl)-pyridine-2-carboxylic acid isopropyl ester (202 mg, 0.81 mmol) and 1-benzo[1,3]dioxol-5-ylmethyl-pyrrolidine-2,5-dione (178 mg, 0.77 mmol) were dissolved in THF (30 ml). After NaH 60% (1.35 mmol) and MeOH (3 drops) were carefully added, the suspension was heated at reflux for 6 days. After evaporation, the residue was dissolved in acidic water and ether. This heterogeneous mixture was then vigorously stirred for several hrs. The precipitate was then filtered off. The filtrate was evaporated and purified by chromatography (SiO₂, CH₂Cl₂/MeOH: gradient 5% to 10% MeOH) (170 mg, 52% yield, 62% pure). MS: [M+H]⁺=425; [M+Na]⁺=447; [M−H]⁻=423.

### Step 3: Preparation of 3-[(3-acetyl-pyridin-2-yl)-hydroxy-methylene]-1-benzo[1,3]dioxol-5-ylmethyl-pyrrolidine-2,5-dione

1-benzo[1,3]dioxol-5-ylmethyl-3-{hydroxy-[3-(2-methyl-[1,3]dioxolan-2yl)pyridin-2yl]-methylene}-pyrrolidine-2,5-dione (171 mg, 0.40 mmol) was mixed with 9 ml H₂O and 1 ml concentrated HCl and refluxed for 30 min. After evaporation, the residue was co-evaporated twice with toluene to obtain a yellow oil which was used as such in the next step (188 mg, 73% yield, 60% pure). MS: [M+H]⁺=381; [M−H]⁻=379.

### Step 4: Preparation of 7-benzo[1,3]dioxol-5-ylmethyl-9-hydroxy-5-methyl-pyrrolo[3,4-g]quinoline-6,8-dione

The residue of step 3 (188 mg, 60% pure, 0.30 mmol) was dissolved in 20 ml THF. After NaH 60% (3.28 mmol) and MeOH (4 drops) were carefully added, the suspension was heated at reflux for 16 hours and stirred at RT for 24 hours. When some HOAc was added, the RM was evaporated and co-evaporated two times with toluene. The desired product was purified by preparative HPLC (Waters Xterra Prep MS C₁₈ (5 μm, 19×50 mm) eluting with 5-95% acetonitile/water (2% TFA) at 20 ml/min) (10 mg, 6% yield, 79% pure). MS: [M+H]⁺=363; [M−H]⁻=361.

### EXAMPLE 9

### Preparation of 2-[2-(4-fluoro-phenyl)-ethyl]-4,9-dihydroxy-2,5,6-triaza-cyclopenta[b]naphthalene-1,3-dione

Pyridazine-3,4-dicarboxylic acid diethyl ester (prepared according to reference J. Heterocyclic Chem., 27, 1990, 579-582) (494 mg, 2.20 mmol) and 1-[2-(4-Fluoro-phenyl)-ethyl]-pyrrolidine-2,5-dione (452 mg, 2.04 mmol) was dissolved in THF (100 ml). After NaH 60% (5.61 mmol) and MeOH (3 drops) were carefully added, the suspension was heated at reflux for 2 days. After evaporation, the residue was dissolved in acidic water and ether. This heterogeneous mixture was then vigorously stirred for several hours. The brown precipitate was filtered off, washed with some ether and dried in vacuo to get the product (117 mg, 15% yield, >95% pure). MS: [M+H]⁺=354; [M−H]⁻=352.

NMR: 9.59 (d, J=5.81, 1H); 8.46 (d, J=5.81, 1H); 7.28-7.19 (m, 2H); 7.13-7.04 (m, 2H); 3.80 (t, J=6.82, 2H); 2.93 (t, J=6.82, 2H).

### EXAMPLE 10

### Preparation of 7-benzo[1,3]dioxol-5-ylmethyl-5-hydroxy-pyrrolo[3,4-g]quinoxaline-6,8-dione

### Step 1: Preparation of trifluoro-methanesulfonic acid 7-benzo[1,3]dioxol-5-ylmethyl-9-hydroxy-6,8-dioxo-7,8-dihydro-6H-pyrrolo[3,4-g]quinoxalin-5-yl ester

To a mixture of 7-benzo[1,3]dioxol-5-ylmethyl-5,9-dihydroxy-pyrrolo[3,4-g]quinoxaline-6,8-dione (994 mg, 2.72 mmol) and 461 μl Et3N (d=0.72, 3.29 mmol) in 80 ml CH₂Cl₂ was added drop wise 407 μl trifluoromethanesulfonic anhydride (d=1.71, 2.47 mmol) which was diluted in 20 ml CH₂Cl₂. This RM was then stirred for 20 hours at RT. To the RM was added H₂O and Et₃N. After separation of the two layers, the organic layer was dried with MgSO₄, filtered and evaporated to dryness to obtain a brown crude oil. The product was used as such in the next step (850 mg, 62% yield, 65% pure). MS: [M+H]⁺=498.

### Step 2: Preparation of 7-benzo[1,3]dioxol-5-ylmethyl-5-hydroxy-pyrrolo[3,4-g]quinoxaline-6,8-dione

A mixture of the product obtained in step 1 (400 mg, 0.48 mmol, 65% pure), Pd/C 10% (0.5 g) and 5 ml Et₃N (d=0.72, 3.56 mmol) dissolved in 150 ml MeOH, and was hydrogenated at atmospheric pressure during 2 hours. After filtration and evaporating the residue was purified by preparative HPLC (Waters Xterra Prep MS C₁₈ (5 μm, 19×50 mm) eluting with 5-95% acetonitrile/water (2% TFA) at 20 ml/min) (23.5 mg, 8% yield, 75% pure). MS: [M−H]⁻=348.

### EXAMPLE 11

### Preparation of 7-(3-bromo-benzyl)-5,9-dihydroxy-2-methyl-pyrrolo[3,4-g]quinoxaline-6,8-dione

### Step 1: Preparation of 5-methyl-pyrazine-2,3-dicarboxylic acid dimethyl ester

5-methyl-pyrazine-2,3-dicarboxylic acid (prepared according to reference Chem. Ber., 114, 1981, 240-245) (10.6 g, 33% pure, 19.2 mmol) was dissolved in MeOH and the pH was adjusted to 2 with HCl (a solution of 7N in i-PrOH). This mixture was heated at reflux for 24 hrs. After evaporating MeOH, the residue was dissolved in CH₂Cl₂ and washed twice with NaHCO₃. The organic layer was dried with MgSO₄, filtered, evaporated to dryness and dried in a vacuum oven to get a crude red oil, witch was used in the next step without further purification (1.3 g, 11% yield, 63% pure). MS: [M+H]⁺=211.

### Step 2: Preparation of 7-(3-bromo-benzyl)-5,9-dihydroxy-2-methyl-pyrrolo[3,4-g]quinoxaline-6,8-dione

5-methyl-pyrazine-2,3-dicarboxylic acid dimethyl ester (112 mg, 63% pure, 0.36 mmol), obtained in step 1 and 1-(3-bromo-benzyl)-pyrrolidine-2,5-dione (112 mg, 0.42 mmol) were dissolved in THF (20 ml). After NaH 60% (3.10 mmol) and MeOH (5 drops) were carefully added, the suspension was heated at reflux for 1.5 hours. After evaporation, the residue was dissolved in acidic water and ether. This heterogeneous mixture was then vigorously stirred for several hrs. The brown precipitate was then filtered, washed with some ether and dried in vacuo to obtain the desired product (59 mg, 34%, yield, 90% pure). MS: [M+H]⁺=414; [M−H]⁻=412.

### EXAMPLE 12

### Preparation of 7-(3,4-dichloro-benzyl)-5,9-dihydroxy-2-methyl-pyrrolo[3,4-g]quinoxaline-6,8-dione

5-methyl-pyrazine-2,3-dicarboxylic acid dimethyl ester (126 mg, 63% pure, 0.38 mmol) and 1-(3,4-dichloro-benzyl)-pyrrolidine-2,5-dione (138 mg, 0.53 mmol) were dissolved in THF (10 ml). After NaH 60% (2.42 mmol) and MeOH (5 drops) were carefully added, the suspension was heated at reflux for 2 hours. After evaporation, the residue was dissolved in acidic water and ether. This heterogeneous mixture was then vigorously stirred for several hrs. The brown precipitate was then filtered, washed with some ether and dried in vacuo to obtain the desired product (30 mg, 12% yield, 95% pure). MS: [M+H]⁺=404; [M−H]⁻=402.

NMR: 11.31-11.11 (br. s, 1H); 10.96-10.79 (br. s, 1H); 9.01 (s, 1H); 7.60 (d, J=8.08, 1H); 7.59 (d, J=2.53, 1H); 7.31 (dd, J=8.08, J=1.77, 1H); 4.74 (s, 2H); 2.82 (s, 3H).

### EXAMPLE 13

Preparation of 7-(3-brome-benzyl)-5,9-dihydroxy-2,3-dimethyl-pyrrolo[3,4-g]quinoxaline-6,8-dione

### Step 1: Preparation of 5,6-dimethyl-pyrazine-2,3-dicarboxylic acid dimethyl ester

5,6-dimethyl-pyrazine-2,3-dicarboxylic acid (prepared according to reference Chem. Ber., 114, 1981, 240-245) (19.6 g, 61% pure, 61 mmol) was dissolved in MeOH and the pH was adjusted to 2 with HCl (a solution of 7N in i-PrOH). This mixture was heated at reflux for 2 days. After evaporation, the residue was dissolved in CH₂Cl₂ and washed twice with NaHCO₃ solution. The organic layer was dried with MgSO₄, filtered, evaporated to dryness and dried in a vacuum oven to get a crude orange solid, witch was used in the next step without further purification (1.40 g, 10% yield, 55% pure). MS: [M+H]⁺=225

### Step 2: Preparation of 7-(3-brome-benzyl)-5,9-dihydroxy-2,3-dimethyl-pyrrolo[3,4-g]quinoxaline-6,8-dione

The crude product of step 1 (117 mg, 55% pure, 0.29 mmol) and 1-(3-bromo-benzyl)-pyrrolidine-2,5-dione (105 mg, 0.39 mmol) were dissolved in THF (10 ml). After NaH 60% (1.51 mmol) and MeOH (5 drops) were carefully added, the suspension was heated at reflux for 18 hours. When THF was evaporated, the residue was dissolved in acidic water and ether. This heterogeneous mixture was then vigorously stirred for several hours. The precipitate was then filtered off, washed with some ether and dried in a vacuum oven to obtain the desired product (83.4 mg, 50% yield, >95% pure). MS: [M+H]⁺=430; [M−H]⁻=428.

NMR: 7.55-7.51 (br. s.; 1H), 7.51-7.46 (m; 1H), 7.35-7.28 (m; 2H), 4.73 (s; 2H), 2.77 (s, 6H).

### EXAMPLE 14

### Preparation of 7-(3-brome-benzyl)-2-ethoxy-5,9-dihydroxy-pyrrolo[3,4-g]quinazoline-6,8-dione

### Step 1 Preparation of 2-methylsulfanul-pyrimidine-4,5-dicarboxylic acid diethyl ester

Ethyl 4-chloro-2-methylthiopyrimidine-5-carboxylate (2.0 g, 0.0086 mol), 1,1′-bisdiphenylphosphinoferrocene palladium (0.35 g) and sodium acetate (1.4 g, 0.017 mol) were dissolved in 150 ml EtOH in a Parr reactor. The RM was subjected to 25 bar CO and heated at 140° C. overnight. The RM was filtered through dicalite and evaporated. The residue was dissolved in CH₂Cl₂. To this solution was added some silica and vigorously stirred for several hrs. The mixture was filtered and evaporated to dryness to get the desired crude product, which was used as such in the next step (5.14 g, 88% yield, 92% pure). MS: [M+H]⁺=271.

### Step 2: Preparation of 2-ethoxy-pyrimidine-4,5-dicarboxylic acid diethyl ester

To a solution of the crude product of step 1 (100 mg, 0.37 mmol) in 5 ml EtOH was added 281 μl sodium-ethanolate (d=0.87, 0.79 mmol) and the RM was refluxed for 9 days. After the solvent was evaporated, this residue was used as such in the next step (100 mg, 43% yield, 43% pure). MS: [M+H]⁺=269; [M−H]⁻=267.

### Step 3: Preparation of 7-(3-brome-benzyl)-2-ethoxy-5,9-dihydroxy-pyrrolo[3,4-g]quinazoline-6,8-dione

The ester made in step 2 (100 mg, 43% pure, 0.16 mmol) and 1-(3-bromo-benzyl)-pyrrolidine-2,5-dione (91 mg, 0.34 mmol) were dissolved in THF (20 ml). After NaH (1.43 mmol) and EtOH (5 drops) were carefully added, the suspension was heated at reflux for 17 hours. After evaporation, the residue was dissolved in acidic water and ether. This heterogeneous mixture was then vigorously stirred for several hrs. The precipitate was then filtered, washed with some ether to get a crude product (11 mg, 12% yield, 75% pure). MS: [M+H]⁺=446; [M−H]⁻=444.

### EXAMPLE 15

### Preparation of 7-(3-brome-benzyl-5,9-dihydroxy-2-methoxy-pyrrolo[3,4-g]quinazoline-6,8-dione

To a solution of 2-methylthio-pyrimidine-4,5-dicarboxylic acid diethyl ester (500 mg, 1.85 mmol) and 1-(3-bromo-benzyl)-pyrrolidine-2,5-dione (472 mg, 1.76 mmol) were dissolved in THF (50 ml). After NaH (6.23 mmol) and MeOH (14 drops) were carefully added, the suspension was heated at reflux for 3 days. When THF was evaporated, the residue was dissolved in acidic water and ether. This heterogeneous mixture was then vigorously stirred for several hrs. The precipitate was then filtered, washed with some ether. The precipitate was purified by preparative HPLC (Waters Xterra Prep MS C₁₈ (5 μm, 19×50 mm) eluting with 5-95% acetonitrile/water (2% TFA) at 20 ml/min) to obtain the separated side product (10 mg, 1% yield, >95% pure). MS: [M+H]⁺=432; [M−H]⁻=430.

### EXAMPLE 16

### Preparation of Cyclopropanecarboxylic acid 7-(3,4-dichloro-benzyl)-9-cyclopropanecarbonyloxy-6,8-dioxo-7,8-dihydro-6H-pyrrolo[3,4-g]quinoxalin-5-yl ester

200 mg (0.513 mmol) 7-(3,4-Dichloro-benzyl)-5,9-dihydroxy-pyrrolo[3,4-g]quinoxaline-6,8-dione was suspended in 30 ml CH2Cl2 and 1.28 mmol Et3N was added followed by addition of 1.128 mmol Cyclopropanecarbonyl chloride. The RM was stirred 4 hours, concentrated to a small volume and quickly purified over a short column silica applying a vacuum and CH2Cl2/CH3OH 99/1 as eluent. The pure fractions were collected, concentrated and dried yielding 111 mg (41.1%) MS: [M+H]⁺=526.

NMR: ¹H (CDCl₃): δ 9.15 (s, 2H), 7.67 (d, J=2.00 Hz, 1H), 7.54 (d, J=8.26 Hz, 1H) 7.42 (dd, 1H, J=2.04 Hz and J=14.68 Hz, 1H), 4.91 (s, 2H), 2.30-2.22 (m, 2H), 1.57-1.49 (m, 4H), 1.39-1.29 (m, 4H).

### EXAMPLE 17

### Preparation of dodecanoic acid 7-benzo[1,3]dioxol-5-ylmethyl-9-hydroxy-6,8-dioxo-7,8-dihydro-6H-pyrrolo[3,4-g]quinoxalin-5-yl ester

7-Benzo[1,3]dioxol-5-ylmethyl-5,9-dihydroxy-pyrrolo[3,4-g]quinoxaline-6,8-dione (300 mg, 0.82 mmol), dodecanoic acid (331 mg, 1.65 mmol), 2-(1H-benzotriazol-1-yl)-1,1,3,3-tetramethyluronium tetrafluoroborate (TBTU) (580 mg, 1.81 mmol) and triethylamine (0.47 mL, 3.29 mmol) were dissolved in dimethylformamide (20 mL). The solution was stirred at room temperature for 24 hours. The solution was diluted with water (100 mL). The water layer was extracted with ethyl acetate (3×40 mL). The organic layer was washed with brine (40 mL), dried over magnesium sulphate and concentrated under reduced pressure. The desired product was crystallised in dimethylformamide. The crystals were filtered off and dried in vacuo for 24 hours (99.10 mg, 22%, purity (LC)>95%). MS: [M+]⁺=548, [M−H]⁻=546.

NMR: ¹H (CDCl₃): δ 9.07 (d, J=1.79 Hz, 1H), 8.98 (d, J=1.76 Hz, 1H), 7.08-7.00 (m, 1H), 7.00-6.98 (m, 1H), 6.80 (d, J=7.82 Hz, 1H), 5.97 (s, 2H), 4.79 (s, 2H), 2.89 (t, J=7.61 Hz, 2H), 1.99-1.85 (m, 2H), 1.77-1.59 (brs, 1H), 1.59-1.49 (m, 4H), 1.49-1.24 (m, 12H), 0.92 (t, J=6.85 Hz, 3H).

### EXAMPLE 18

### Preparation of dodecanoic acid 7-(3,4-dichloro-benzyl)-9-hydroxy-6,8-dioxo-7,8-dihydro-6H-pyrrolo[3,4-g]quinoxalin-5-yl ester

7-(3,4-Dichloro-benzyl)-5,9-dihydroxy-pyrrolo[3,4-g]quinoxaline-6,8-dione (300 mg, 0.77 mmol), dodecanoic acid (310 mg, 1.54 mmol), TBTU (543 mg, 1.69 mmol) and triethylamine (0.44 mL, 3.08 mmol) were dissolved in dimethylformamide (20 mL). The solution was stirred at room temperature for 96 hours. The solution was diluted with water (150 mL) and extracted with ethyl acetate (5×50 mL). The organic layer was washed with brine (50 mL), dried over sodium sulphate and evaporated under reduced pressure. The residue was suspended in water. The brown crystals were filtered off and dried in vacuo for 24 hours (341 mg, 78%, purity (LC)>94%). MS: [M+H]⁺=572

### EXAMPLE 19

### Preparation of hexanoic acid 7-(3,4-dichloro-benzyl)-9-hydroxy-6,8-dioxo-7,8-dihydro-6H-pyrrolo[3,4-g]quinoxalin-5-yl ester

7-(3,4-Dichloro-benzyl)-5,9-dihydroxy-pyrrolo[3,4-g]quinoxaline-6,8-dione (300 mg, 0.77 mmol), hexanoic acid (0.20 mL, 1.54 mmol), TBTU (543 mg, 1.69 mmol) and triethylamine (0.44 mL, 3.08 mmol) were dissolved in dimethylformamide (20 mL). The solution was stirred at room temperature for 48 hours. The solution was diluted with water (200 mL). The aqueous layer was extracted with ethyl acetate (4×50 mL). The combined organic layers were washed with brine (50 mL) and dried over sodium sulphate. The organic layer was concentrated under reduced pressure to yield a yellow colored oil. The residue was suspended in cold ether and stirred for 20 minutes. The crystals were filtered off and washed with cold ether (220 mg, 58%, purity (LC)>91%). MS: [M+H]⁺=488

### EXAMPLE 20

### Preparation of hexanoic acid 7-benzo[1,3]dioxol-5-ylmethyl-9-hydroxy-6,8-dioxo-7,8-dihydro-6H-pyrrolo[3,4-g]quinoxalin-5-yl ester

7-Benzo[1,3]dioxol-5-ylmethyl-5,9-dihydroxy-pyrrolo[3,4-g]quinoxaline-6,8-dione (300 mg, 0.82 mmol), hexanoic acid (193 mg, 1.64 mmol), TBTU (580 mg, 1.81 mmol) and triethylamine (0.47 mL, 3.29 mmol) were dissolved in dimethylformamide (10 mL). The solution was stirred at room temperature for 96 hours. The solution was diluted with water (200 mL). The water layer was extracted with ethyl acetate (50 mL). The aqueous layer was acidified to pH 4 with 0.1N HCl solution. The water layer was extracted with ethyl acetate (5×50 mL). The combined organic layers were washed with brine (50 mL), dried over sodium sulphate and evaporated under reduced pressure to afford a yellow colored oil. The residue was suspended in cold ether and stirred for 10 minutes. The crystals were filtered off and dried in vacuo for 1 hour (176 mg, 46%, purity (LC)>91%). MS: [M+H]⁺=464

NMR: ¹H (CDCl₃): δ 9.01 (s, 1H), 8.98 (s, 1H), 7.01-6.90 (m, 2H), 6.75 (d, J=7.81 Hz, 1H), 5.92 (s, 2H), 4.73 (s, 2H), 2.85 (t, J=7.61 Hz, 2H), 1.96-1.84 (m, 2H), 1.57-1.33 (m, 4H), 0.94 (t, J=7.15 Hz, 31).

### EXAMPLE 21

### Preparation of octadecanoic acid 7-(3,4-dichloro-benzyl)-9-hydroxy-6,8-dioxo-7,8-dihydro-6H-pyrrolo[3,4-g]quinoxalin-5-yl ester

7-(3,4-Dichloro-benzyl)-5,9-dihydroxy-pyrrolo[3,4-g]quinoxaline-6,8-dione (300 mg, 0.77 mmol), octadecanoic acid (442 mg, 1.54 mmol), TBTU (543 mg, 1.69 mmol) and triethylamine (0.44 mL, 3.08 mmol) were dissolved in dimethylformamide (15 mL). The solution was stirred at room temperature for 120 hours. The solution was diluted with water (200 mL), extracted with ethyl acetate (4×50 mL) and dried over sodium sulphate. The organic layer was evaporated under reduced pressure. The residue was stirred in ether for 15 minutes. The crystals were filtered off. The filtrate was concentrated and stirred in ether for 15 minutes. The crystals were filtered off. The filtrate was concentrated and purified by preparative HPLC (Waters Xterra Prep MC C18 (5 μm, 19×50 mm), eluens: acetonitrile (A), H₂O (B) and 2% TFA (C) from 90A/5B/5C to 95B/5C in 10 minutes) (35 mg, 7%, purity (LC)>95%). MS: [M+H]⁺=656, [M−H]⁻=654.

NMR: ¹H (CDCl₃): δ 9.08 (d, J=1.75 Hz, 1H), 8.96 (d, J=1.71 Hz, 1H), 7.58 (s, 1H), 7.40 (d, J=8.26 Hz, 1H), 7.30 (dd, J=198 Hz and J=8.26 Hz, 1H), 4.80 (s, 2H), 2.85 (t, J=7.58 Hz, 2H), 1.92-1.80 (m, 2H), 1.78-1.58 (brs, 1H), 1.58-1.43 (m, 4H), 1.43-1.01 (m, 24H), 1.01-0.71 (m, 3H).

### EXAMPLE 22

### Preparation of 2,2-dimethyl-propionic acid 7-(3,4-dichloro-benzyl)-9-hydroxy-6,8-dioxo-7,8-dihydro-6H-pyrrolo[3,4-g]quinoxalin-5-yl ester

7-(3,4-Dichloro-benzyl)-5,9-dihydroxy-pyrrolo[3,4-g]quinoxaline-6,8-dione (250 mg, 0.64 mmol), 2,2-dimethyl-propionic acid (132 mg, 1.28 mmol), TBTU (453 mg, 1.41 mmol) and triethylamine (0.36 mL, 2.56 mmol) were dissolved in dimethylformamide (15 mL). The solution was stiffed at room temperature for 48 hours. The solution was diluted with water (200 mL) and extracted with ethyl acetate (4×50 mL). The organic layer was washed with brine (50 mL), dried over sodium sulphate and evaporated under reduced pressure. The residue was stirred in cold ether for 15 minutes. The crystals were filtered off. The filtrate was concentrated and purified by preparative HPLC (Waters Xterra Prep MC C₁₈ (5 μm, 19×50 mm), eluens: acetonitrile (A), H₂O (B) and 2% TFA (C) from 90A/5B/5C to 95B/5C in 10 minutes) (30 mg, 10%, purity (LC)>93%). MS: [M+H]⁺=474.

### EXAMPLE 23

### Preparation of hexadecanoic acid 7-benzo[1,3]dioxol-5-ylmethyl-9-hexadecanoyloxy-6,8-dioxo-7,8-dihydro-6H-pyrrolo[3,4-g]quinoxalin-5-yl ester

7-Benzo[1,3]dioxol-5-ylmethyl-5,9-dihydroxy-pyrrolo[3,4-g]quinoxaline-6,8-dione (250 mg, 0.68 mmol) and triethylamine (0.21 mL, 1.51 mmol) were dissolved in dichloromethane (20 mL), followed by the dropwise addition of hexadecanoyl chloride (0.43 mL, 1.37 mmol). The solution was stirred at room temperature for 48 hours. The solution was partially evaporated under reduced pressure. The residue was purified by column chromatography (SiO₂) on elution with dichloromethane. A mixture of desired product and a certain amount of hexadecanoyl chloride was obtained. The residue was suspended in tetrahydrofurane (10 mL), followed by the addition of 3-(ethylenediamino)-propyl-functionalized silica gel (1.77 g, 2.40 mmol, 2.70 mmol N/g) to remove the amount of hexadecanoyl chloride. The suspension was stirred at room temperature for 1 hour. The resin was filtered off and washed with tetrahydrofurane. The filtrate was evaporated to yield white crystals. The crystals were recrystalized in chloroform/acetonitrile. The white crystals were filtered off and washed with acetonitrile and dried in vacuo for 1 hour (30 mg, 5%, purity (LC)>99%).

NMR: ¹H (CDCl₃): δ 9.04 (s, 2H), 7.01 (s, 1H), 6.98 (d, J=9.05 Hz, 1H), 6.83 (d, J=7.80 Hz, 1H), 6.00 (s, 2H), 4.81 (s, 2H), 2.92 (t, J=7.59 Hz, 4H), 2.00-1.87 (m, 4H), 1.68-1.50 (m, 4H), 1.50-1.24 (m, 44H), 0.91 (t, J=6.83 Hz, 6H).

¹³C (CDCl₃): δ 171.3 (CO), 163.9 (CO), 147.8, 147.4, 146.7, 142.2, 141.0, 129.3, 122.6, 121.1, 109.5, 108.4 (Cₐᵣₒₘ), 101.1 (CH₂), 42.0 (CH₂), 33.9, 31.9, 29.7, 29.7, 29.6, 29.5, 29.4, 29.3, 29.1, 24.7, 22.7 (CH₂), 14.1 (CH₃).

### EXAMPLE 24

### Preparation of 3-ethoxy-propionic acid 7-benzo[1,3]dioxol-5-ylmethyl-9-hydroxy-6,8-dioxo-7,8-dihydro-6H-pyrrolo[3,4-g]quinoxalin-5-yl ester

7-Benzo[1,3]dioxol-5-ylmethyl-5,9-dihydroxy-pyrrolo[3,4-g]quinoxaline-6,8-dione (300 mg, 0.82 mmol), 3-ethoxy-propionic acid (198 mg, 1.64 mmol), TBTU (580 mg, 1.81 mmol) and triethylamine (0.34 mL, 3.29 mmol) were dissolved in dimethylformamide (15 mL). The solution was stirred at room temperature for 24 hours. The solution was diluted with water (200 mL) and extracted with ethyl acetate (4×50 mL). The combined organic layers were dried over sodium sulphate and evaporated under reduced pressure. The residue was stirred in cold ether. The crystals were filtered off, washed with cold ether and dried in vacuo for 1 hour. The crystals were purified by preparative HPLC (Waters Xterra Prep MC C18 (5 μm, 19×50 mm), eluens: acetonitrile (A), H₂O (B) and 2% TFA (C) from 90A/5B/5C to 95B/5C in 10 minutes) (220 mg, 58%, purity (LC)>94%). MS: [M+M]⁺=466, [M−H]⁻=464.

NMR: ¹H (CDCl₃): δ 9.11 (d, J=1.69 Hz, 1H), 9.01 (d, J=1.54 Hz, 1H), 6.96 (s, 1H), 6.93 (d, J=8.72 Hz, 1H), 6.74 (d, J=7.78 Hz, 1H), 5.90 (s, 2H), 4.75 (s, 2H), 3.91 (t, J=6.73 Hz, 2H), 3.60 (q, J=3.60 Hz, 2H), 3.12 (t, J=6.73 Hz, 2H), 1.22 (t, J=7.00 Hz, 3H).

¹³C (CDCl₃): δ 168.5 (CO), 146.8, 146.2, 143.9, 121.6, 108.7, 108.5, 107.4 (Cₐᵣₒₘ), 100.1 (CH₂), 65.5 (CH₂), 64.4 (CH₂), 40.8 (CH2), 33.8 (CH₂), 14.1 (CH₃).

### EXAMPLE 25

### Preparation of 3-ethoxy-propionic acid 7-(3,4-dichloro-benzyl)-9-hydroxy-6,8-dioxo-7,8-dihydro-6H-pyrrolo[3,4-g]quinoxalin-5-yl ester

7-(3,4-Dichloro-benzyl)-5,9-dihydroxy-pyrrolo[3,4-g]quinoxaline-6,8-dione (157 mg, 0.40 mmol), 3-ethoxy-propionic acid (97 mg, 0.80 mmol), TBTU (284 mg, 0.88 mmol) and triethylamine (0.23 mL, 1.61 mmol) were dissolved in dimethylformamide (15 mL). The solution was stirred at room temperature for 24 hours. The solution was diluted with water (200 mL) and extracted with ethyl acetate (5×50 mL). The combined organic layers were dried over sodium sulphate and evaporated under reduced pressure. The crude product was purified by preparative HPLC (Waters Xterra Prep MC C18 (5 μm, 19×50 mm), eluens: acetonitrile (A), H₂O (B) and 2% TFA (C) from 90A/5B/5C to 95B/5C in 10 minutes) (6 mg, 3%, purity (LC)=88.11%). MS: [M+H]⁺=490.

### EXAMPLE 26

### Preparation of 7-(1-benzenesulfonyl-piperidin-3-ylmethyl)-5,9-dihydroxy-pyrrolo[3,4-g]quinoxaline-6,8-dione

Pyrazine-2,3-dicarboxylic acid dimethyl ester (173 mg, 0.88 mmol), 1-(1-benzenesulfonyl-piperidin-3-ylmethyl)-pyrrolidine-2,5-dione (269 mg, 0.80 mmol) and sodium hydride were dissolved in tetrahydrofurane (10 mL), followed by the addition of 5 drops of methanol. The solution was heated to reflux for 24 hours. The solution was concentrated. The residue was dissolved in water. The water solution was acidified to pH 4 with 1N HCl solution, followed by the addition of ether. The crystals were filtered off and dried in vacuo for 1 hour (245 mg, 65%, purity (LC)=95.68%). MS: [M+H]⁺=469.

### Preparation of 1-(1-benzenesulfonyl-piperidin-3-ylmethyl)-pyrolidine-2,5-dione

(1-Benzenesulfonyl-piperidin-3-ylmethyl)-carbamic acid tert-butyl ester (644 mg, 1.82 mmol), dihydro-furan-2,5-dione (184 mg, 1.82 mmol) and a catalytic amount of DMAP were dissolved in acetic acid (5 mL). The solution was heated to reflux for 4 days. The solvent was removed under reduced pressure. The residue was dissolved in dichloromethane (50 mL). The organic layer was washed with sodium bicarbonate (2×40 mL), dried over sodium sulphate and evaporated under reduced pressure. The residue was dissolved in hot methanol. After cooling, white crystals were precipitated and filtered off (269 mg, 44%, purity (LC)=94.56%). MS: [M+H]⁺=337.

### Preparation of (1-benzenesulfonyl-piperidin-3-ylmethyl)-carbamic acid tert-butyl ester

Piperidin-3-ylmethyl-carbamic acid tert-butyl ester (1.00 g, 4.67 mmol) and triethylamine (1.99 mL, 14.00 mmol) were dissolved in dichloromethane (10 mL). Benzenesulfonyl chloride (0.60 mL, 4.67 mmol) was added dropwise. The solution was stirred at room temperature for 5 days. The solution was diluted with dichloromethane (40 mL), washed with 10% citric acid solution (2×40 mL). The organic layer was dried over sodium sulphate and evaporated under reduced pressure. MS: [M+H]⁺=355.

NMR: ¹H (CDCl₃): δ 7.72 (dd, J=1.36 Hz and J=19.90 Hz, 2H), 7.60-7.55 (m, 1H), 7.55-7.50 (m, 2H), 4.91-4.81 (m, 1H), 3.62-3.47 (m, 2H), 3.12-3.01 (m, 1H), 3.01-2.90 (m, 1H), 2.43-2.31 (m, 1H), 2.20-2.10 (in, 1H), 1.88-1.78 (m, 1H), 1.78-1.60 (m, 2H), 1.60-1.50 (m, 1H), 1.41 (s, 9H), 1.01-0.89 (m, 1H).

### EXAMPLE 27

Capsules

Preparation of Capsules

Active ingredient, in casu a compound of formula (I), is dissolved in organic solvent such as ethanol, methanol or methylene chloride, preferably, a mixture of ethanol and methylene chloride. Polymers such as polyvinylpyrrolidone copolymer with vinyl acetate (PVP-VA) or hydroxypropylmethylcellulose (HPMC), typically 5 mPa·s, are dissolved in organic solvents such as ethanol, methanol methylene chloride. Suitably the polymer is dissolved in ethanol. The polymer and compound solutions are mixed and subsequently spray dried. The ratio of compound/polymer was selected from 1/1 to 1/6. Intermediate ranges are 1/1.5 and 1/3. A suitable ratio is 1/6. The spray-dried powder, a solid dispersion, is subsequently filled in capsules for administration. The drug load in one capsule ranges between 50 and 100 mg depending on the capsule size used.

### EXAMPLE 28

Film-Coated Tablets

Preparation of Tablet Core

A mixture of 100 g of active ingredient, in casu a compound of formula (I), 570 g lactose and 200 g starch is mixed well and thereafter humidified with a solution of 5 g sodium dodecyl sulfate and 10 g polyvinylpyrrolidone in about 200 ml of water. The wet powder mixture is sieved, dried and sieved again. Then there was added 100 g microcrystalline cellulose and 15 g hydrogenated vegetable oil. The whole is mixed well and compressed into tablets, giving 10.000 tablets, each comprising 10 mg of the active ingredient.

Coating Process

To a solution of 10 g methylcellulose in 75 ml of denaturated ethanol there is added a solution of 5 g of ethylcellulose in 150 ml of dichloromethane. Then there are added 75 ml of dichloromethane and 2.5 ml 1,2,3-propanetriol. 10 g of polyethylene glycol is molten and dissolved in 75 ml of dichloromethane. The latter solution is added to the former and then there are added 2.5 g of magnesium octadecanoate, 5 g of polyvinylpyrrolidone and 30 ml of concentrated color suspension and the whole is homogenated. The tablet cores are coated with the thus obtained mixture in a coating apparatus.

1. A compound having the formula (I), 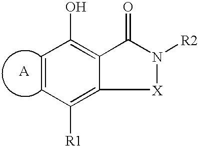
and their N-oxides, salts, stereoisomeric forms, racemic mixtures, prodrugs, esters and metabolites thereof, wherein
X is 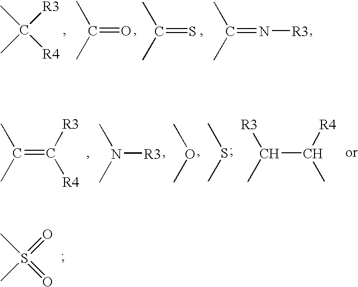
A, also mentioned as “A-ring”, together with the two carbons of the phenyl ring to which it is attached forms a monocyclic aryl or a monocyclic Het²;
R¹ is hydrogen, halogen, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R² is hydrogen, C₃₋₇cycloalkyl, aryl, Het¹, Het², C(═O)—R⁵, S(═O)_{y}—R⁶, OR⁷, NR⁸R⁹, C(═NR⁸)—R⁵, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R³ is hydrogen, halogen, nitro, cyano, C₃₋₇cycloalkyl, aryl, C(═O)—R⁵, S(═O)_{y}—R⁶, OR⁷, NR⁸R⁹, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, C(═O)—R⁵, OR⁷, and NR⁸R⁹;
R⁴ is hydrogen, halogen, nitro, cyano, C₃₋₇cycloalkyl or C₁₋₆alkyl;
y represents an integer being zero, one or two;
R⁵ is hydrogen, C₃₋₇cycloalkyl, aryl, Het¹, Het², C(═O)—R¹⁰, OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁶ is hydrogen, aryl, C₃₋₇cycloalkyl, Het¹, Het², OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁷ is hydrogen, aryl, C₃₋₇cycloalkyl, Het¹, Het², C(═O)—R¹⁰, S(═O)_{y}—R¹¹, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁸ is hydrogen, aryl, Het¹, Het², C₁₋₆alkyl, C₂₋₆alkenyl, C₂₋₆alkynyl, C₃₋₇cycloalkyl or polyhaloC₁₋₆alkyl;
R⁹ is hydrogen, aryl, C₃₋₇cycloalkyl, Het¹, Het², C(═O)—R¹⁰, S(═O)_{y}—R¹¹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹² and NR⁸R¹³;
R¹⁰ is hydrogen, C₃₋₇cycloalkyl, aryl, Het¹, Het², C(═O)—R⁸, C(═O)—OR⁸, C(═O)—NR⁸R⁸, OR⁸, O—C(═O)—R⁸, O—S(═O)_{y}—R⁸, S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹¹ is hydrogen, C₃₋₇cycloalkyl, aryl, Het¹, Het², OR⁸, O—C(═O)—R⁸, O—S(═O)_{y}—R⁸, NR⁸R⁸, NR⁸—C(═O)—R⁸; NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹² is hydrogen, C₃₋₇cycloalkyl, aryl, Het¹, Het², C(═O)—R⁸, C(═O)—OR⁸, C(═O)_{y}—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹³ is hydrogen, C₃₋₇cycloalkyl, aryl, Het¹, Het², C(═O)—R⁸, C(═O)—OR⁸, C(═O)—NR⁸R⁸, S(═O)—R⁸, S(═O)_{y}—R⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹⁴ is hydrogen, phenyl, C₁₋₆alkyl, C₂₋₆alkenyl, C₂₋₆alkynyl, C₃₋₇cycloalkyl;
aryl as a group or part of a group represents a monocyclic or polycyclic aromatic or a partially saturated monocyclic or polycyclic carbocycles wherein any such carbocycle within the meaning of aryl may have up to 14 carbon atoms and may be optionally substituted with one or more substituents independently selected from halogen, nitro, oxo, cyano, C₃₋₇cycloalkyl, Het¹, Het², C(═O)—R⁸, S(═O)_{y}—R¹⁴, OR¹⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het¹ as a group or part of a group represents a saturated or partially unsaturated monocyclic, bicyclic or tricyclic heterocycle having 3 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het² as a group or part of a group represents an aromatic monocyclic, bicyclic or tricyclic heterocycle having 5 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
for use as a medicine.

2. (canceled)

3. A compound having the formula (I) 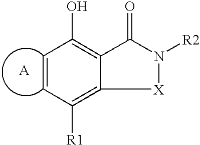
its N-oxide, salt, stereoisomeric form, racemic mixture, prodrug, ester or metabolite thereof, wherein
X is 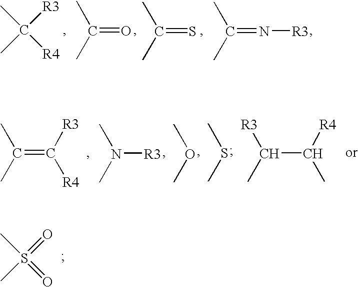
A, also mentioned as “A-ring”, together with the two carbons of the phenyl ring to which it is attached forms a monocyclic aryl or a monocyclic Het²;
R¹ is hydrogen, halogen, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶ OR⁷, and NR⁸R⁹;
R² is hydrogen, C₃₋₇cycloalkyl, aryl, Het¹, Het², C(═O)—R⁵, S(═O)_{y}—R⁶, OR⁷, NR⁸R⁹, C(═NR⁸)—R⁵, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R³ is hydrogen, halogen, nitro, cyano, C₃₋₇cycloalkyl, aryl, C(═O)—R⁵, S(═O)_{y}—R⁶, OR⁷, NR⁸R⁹, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, C(═O)—R⁵, OR⁷, and NR⁸R⁹;
R⁴ is hydrogen, halogen, nitro, cyano, C₃₋₇cycloalkyl or C₁₋₆alkyl;
y represents an integer being zero, one or two;
R⁵ is hydrogen, C₃₋₇cycloalkyl, aryl, Het¹, Het², C(═O)—R¹⁰, OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³; R⁶ is hydrogen, aryl, C₃₋₇cycloalkyl, Het¹, Het², OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁷ is hydrogen, aryl, C₃₋₇cycloalkyl, Het¹, Het², C(═O)—R¹⁰, S(═O)_{y}—R¹¹, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁸ is hydrogen, aryl, Het¹, Het², C₁₋₆alkyl, C₂₋₆alkenyl, C₂₋₆alkynyl, C₃₋₇cycloalkyl or polyhaloC₁₋₆alkyl;
R⁹ is hydrogen, aryl, C₃₋₇cycloalkyl, Het¹, Het², C(═O)—R¹⁰, S(═O)_{y}—R¹¹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹² and NR⁸R¹³;
R¹⁰ is hydrogen, C₃₋₇cycloalkyl, aryl, Het¹, Het², C(═O)—R⁸, C(═O)—OR⁸, C(═O)—NR⁸R⁸, OR⁸, O—C(═O)—R⁸, O—S(═O)_{y}—R⁸, S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹¹ is hydrogen, C₃₋₇cycloalkyl, aryl, Het¹, Het², OR⁸, O—C(═O)—R⁸, O—S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹² is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹³ is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or, optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹⁴ is hydrogen, phenyl, C₁₋₆alkyl, C₂₋₆alkenyl, C₂₋₆alkynyl, C₃₋₇cycloalkyl;
aryl as a group or part of a group represents a monocyclic or polycyclic aromatic or a partially saturated monocyclic or polycyclic carbocycles wherein any such carbocycle within the meaning of aryl may have up to 14 carbon atoms and may be optionally substituted with one or more substituents independently selected from halogen, nitro, oxo, cyano, C₃₋₇cycloalkyl, Het¹, Het², C(═O)—R⁸, S(═O)_{y}—R¹⁴, OR¹⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het¹ as a group or part of a group represents a saturated or partially unsaturated monocyclic, bicyclic or tricyclic heterocycle having 3 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het² as a group or part of a group represents an aromatic monocyclic, bicyclic or tricyclic heterocycle having 5 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
with the proviso that compounds:
9-(2,4-Dimethoxy-phenylimino)-9H-benzo[f]isoindole-1,3,4-trione,
9-(2,4-Dimethoxy-phenylimino)-2-phenyl-9H-benzo[f]isoindole-1,3,4-trione,
6,7-Dichloro-9-(2,4-dimethoxy-phenylimino)-2-phenyl-9H-benzo[f]isoindole-1,3,4-trione,
4-[6,7-Dichloro-4-(2,4-dimethoxy-phenylimino)-1,3,9-trioxo-1,3,4,9-tetrahydro-benzo[f]isoindol-2-yl]-benzonitrile,
6,7-Dichloro-9-(4-methoxy-2-methyl-phenylimino)-2-phenyl-9H-benzo[f]isoindole-1,3,4-trione,
9-(4-Dimethylamino-phenylimino)-2-phenyl-9H-benzo[f]isoindole-1,3,4-trione,
4-Diethylamino-9-hydroxy-2-phenyl-benzo[f]isoindole-1,3-dione,
4-(But-3-enyl-ethyl-amino)-9-hydroxy-2-phenyl-benzo[f]isoindole-1,3-dione,
4-(Ethyl-pent-4-enyl-amino)-9-hydroxy-2-phenyl-benzo[f]isoindole-1,3-dione,
4,9-dihydroxy-2-methyl-benzo[f]isoindole-1,3-dione,
4,8-dihydroxy-6-methyl-2-oxa-6-aza-s-indacene-5,7-dione,
5,9-dihydroxy-7-methyl-pyrrolo[3,4-g]quinoline-6,8-dione,
4,9-dihydroxy-2-methyl-pyrrolo[3,4-g]isoquinoline-1,3-dione,
4,9-dihydroxy-2,6-dimethyl-benzo[f]isoindole-1,3-dione,
4,9-dihydroxy-6-methoxy-2-methyl-benzo[f]isoindole-1,3-dione,
5-fluoro-4,9-dihydroxy-2-methyl-benzo[f]isoindole-1,3-dione,
6,7-dichloro-4,9-dihydroxy-2-methyl-benzo[f]isoindole-1,3-dione,
6-cyclohexyl-4,8-dihydroxy-1-thia-6-aza-s-indacene-5,7-dione,
4,9-dihydroxy-6-methyl-2-phenyl-benzo[f]isoindole-1,3-dione,
7-cyclohexyl-5,9-dihydroxy-pyrrolo[3,4-g]quinoline-6,8-dione,
2-cyclohexyl-4,9-dihydroxy-6-methoxy-benzo[f]isoindole-1,3-dione,
7-(3,5-dichloro-phenyl)-5,9-dihydroxy-pyrrolo[3,4-g]quinoline-6,8-dione,
6,7-dichloro-2-(3,5-dichloro-phenyl)-4,9-dihydroxy-benzo[f]isoindole-1,3-dione,
4-hydroxy-benzo[f]isoindole-1,3-dione,
4-hydroxy-2-phenyl-benzo[f]isoindole-1,3-dione,
4-hydroxy-2-phenyl-9-phenylamino-benzo[f]isoindole-1,3-dione,
4,9-dihydroxy-2-phenyl-benzo[f]isoindole-1,3-dione,
4-hydroxy-1-methyl-2-phenyl-1,2-dihydro-benzo[f]indazol-3-one,
6,7-dichloro-4,9-dimethoxy-2-methyl-benzo[f]isoindole-1,3-dione, and
6,7-dichloro-2-(3,5-dichloro-phenyl)-4,9-dimethoxy-benzo[f]isoindole-1,3-dione,
are excluded.

4. A compound according to claim 3 having the formula (I), 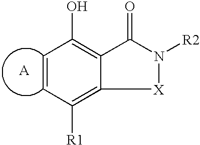
and their N-oxides, salts, stereoisomeric forms, racemic mixtures, prodrugs, esters and metabolites thereof, wherein
X, A, R¹, R², R³, R⁴, R⁵, R⁶, R⁷, R⁸, R⁹, R¹⁰, R¹¹, R¹², R¹³, R¹⁴, y, aryl, Het¹, and Het² are as defined in claim 1, provided that when the A-ring is phenyl, then R² is not hydrogen, methyl, cyclohexyl, nor phenyl;
and compounds
4,8-dihydroxy-6-methyl-2-oxa-6-aza-s-indacene-5,7-dione,
5,9-dihydroxy-7-methyl-pyrrolo[3,4-g]quinoline-6,8-dione,
4,9-dihydroxy-2-methyl-pyrrolo[3,4-g]isoquinoline-1,3-dione,
6-cyclohexyl-4,8-dihydroxy-1-thia-6-aza-s-indacene-5,7-dione,
7-cyclohexyl-5,9-dihydroxy-pyrrolo[3,4-g]quinoline-6,8-dione,
7-(3,5-dichloro-phenyl)-5,9-dihydroxy-pyrrolo[3,4-g]quinoline-6,8-dione,
are excluded.

5. A compound according to claim 3 having the formula (I), 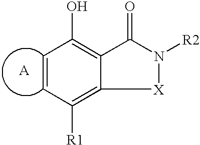
and their N-oxides, salts, stereoisomeric forms, racemic mixtures, prodrugs, esters and metabolites thereof, wherein
X is 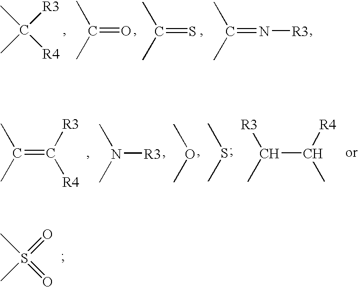
A, also mentioned as “A-ring”, together with the two carbons of the phenyl ring to which it is attached forms a monocyclic Het²;
R¹ is hydrogen, halogen, nitro, cyano, sultam, sultim, C₃₋₇cycloalkyl, C(═O)—R⁵, S(═O)_{y}—R⁶, OR⁷, NR⁸R⁹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R² is hydrogen, C₃₋₅cycloalkyl, C₇cycloalkyl, aryl, Het¹, Het², C(═O)—R⁵, S(═O)_{y}—R⁶, OR⁷, NR⁸R⁹, C(═NR⁸)—R⁵, C₂₋₆alkyl or polysubstituted C₁₋₆alkyl, optionally polysubstituted C₂₋₆alkenyl or optionally polysubstituted C₂₋₆alkynyl; whereby the substituents on C₁₋₆alkyl, and the optional substituents on C₂₋₆alkenyl and C₂₋₆alkynyl are each independently selected from halogen, nitro, cyano, C₃₋₇cycloalkyl, aryl, Het¹, Het², C(═O)—R⁵, S(═O)_{y}—R⁶, OR⁷, and NR⁸R⁹;
R³ is hydrogen, halogen, nitro, cyano, C₃₋₇cycloalkyl, aryl, C(═O)—R⁵, S(═O)_{y}—R⁶, OR⁷, NR⁸R⁹, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, C(═O)—R⁵, OR⁷, and NR⁸R⁹;
R⁴ is hydrogen, halogen, nitro, cyano, C₃₋₇cycloalkyl or C₁₋₆alkyl;
y represents an integer being zero, one or two;
R⁵ is hydrogen, C₃₋₇cycloalkyl, aryl, Het¹, Het², C(═O)—R¹⁰, OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁶ is hydrogen, aryl, C₃₋₇cycloalkyl, Het¹, Het², OR¹², NR⁸R¹³, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁷ is hydrogen, aryl, C₃₋₇cycloalkyl, Het¹, Het², C(═O)—R¹⁰, S(═O)_{y}—R¹¹, or optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹², and NR⁸R¹³;
R⁸ is hydrogen, aryl, Het¹, Het², C₁₋₆alkyl, C₂₋₆alkenyl, C₂₋₆alkynyl, C₃₋₇cycloalkyl or polyhaloC₁₋₆alkyl;
R⁹ is hydrogen, aryl, C₃₋₇cycloalkyl, Het¹, Het², C(═O)—R¹⁰, S(═O)_{y}—R¹¹, C(═NR⁸)—R⁵, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R¹⁰, S(═O)_{y}—R¹¹, OR¹² and NR⁸R¹³;
R¹⁰ is hydrogen, C₃₋₇cycloalkyl, aryl, Het¹, Het², C(═O)—R⁸, C(═O)—OR⁸, C(═O)—NR⁸R⁸, OR⁸, O—C(═O)—R⁸, O—S(═O)_{y}—R⁸, S(═O)_{y}R⁸, NR⁸, R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹¹ is hydrogen, C₃₋₇cycloalkyl, aryl, Het¹, Het², OR⁸, O—C(═O)—R⁸, O—S(═O)_{y}—R⁸, NR⁸R⁸, NR⁸—C(═O)—R⁸, NR⁸—S(═O)_{y}—R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)_{y}—R⁸;
R¹² is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, OR⁸, O—C(═O)—R⁸, O—S(═O)_{y}—R⁸, NR⁸R⁸, NR⁸—C(═O)—R⁸, and NR⁸—S(═O)—R⁸;
R¹³ is hydrogen, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸, optionally polysubstituted C₁₋₆alkyl, optionally polysubstituted C₂₋₆alkenyl or optionally polysubstituted C₂₋₆alkynyl; whereby the optional substituents on C₁₋₆alkyl, C₂₋₆alkenyl and C₂₋₆alkynyl are each independently selected from halogen, nitro, cyano, C₃₋₇cycloalkyl, aryl, Het¹, Het², C(═O)—R⁸, C(═O)—OR⁸, C(═O)—NR⁸R⁸, S(═O)_{y}—R⁸, S(═O)_{y}—OR⁸, S(═O)_{y}—NR⁸R⁸OR⁸, O—C(═O)—R⁸, O—S(═O)_{y}—R⁸, NR⁸R⁸, NR⁸—C(═O)—R⁸, and NR⁸—S(═O)_{y}—R⁸;
R¹⁴ is hydrogen, phenyl, C₁₋₆alkyl, C₂₋₆alkenyl, C₂₋₆alkynyl, C₃₋₇cycloalkyl;
aryl as a group or part of a group represents a monocyclic or polycyclic aromatic or a partially saturated monocyclic or polycyclic carbocycles wherein any such carbocycle within the meaning of aryl may have up to 14 carbon atoms and may be optionally substituted with one or more substituents independently selected from halogen, nitro, oxo, cyano, C₃₋₇cycloalkyl, Het¹, Het², C(═O)—R⁸, S(═O)_{y}—R¹⁴, OR¹⁴, NR¹⁴R¹⁴, NR¹⁴—O—C(═O)—R¹⁴, NR¹⁴—C₁₋₆alkanediyl-NR¹⁴-Het¹, NR¹⁴—C₁₋₆alkanediyl-NR¹⁴-Het², optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het¹ as a group or part of a group represents a saturated or partially unsaturated monocyclic, bicyclic or tricyclic heterocycle having 3 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
Het² as a group or part of a group represents an aromatic monocyclic, bicyclic or tricyclic heterocycle having 5 to 14 ring members, which contains one or more heteroatom ring members selected from nitrogen, oxygen and sulfur, and which may be optionally substituted on a carbon atom or where possible a nitrogen atom with one or more substituents independently selected from halogen, nitro, oxo, cyano, C₃₋₇cycloalkyl, C(═O)—R¹⁴, S(═O)_{y}—R¹⁴, OR¹⁴, NR¹⁴R¹⁴, NR¹⁴—O—C(═O)—R¹⁴, optionally polysubstituted C₁₋₆alkyl, optionally polysubstituted C₂₋₆alkenyl, optionally polysubstituted C₂₋₆alkynyl and optionally polysubstituted phenyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, and NR¹⁴R¹⁴; and whereby the optional substituents on phenyl are each independently selected from halogen, hydroxy, C₁₋₆alkyl, polyhaloC₁₋₆alkyl, O—C₁₋₆alkyl, and C₁₋₆alkanediyl-NR¹⁴R¹⁴;
with the proviso that compound 7-(3,5-dichloro-phenyl)-5,9-dihydroxy-pyrrolo[3,4-g]quinoline-6,8-dione is excluded.

6. A compound according to claim 1, wherein the compound has the formula (IIa) 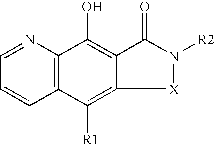
whereby
the pyridinyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴;
and whereby
R² is not 3,5-dichlorophenyl, nor cyclohexyl, nor methyl.

7. A compound according to claim 1, wherein the compound has the formula (IIb) 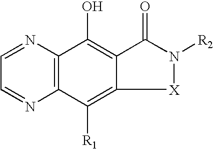
whereby the pyrazinyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

8. A compound according to claim 1, wherein the compound has the formula (IIc) 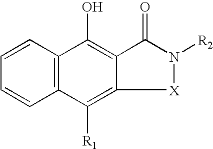
whereby the phenyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴; and whereby R² is not hydrogen, methyl, cyclohexyl, nor phenyl.

9. A compound according to claim 1, wherein the compound has the formula (IId) 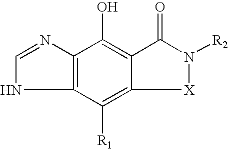
whereby the imidazolyl ring may optionally be substituted with halogen or optionally polysubstituted C₁₋₆alkyl, optionally polysubstituted C₂₋₆alkenyl, optionally polysubstituted C₂₋₆alkynyl; whereby the optional substituents on C₁₋₆alkyl, C₂₋₆alkenyl and C₂₋₆alkynyl are each independently selected from halogen, nitro, cyano, phenyl, C(═O)—R¹⁴, OR¹⁴, Het¹, Het², C(═O)-Het¹, C(═O)-Het², and NR¹⁴R¹⁴.

10. A compound according to claim 1 wherein the compound has the formula (III)

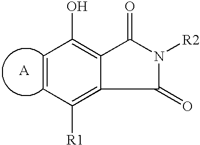

11. A compound according to claim 1, wherein
X is —C(═O)—;
R¹ is —R⁷;
R² is hydrogen, C₃₋₇cycloalkyl, aryl, Het¹, Het², or optionally substituted C₁₋₆alkyl; whereby the optional substituent on C₁₋₆alkyl is selected from C₃₋₇cycloalkyl, aryl, Het¹, Het², and preferably is C₃₋₇cycloalkyl, aryl, Het¹.

12. A compound selected from any of the following compounds:
7-(4-Chloro-benzyl)-5,9-dihydroxy-pyrrolo[3,4-g]quinoxaline-6,8-dione
7-(5-Bromo-2-fluoro-benzyl)-5,9-dihydroxy-pyrrolo[3,4-g]quinoxaline-6,8-dione
7-Benzo[1,3]dioxol-5-ylmethyl-5-(benzyl-methyl-amino)-9-hydroxy-pyrrolo[3,4-g]quinoline-6,8-dione
Dodecanoic acid 7-benzo[1,3]dioxol-5-ylmethyl-9-hydroxy-6,8-dioxo-7,8-dihydro-6H-pyrrolo[3,4-g]quinoxalin-5-yl ester
Acetic acid 9-acetoxy-7-(3,4-dichloro-benzyl)-6,8-dioxo-7,8-dihydro-6H-pyrrolo[3,4-g]quinoxalin-5-yl ester
7-(3,5-Dichloro-benzyl)-5,9-dihydroxy-pyrrolo[3,4-g]quinoxaline-6,8-dione
7-(3,4-Dichloro-benzyl)-5,9-dihydroxy-pyrrolo[3,4-g]quinoxaline-6,8-dione
7-(3-Chloro-benzyl)-5,9-dihydroxy-pyrrolo[3,4-g]quinoxaline-6,8-dione
Dicyclopropanecarboxylic acid 7-(3,4-dichloro-benzyl)-6,8-dioxo-7,8-dihydro-6H-pyrrolo[3,4-g]quinoxalin-5,9-diyl ester
7-(3-Bromo-4-fluoro-benzyl)-5,9-dihydroxy-pyrrolo[3,4-g]quinoxaline-6,8-dione
7-(3-Bromo-benzyl)-5,9-dihydroxy-2-methyl-pyrrolo[3,4-g]quinoxaline-6,8-dione
7-Benzo[1,3]dioxol-5-ylmethyl-5,9-dihydroxy-2-methyl-pyrrolo[3,4-g]quinoxaline-6,8-dione
7-(3,4-Dichloro-benzyl)-5,9-dihydroxy-2-methyl-pyrrolo[3,4-g]quinoxaline-6,8-dione
7-(3-Bromo-benzyl)-5,9-dihydroxy-pyrrolo[3,4-g]quinoxaline-6,8-dione

13. A pharmaceutical composition, comprising an effective amount of at least one compound selected from any of the following compounds:
7-(4-Chloro-benzyl)-5,9-dihydroxy-pyrrolo[3,4-g]quinoxaline-6,8-dione
7-(5-Bromo-2-fluoro-benzyl)-5,9-dihydroxy-pyrrolo[3,4-g]quinoxaline-6,8-dione
7-Benzo[1,3]dioxol-5-ylmethyl-5-(benzyl-methyl-amino)-9-hydroxy-pyrrolo[3,4-g]quinoline-6,8-dione
Dodecanoic acid 7-benzo[1,3]dioxol-5-ylmethyl-9-hydroxy-6,8-dioxo-7,8-dihydro-6H-pyrrolo[3,4-g]quinoxalin-5-yl ester
Acetic acid 9-acetoxy-7-(3,4-dichloro-benzyl)-6,8-dioxo-7,8-dihydro-6H-pyrrolo[3,4-g]quinoxalin-5-yl ester
7-(3,5-Dichloro-benzyl)-5,9-dihydroxy-pyrrolo[3,4-g]quinoxaline-6,8-dione
7-(3,4-Dichloro-benzyl)-5,9-dihydroxy-pyrrolo[3,4-g]quinoxaline-6,8-dione
7-(3-Chloro-benzyl)-5,9-dihydroxy-pyrrolo[3,4-g]quinoxaline-6,8-dione
Dicyclopropanecarboxylic acid 7-(3,4-dichloro-benzyl)-6,8-dioxo-7,8-dihydro-6H-pyrrolo[3,4-g]quinoxalin-5,9-diyl ester
7-(3-Bromo-4-fluoro-benzyl)-5,9-dihydroxy-pyrrolo[3,4-g]quinoxaline-6,8-dione
7-(3-Bromo-benzyl)-5,9-dihydroxy-2-methyl-pyrrolo[3,4-g]quinoxaline-6,8-dione
7-Benzo[1,3]dioxol-5-ylmethyl-5,9-dihydroxy-2-methyl-pyrrolo[3,4-g]quinoxaline-6,8-dione
7-(3,4-Dichloro-benzyl)-5,9-dihydroxy-2-methyl-pyrrolo[3,4-g]quinoxaline-6,8-dione
7-(3-Bromo-benzyl)-5,9-dihydroxy-pyrrolo[3,4-g]quinoxaline-6,8-dione
and a pharmaceutically acceptable excipient.
